(19) United States
(12) Patent Application Publication
(10) Pub. No.: US 2025/0066394 A1
(43) Pub. Date: Feb. 27, 2025

(54) 3-(2-(DIMETHYLAMINO)ETHYL)-1H-INDOL-4-yl DERIVATIVES

(71) Applicant: Compass Pathfinder Limited, Altrincham (GB)

(72) Inventors: John Richard MORPHY, Altrincham (GB); Thomas Matthew BAKER, Nottingham (GB); Andrew William BROWN, Nottingham (GB)

(21) Appl. No.: 18/813,131

(22) Filed: Aug. 23, 2024

Related U.S. Application Data
(60) Provisional application No. 63/578,791, filed on Aug. 25, 2023.

(51) Int. Cl. C07F 7/08; A61K 31/4045; A61K 31/4155; A61K 31/454; A61K 31/675; A61K 31/695; C07D 209/16; C07D 401/12; C07D 403/12; C07F 9/572

(52) U.S. Cl. CPC C07F 7/0812; A61K 31/4045; A61K 31/4155; A61K 31/454; A61K 31/675; A61K 31/695; C07D 209/16; C07D 401/12; C07D 403/12; C07F 9/5728

(57) ABSTRACT

Provided herein are compounds of Formula (I),

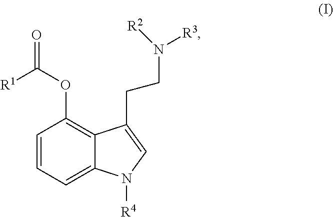

a compound selected from any of the compounds in Table 1, Table 2, Table 3, or a pharmaceutically acceptable salt or deuterated form thereof, wherein R¹, R², R³ and R⁴ are defined herein. Also provided herein are pharmaceutical compositions comprising a compound of formula (I), or a compound selected from any of the compounds in Table 1, Table 2, Table 3, or pharmaceutically acceptable salt or deuterated form thereof, and methods of using a compound of formula (I) or pharmaceutically acceptable salt or deuterated form thereof, e.g., in treating 5-HT₂A receptor associated diseases or disorders.

### CROSS-REFERENCE TO RELATED APPLICATIONS

This application claims priority to U.S. Provisional Application No. 63/578,791, filed on Aug. 25, 2023, the disclosure of which is incorporated by reference in its entirety for all purposes.

### BACKGROUND

Over 50% of adults in the United States will be diagnosed with a psychiatric disorder at some point in their lifetime. Nearly 1 in 5 suffer from mental illness, and nearly 1 in 25 are afflicted with severe mental illness, such as major depression, schizophrenia, or bipolar disorder.

Psychedelics show promising activity in treating mental illness. New psychedelic compounds are needed for treating mental illness.

### BRIEF DESCRIPTION

In some embodiments, the present disclosure provides a compound of formula (I):

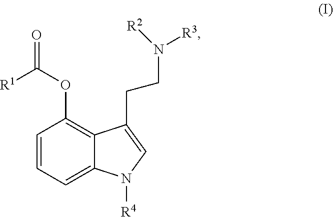

or pharmaceutically acceptable salt or deuterated form thereof,
   wherein:
R² and R³ are independently alkyl;
R⁴ is H or C(═O)Oalkyl; and
R¹ is defined according to i, ii, iii, or iv:
   i. R¹ is —OR⁸, wherein R⁸ is alkynyl substituted with NR⁵R⁶, or —(CH₂CH₂O)ₘ-alkyl, wherein m is 1, 2, 3, 5 or 6;
      R⁵ and R⁶ are independently H, alkyl, C(═O)alkyl, alkylene-aryl, or S(O)₂alkyl;
   ii. R¹ is —R^{9A}, wherein R^{9A} is —C(R^{A})₂NR⁵R⁶, —C(R^{A})₂—O-alkyl, —C(R^{A})₂—O-cycloalkyl, cycloalkyl substituted with Oalkyl, heterocyclyl substituted with C(═O)Oalkyl, alkenylene-aryl wherein the aryl is substituted with 1-4 R^{7A}, alkenylene-heteroaryl wherein the heteroaryl is substituted with 0-4 R^{7A}, or aryl substituted with NR⁵R⁶;
      each R^{A} is independently H, halo, unsubstituted alky, or alkylene-OH, or
      two R^{A} together with the carbon they are connected to form a cycloalkylene or heterocyclylene,
      provided that at least one R^{A} is not H;
      R⁵ and R⁶ are independently H, alkyl, or

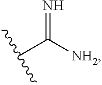 [SMILES: *C(=N)N]

or C(═O)alkyl wherein the alkyl is optionally substituted with NH₂, NH(alkyl), N(alkyl)₂, C(═O)aryl, C(═O)Oalkyl, S(O)₂alkyl, or alkylene-heterocyclyl;
R^{7A} is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl;
provided that R^{9A} is not

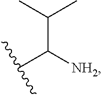 [SMILES: *C(N)C(C)C]

iii. R¹ is —(CH₂)ₙ—R^{9B}, wherein
   n is 1, 2, 3, 4, 5, or 6;
   R^{9B} is —O-alkylene-OH, —C(═O)-alkylene-NR⁵R⁶, alkylene-aryl wherein the aryl is substituted with 1-4 R^{7B}, Si(alkyl)₃ or —(OCH₂CH₂)ₘ—Oalkyl, m is 2 or 3, 5 or 6, provided that when R^{9B} is Si(alkyl)₃ or

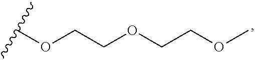 [SMILES: *OCCOCCOC]

then n is not 1;
R⁵ and R⁶ are independently H, alkyl, C(═O)alkyl, C(═O)aryl, C(═O)Oalkyl, S(O)₂alkyl, alkylene-heterocyclyl;
R^{7B} is —OP(═O)OH₂, C₁₋₆ alkyl;
iv. R¹ is —N(R^{5A})(R^{6A}), wherein
   R^{5A} and R^{6A} are independently H, C₁₋₆ alkyl optionally substituted with —COOH, —OC(═O)alkyl or —O-alkylene-OH,
   provided that when one of R^{5A} and R^{6A} is H, the other cannot be H, CH₂CH(CH₃)₂ or (CH₂)₅CH₃, and
   further provided that when one of R^{5A} and R^{6A} is CH₃, the other cannot be H, CH₃ or CH₂CH₃, CH₂CH(CH₃)₂,

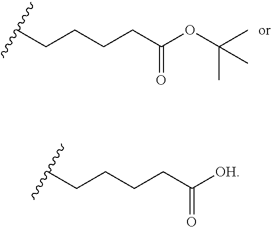 [SMILES: *CCCCC(=O)OC(C)(C)C ; *CCCCC(=O)O]

In some embodiments, this disclosure provides a compound of formula (I),

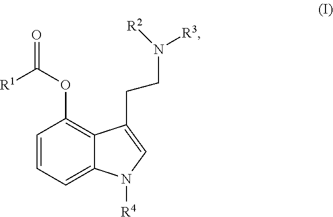

or pharmaceutically acceptable salt or deuterated form thereof,
   wherein:
R² and R³ are independently alkyl;
R⁴ is H or C(═O)Oalkyl; and
R¹ is defined according to i, ii or iii:
i. R¹ is —OR⁸, wherein R⁸ is —(CH₂CH₂O)ₘ-alkyl, m is 2, 3, 5 or 6;
ii. R¹ is —R^{9A}, wherein R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, 5-membered heteroaryl comprising two N atoms wherein the heteroaryl is substituted with C₁₋₃ alkyl, or arylene-NR⁵R⁶;
R⁵ and R⁶ are independently H, alkyl,

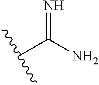 [SMILES: *C(=N)N]

or C(═O)alkyl wherein the alkyl is optionally substituted with NH₂, NH(alkyl) or N(alkyl)₂,
each R^{A} is independently H, halo, unsubstituted alky, or alkylene-OH, or
two R^{A} together with the carbon they are connected to form a cycloalkylene or heterocyclylene,
provided that at least one R^{A} is not H;
iii. R¹ is —(CH₂)ₙ—R^{9B}, wherein
   n is 1, 2, 3, 4, 5, or 6;
   R^{9B} is − branched alkylene-aryl wherein the aryl is substituted with 1-4 R^{7B}, Si(alkyl)₃ or —(OCH₂CH₂)ₘ—Oalkyl;
   R^{7B} is —OP(═O)OH₂, C₁₋₆ alkyl,
   provided that when R^{9B} is Si(alkyl)₃ or

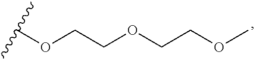 [SMILES: *OCCOCCOC]

then n is not 1.

### DETAILED DESCRIPTION

### Definitions

The terms below, as used herein, have the following meanings, unless indicated otherwise:

“Cyano” refers to the —CN radical.

“Hydroxy” or “hydroxyl” refers to the —OH radical.

“Oxo” refers to the ═O substituent.

“Alkyl” or “alkyl group” refers to a fully saturated, straight, or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl”, “arylalkyl” or “alkyene-aryl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a non-aromatic ring structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, e.g., having from three to ten carbon atoms, and which is attached to the rest of the molecule by a single bond.

Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkene” refers to a divalentnon-aromatic monocyclic or polycyclic fully saturated hydrocarbon ring consisting solely of carbon and hydrogen atoms, which can include fused, spiro, or bridged ring systems, having from three to twenty carbon atoms, e.g., having from three to ten carbon atoms, and which is attached to the rest of the molecule by one or more single bonds. Monocyclic cycloalkylene include, for example, cyclopropylene, cyclobutylene, cyclopentylene, cyclohexylene, cycloheptylene, and cyclooctylene. Polycyclic cycloalkylene includes, for example, bicyclo[2.2.2]octanylene, cubanylene, bicyclo(1.1.1)pentylene, adamantylene, norbornylene, decalinylene, 7,7-dimethyl-bicyclo[2.2.1]heptanylene, and the like. Unless otherwise stated specifically in the specification, a cycloalkylene group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, e.g., having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, e.g., having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like.

Unless stated otherwise specifically in the specification, a haloalkynyl group can be optionally substituted.

“Heterocyclyl” “heterocyclic ring” or “heterocycle” refers to a stable 3- to 20-membered non-aromatic radical which consists of two to twelve carbon atoms and from one to six heteroatoms selected from nitrogen, oxygen, sulfur, or silicon. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated.

Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylene” refers to a multivalent (e.g., divalent, trivalent or tetravalent) 3- to 20-membered non-aromatic, partially aromatic, or aromatic ring radical which consists of two to twelve carbon atoms and from one to six heteroatoms selected from nitrogen, oxygen or sulfur. Unless stated otherwise specifically in the specification, the heterocyclylene radical can be a monocyclic, bicyclic, tricyclic or tetracyclic ring system, which can include fused, spiro, or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclylene radicals include, but are not limited to, dioxolanylene, decahydroisoquinolylene, imidazolinylene, imidazolidinylene, isothiazolidinylene, isoxazolidinylene, morpholinylene, octahydroindolylene, octahydroisoindolylene, 2-oxopiperazinylene, 2-oxopiperidinylene, 2-oxopyrrolidinylene, oxazolidinylene, piperidinylene, piperazinylene, 4-piperidonylene, pyrrolidinylene, pyrazolidinylene, quinuclidinylene, thiazolidinylene, tetrahydrofurylene, trithianylene, tetrahydropyranylene, thiomorpholinylene, thiamorpholinylene, 1-oxo-thiomorpholinylene, and 1,1-dioxo-thiomorpholinylene. Unless stated otherwise specifically in the specification, a heterocyclylene group can be optionally substituted.

“Heterocyclylalkyl” or “alkylene-heterocyclyl” refers to a radical of the formula —R_{b}—Rₑ where R_{b} is an alkylene group as defined above and Rₑ is a heterocyclyl radical as defined above. Unless stated otherwise specifically in the specification, a heterocycloalkyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring comprising at least one heteroatom selected from nitrogen, oxygen and sulfur. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” or “alkylene-heteroaryl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl.

“Substituted” further includes any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

In this specification, unless stated otherwise, the term “pharmaceutically acceptable” is used to characterize a moiety (e.g., a salt, dosage form, or excipient) as being appropriate for therapeutic use. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

In some embodiments, “substituted” further means any alkyl, cycloalkyl or heterocyclylalkyl in which one or more hydrogen atoms is replaced by an isotope e.g., deuterium. In addition, each of the foregoing substituents can also be optionally substituted with one or more of the above substituents.

The compounds of the present disclosure may also contain unnatural proportions of atomic isotopes at one or more of the atoms that constitute such compounds. For example, the compounds may be radiolabeled with radioactive isotopes, such as for example deuterium (²H), tritium (³H), iodine-125 (¹²⁵I) or carbon-14 (¹⁴C). All isotopic variations of the compounds of the present disclosure, whether radioactive or not, are intended to be encompassed within the scope of the disclosure.

In some examples, unless otherwise specified, “rt” or “r.t.” means room temperature, “h” or “h.” means hour, “min” or “min.” means minutes, and “eq” means equivalent.

### COMPOUNDS

Disclosed herein are compounds that release psilocin when administered to a subject in need thereof. The disclosed compounds are designed to release psilocin after administration to a subject in need thereof. In embodiments, the disclosed compounds have a psilocin release efficacy (as described in Example 10) ranging from about 7% to about 100%, including about 25% to about 100%, about 50% to about 100%, about 75% to about 100%, about 80% to about 100%, about 85% to about 100%, about 90% to about 100% or about 95% to about 100%. In embodiments, the disclosed compounds have a release efficacy of about 80% to about 100%.

In some embodiments, this disclosure provides a compound of formula (I),

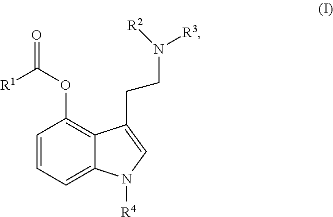

or pharmaceutically acceptable salt or deuterated form thereof,
   wherein:
R² and R³ are independently alkyl;
R⁴ is H or C(═O)Oalkyl; and
R¹ is defined according to i, ii, iii, or iv:
   i. R¹ is —OR⁸, wherein R⁸ is alkynyl substituted with NR⁵R⁶, or —(CH₂CH₂O)ₘ alkyl, wherein m is 1, 2, 3, 5 or 6;
      R⁵ and R⁶ are independently H, alkyl, C(═O)alkyl, alkylene-aryl, or S(O)₂alkyl;
   ii. R¹ is —R^{9A}, wherein R^{9A} is —C(R^{A})₂NR⁵R⁶, —C(R^{A})₂—O-alkyl, —C(R^{A})₂—O-cycloalkyl, cycloalkyl substituted with Oalkyl, heterocyclyl substituted with C(═O)Oalkyl, alkenylene-aryl wherein the aryl is substituted with 1-4 R^{7A}, alkenylene-heteroaryl wherein the heteroaryl is substituted with 0-4 R^{7A}, or aryl substituted with NR⁵R⁶, or 5-membered heteroaryl substituted with 1-4 groups selected from alkyl, OH, halogen, haloalkyl, Oalkyl or O-haloalkyl;
      each R^{A} is independently H, halo, unsubstituted alkyl, or alkylene-OH, or
      two R^{A} together with the carbon they are connected to form a cycloalkylene or heterocyclylene,
      provided that at least one R^{A} is not H;
      R⁵ and R⁶ are independently H, alkyl, or

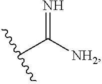 [SMILES: *C(=N)N]

or C(═O)alkyl wherein the alkyl is optionally substituted with NH₂, NH(alkyl), N(alkyl)₂, C(═O)aryl, C(═O)Oalkyl, S(O)₂alkyl, or alkylene-heterocyclyl;
R^{7A} is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl;
provided that R^{9A} is not

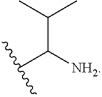 [SMILES: *C(N)C(C)C]

iii. R¹ is —(CH₂)ₙ—R^{9B}, wherein
   n is 1, 2, 3, 4, 5, or 6;
   R^{9B} is —O-alkylene-OH, —C(═O)-alkylene-NR⁵R⁶, alkylene-aryl wherein the aryl is substituted with 1-4 R^{7B}, Si(alkyl)₃ or —(OCH₂CH₂)ₘ—Oalkyl, m is 2 or 3,
   provided that when R^{9B} is Si(alkyl)₃ or

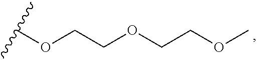 [SMILES: *OCCOCCOC]

then n is not 1;
R⁵ and R⁶ are independently H, alkyl, C(═O)alkyl, C(═O)aryl, C(═O)Oalkyl, S(O)₂alkyl, alkylene-heterocyclyl;
R^{7B} is —OP(═O)OH₂, C₁₋₆ alkyl;
iv. R¹ is —N(R^{5A})(R^{6A}), wherein
   R^{5A} and R^{6A} are independently H, C₁₋₆ alkyl optionally substituted with —COOH, —OC(═O)alkyl or —O-alkylene-OH,
   provided that when one of R^{5A} and R^{6A} is H, the other cannot be H, CH₂CH(CH₃)₂ or (CH₂)₅CH₃, and
   further provided that when one of R^{5A} and R^{6A} is CH₃, the other cannot be H, CH₃, CH₂CH₃, CH₂CH(CH₃)₂ or

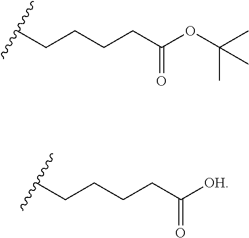 [SMILES: *CCCCC(=O)OC(C)(C)C ; *CCCCC(=O)O]

In some embodiments of formula (I), R^{9A} is not

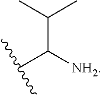 [SMILES: *C(N)C(C)C]

In some embodiments of formula (I), when one of R^{5A} and R^{6A} is H, the other cannot be H, CH₂CH(CH₃)₂ or (CH₂)₅CH₃.

In some embodiments of formula (I), when one of R^{5A} and R^{6A} is CH₃, the other cannot be H, CH₃, CH₂CH₃, CH₂CH(CH₃)₂,

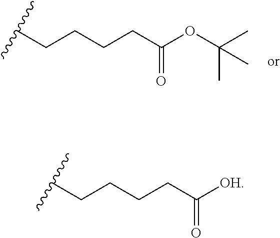 [SMILES: *CCCCC(=O)OC(C)(C)C ; *CCCCC(=O)O]

In some embodiments, the compound is a compound of Formula (II)

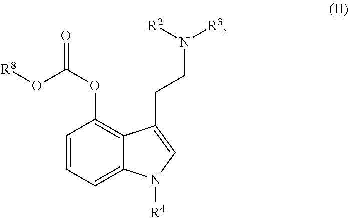

or a pharmaceutically acceptable salt or deuterated form thereof,
wherein:
R⁸ is alkynylene-NR⁵R⁶ or —(CH₂CH₂O)ₘ-alkyl;
m is 1, 2, 3, 5 or 6;
R² and R³ are independently alkyl;
R⁴ is H or C(═O)Oalkyl; and
R⁵ and R⁶ are independently H, alkyl, C(═O)H, C(═O)alkyl, alkylene-heteroaryl, alkylene-aryl, alkylene-carbocyclyl, alkylene-heterocyclyl, S(═O)alkyl, or S(═O)₂alkyl.

In some embodiments of formula (II), R⁸ is: C₁₋₆ alkynyl substituted with N(C₁₋₆ alkyl)₂, or —(CH₂CH₂O)ₘ—CH₃, wherein m is 1, 2 or 3. In some embodiments, R⁸ is C₁₋₆ alkynyl substituted with N(C₁₋₆ alkyl)₂. In some embodiments, R⁸ is —(CH₂CH₂O)ₘ—CH₃. In some embodiments, m is 1. In some embodiments, m is 2. In some embodiments m is 3.

In some embodiments, R⁸ is

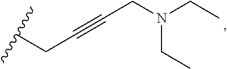 [SMILES: *CC#CCN(CC)CC]

—(CH₂CH₂O)—CH₃, —(CH₂CH₂O)₂—CH₃ or —(CH₂CH₂O)₃—CH₃.

In some embodiments, compound is a compound of Formula (III)

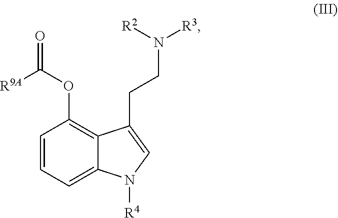

or a pharmaceutically acceptable salt or deuterated form thereof,
wherein:
R^{9A} is —C(R^{A})₂NR⁵R⁶, —C(R^{A})₂—O-alkyl, —C(R^{A})₂—O-cycloalkyl, cycloalkyl substituted with Oalkyl, heterocyclyl substituted with C(═O)Oalkyl, alkenylene-aryl wherein the aryl is substituted with 1-4 R^{7A}, alkenylene-heteroaryl wherein the heteroaryl is substituted with 0-4 R^{7A}, aryl substituted with NR⁵R⁶ or 5-membered heteroaryl substituted with 1-4 groups selected from alkyl, OH, halogen, haloalkyl, Oalkyl or O-haloalkyl;
each R^{A} is independently H, halo, unsubstituted alkyl, or alkylene-OH, or
two R^{A} together with the carbon they are connected to form a cycloalkylene or heterocyclyl,
provided that at least one R^{A} is not H;
R² and R³ are independently alkyl;
R⁴ is H or C(═O)Oalkyl;
R⁵ and R⁶ are independently H, alkyl,

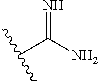 [SMILES: *C(=N)N]

or C(═O)alkyl wherein the alkyl is optionally substituted with NH₂, NH(alkyl) or N(alkyl)₂;
R^{7A} is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl,
   provided that R^{9A} is not

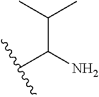 [SMILES: *C(N)C(C)C]

In some embodiments of formula (III), R^{9A} is not

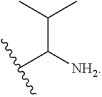 [SMILES: *C(N)C(C)C]

In some embodiments of Formula (III), R^{9A} is selected from —C(R^{A})₂NR⁵R⁶, —C(R^{A})₂—O-C₁₋₆ alkyl, —C(R^{A})₂—O-C₃₋₆cycloalkyl, cyclopropyl substituted with OC₁₋₆ alkyl, heterocyclyl substituted with C(═O)OC₁₋₆alkyl, C₁₋₆ alkenylene-aryl wherein the aryl is substituted with 1-4 R^{7A}, C₁-6 alkenylene-heteroaryl, arylene-NR⁵R⁶, or 5-membered heteroaryl comprising two N atoms wherein the heteroaryl is substituted with C₁₋₃ alkyl.

In some embodiments, R^{9A} is —C(R^{A})₂NR⁵R⁶, —C(R^{A})₂—O-C₁₋₆ alkyl, or —C(R^{A})₂—O-C₃₋₆cycloalkyl. In some embodiments, R^{9A} is —C(R^{A})₂NR⁵R⁶. In some embodiments, R^{9A} is —C(R^{A})₂—O-C₁₋₆ alkyl. In some embodiments, R^{9A} is —C(R^{A})₂—O-C₃₋₆cycloalkyl. In some embodiments, R^{9A} is cyclopropyl substituted with OC₁₋₆ alkyl. In some embodiments, R^{9A} is heterocyclyl substituted with C(═O)OC₁₋₆alkyl. In some embodiments, R^{9A} is 3-8 membered heterocyclyl substituted with C(═O)OC₁₋₆alkyl. In some embodiments, R^{9A} is C₁₋₆ alkenylene-aryl wherein the aryl is substituted with 1-4 R^{7A}. In some embodiments, R^{9A} is C₁₋₆ alkenylene-heteroaryl. In some embodiments, R^{9A} is arylene-NR⁵R⁶. In some embodiments, R^{9A} is 5-membered heteroaryl comprising two N atoms wherein the heteroaryl is substituted with C₁₋₃ alkyl.

In some embodiments, R^{9A} is —C(R^{A})₂NR⁵R⁶. In some embodiments, R⁵ and R⁶ are independently H, alkyl,

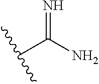 [SMILES: *C(=N)N]

or C(═O)alkyl wherein the alkyl is optionally substituted with NH₂, NH(alkyl) or N(alkyl)₂.

In some embodiments, R^{7A} is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl. In some embodiments, R^{7A} is OH, halo, C₁₋₆ alkyl, haloC₁₋₆alkyl, O-C₁₋₆alkyl, OC(═O) C₁₋₆alky, Si(C₁₋₆alkyl)₃, aryl, NH(C═O)C₁₋₆alkyl, NH₂, NH(C₁₋₆alkyl), N(C₁₋₆alkyl)₂, or 3-8 membered heterocyclyl. In some embodiments, R^{7A} is alkyl. In some embodiments, R^{7A} is C₁₋₆ alkyl. In some embodiments, R^{7A} is C₁₋₃ alkyl. In some embodiments, R^{7A} is CH₃. In some embodiments, each R^{A} is independently H or unsubstituted alkyl, provided that at least one R^{A} is not H.

In some embodiments, R^{9A} is 5-membered heteroaryl substituted with 1-4 groups selected from alkyl, OH, halogen, haloalkyl, Oalkyl or O-haloalkyl, wherein the 5-membered heteroaryl is imidazole, pyrazole, isothiazole, thiazole, isoxazole, triazole or oxazole.

In some embodiments, R^{9A} is 5-membered heteroaryl comprising two N atoms wherein the heteroaryl is substituted with C₁₋₃ alkyl, wherein the 5-membered heteroaryl is imidazole or pyrazole. In some embodiments, R^{9A} is pyrazole substituted with C₁₋₃ alkyl.

In some embodiments, R^{9A} is

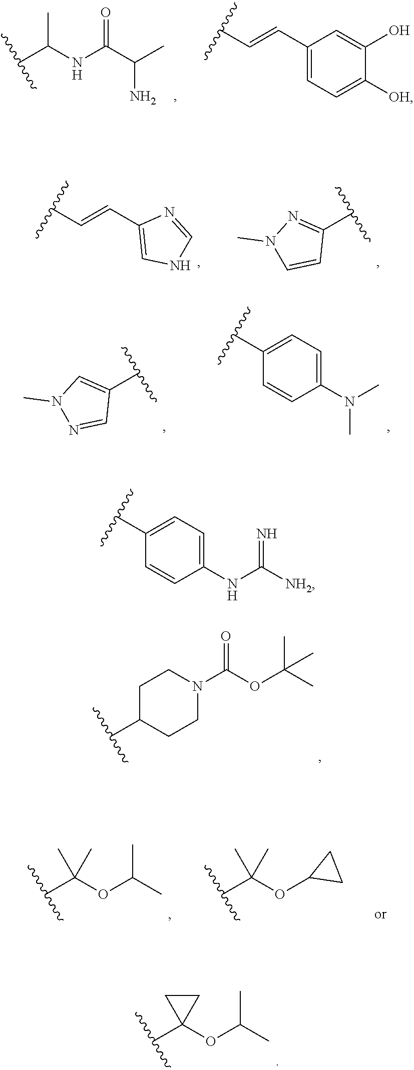

In some embodiments, the compound is a compound of Formula (IV)

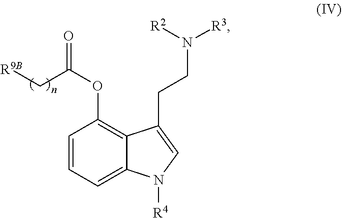

(IV), or a pharmaceutically acceptable salt or deuterated form thereof,
wherein:
n is 1, 2, 3, 4, 5, or 6;
R^{9B} is —O-alkylene-OH, —C(═O)-alkylene-NR⁵R⁶, alkylene-aryl wherein the aryl is
substituted with 1-4 R^{7B}, Si(alkyl)₃ or —(OCH₂CH₂)ₘ—Oalkyl;
m is 2 or 3;
R² and R³ are independently alkyl;
R⁴ is H or C(═O)Oalkyl;
R⁵ and R⁶ are independently H, alkyl, C(═O)alkyl, C(═O)aryl, C(═O)Oalkyl,
S(O)₂alkyl or alkylene-heterocyclyl;
R^{7B} is —OP(═O)OH₂, C₁₋₆ alkyl,
provided that when R^{9B} is Si(alkyl)₃ or

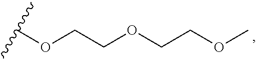 [SMILES: *OCCOCCOC]

then n is not 1.

In some embodiments of formula (IV), when R^{9B} is Si(alkyl)₃ or

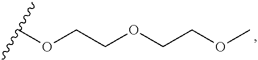 [SMILES: *OCCOCCOC]

then n is not 1.

In some embodiments of formula (IV), R^{9B} is —O-C₁₋₆ alkylene-OH, Si(C₁₋₆alkyl)₃ or

In some embodiments of formula (IV),
n is 1, 2 or 3;
R^{9B} is —O-C₁₋₆ alkylene-OH or —C(═O)—C₁₋₆ alkylene-NR⁵R⁶, C₁₋₆ alkylene-aryl wherein
the aryl is substituted with 1-4 R^{7B}, Si(C₁₋₆ alkyl)₃, —(OCH₂CH₂)ₘ—Oalkyl;
m is 2 or 3;
R² and R³ are independently alkyl;
R⁴ is H or C(═O)OC₁₋₆ alkyl;
R⁵ and R⁶ are independently H or alkyl.

In some embodiments of formula (IV), R^{9B} is —O-alkylene-OH. In some embodiments, R^{9B} is —O-C₁₋₆ alkylene-OH. In some embodiments, R^{9B} is —C(═O)-alkylene-NR⁵R⁶. In some embodiments, R^{9B} is —C(═O)—C₁₋₆ alkylene-NR⁵R⁶. In some embodiments, R^{9B} is alkylene-aryl wherein the aryl is substituted with 1-4 R^{7B}. In some embodiments, R^{9B} is C₁₋₆alkylene-aryl wherein the aryl is substituted with 1-4 R^{7B}. In some embodiments, R^{9B} is —O-alkylene-OH. In some embodiments, R^{9B} is —O-C₁₋₆ alkylene-OH. In some embodiments, R^{9B} is Si(alkyl)₃. In some embodiments, R^{9B} is Si(C₁₋₆alkyl)₃. In some embodiments, R^{9B} is —(OCH₂CH₂)ₘ—Oalkyl. In some embodiments, R^{9B} is —(OCH₂CH₂)ₘ—OC₁₋₆alkyl. In some embodiments, m is 2. In some embodiments, m is 3. In some embodiments, R^{7B} is —OP(═O)OH₂ or C₁₋₃ alkyl. In some embodiments, R^{7B} is —OP(═O)OH₂ or CH₃.

In some embodiments of formula (IV), n is 1. In some embodiments, n is 2. In some embodiments, n is 3. In some embodiments, n is 4. In some embodiments, n is 5. In some embodiments, n is 6.

In some embodiments of formula (IV), R^{9B} is

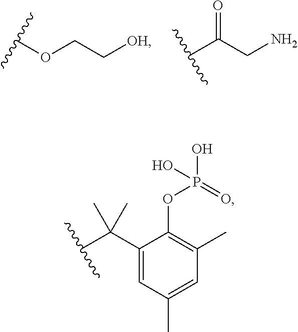 [SMILES: *OCCO ; *C(=O)CN ; *C(C)(C)c1cc(C)cc(C)c1OP(=O)(O)O]

Si(CH₃)₃,

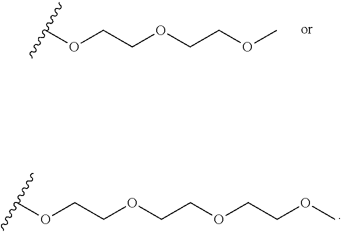

In some embodiments of formula (IV), n is 1, 2 or 3, and R^{9B} is

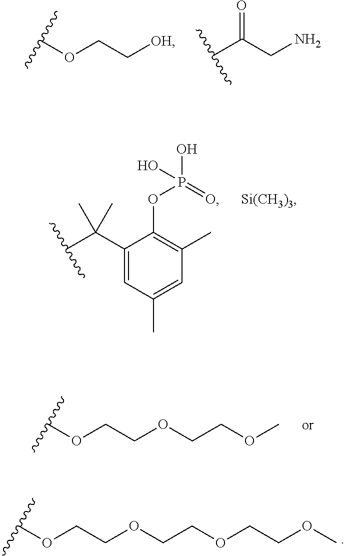

In some embodiments, compound of this disclosure is a compound of Formula (V)

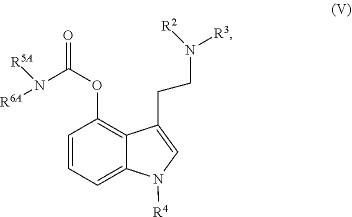

or a pharmaceutically acceptable salt or deuterated form thereof,
wherein:
R² and R³ are independently alkyl;
R⁴ is H or C(═O)Oalkyl;
R^{5A} and R^{6A} are independently H, C₁₋₆ alkyl optionally substituted with —COOH, —OC(═O)alkyl or —O-alkylene-OH,
provided that both R^{5A} and R^{6A} are not H, and
when one of R^{5A} and R^{6A} is H, the other cannot be H, CH₂CH(CH₃)₂, or (CH₂)₅CH₃, and
further provided that when one of R^{5A} and R^{6A} is CH₃, the other cannot be H, CH₃, CH₂CH₃, CH₂CH(CH₃)₂,

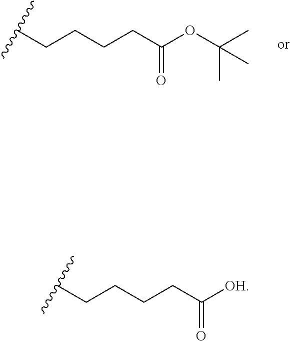 [SMILES: *CCCCC(=O)OC(C)(C)C ; *CCCCC(=O)O]

In some embodiments of formula (V), both R^{5A} and R^{6A} are not H.

In some embodiments of formula (V), when one of R^{5A} and R^{6A} is H, the other cannot be H, CH₂CH(CH₃)₂, or (CH₂)₅CH₃.

In some embodiments of formula (V), when one of R^{5A} and R^{6A} is CH₃, the other cannot be H, CH₃, CH₂CH₃, CH₂CH(CH₃)₂,

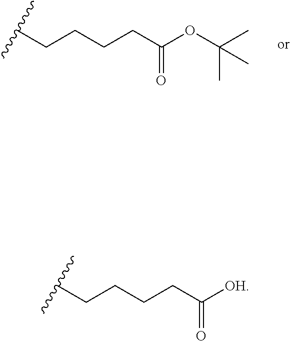 [SMILES: *CCCCC(=O)OC(C)(C)C ; *CCCCC(=O)O]

In some embodiments of formula (V), R^{5A} and R^{6A} are independently H, C₁₋₆ alkyl optionally substituted with —COOH, —OC(═O)C₁₋₆alkyl or —O-C₁₋₆alkylene-OH. In some embodiments of formula (V), R^{5A} is H and R^{6A} is C₁₋₆ alkyl optionally substituted with —COOH, —OC(═O)C₁₋₆alkyl or —O-C₁₋₆alkylene-OH. In some embodiments, R^{5A} is CH₃ and R^{6A} is C₁₋₆ alkyl optionally substituted with —COOH, —OC(═O)C₁₋₆alkyl or —O-C₁₋₆alkylene-OH.

In some embodiments of formula (V), R^{5A} and R^{6A} are independently H, CH₃,

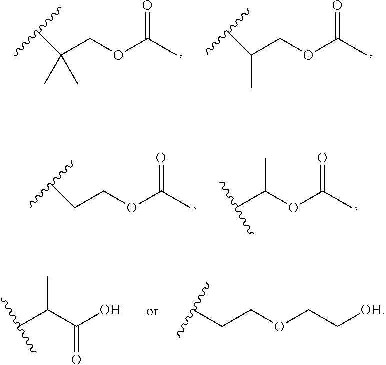

In some embodiments, R^{5A} is H, and R^{6A} is CH₃,

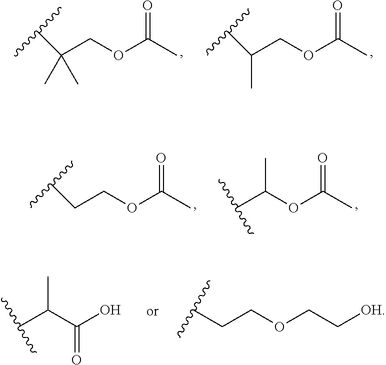

In some embodiments, R^{5A} is H, and R^{6A} is

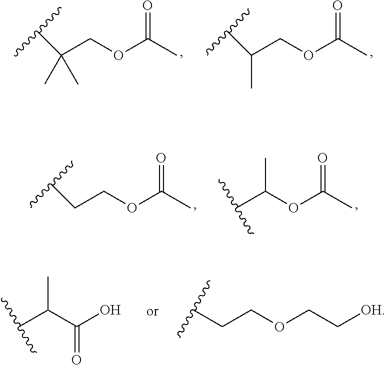

In some embodiments, this disclosure provides a compound of Formula (VI)

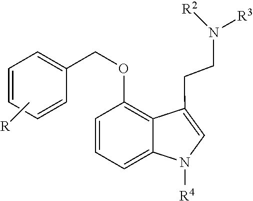

(V), or a pharmaceutically acceptable salt or deuterated thereof,
wherein:
R is NR⁵R⁶;
R² and R³ are independently alkyl;
R⁴ is H or C(═O)Oalkyl;
R⁵ and R⁶ are independently H, alkyl, haloalkyl, or C(═O)haloalkyl.

In some embodiments of formula (VI), R is NR⁵R⁶, and R⁵ and R⁶ are independently H or haloalkyl. In some embodiments, R is NHR⁶, wherein R⁶ is haloalkyl. In some embodiments, R is NHR⁶, wherein R⁶ is haloC₁₋₆alkyl.

In some embodiments, this disclosure provides a compound of Formula (VII)

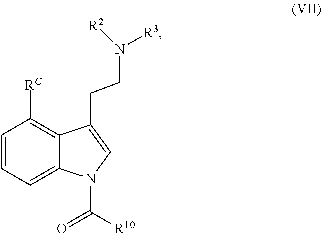

or a pharmaceutically acceptable salt or deuterated form thereof,
wherein:
R² and R³ are independently alkyl;
R^{c} is OH or OC(═O)alkyl;
R¹⁰ is NR^{5A}R^{6A}, O-alkylene-OC(═O)alkyl, O-alkylene-Si(alkyl)₂, heterocycle, or aryl;
R^{5A} and R^{6A} are independently alkyl or alkylene-OC(═O)alkyl.

In some embodiments of formula (VII), R^{c} is OH or OC(═O)C₁₋₆ alkyl. In some embodiments, R¹⁰ is OH. In some embodiments, R^{c} is OC(═O)C₁₋₆ alkyl. In some embodiments, R^{c} is OC(═O)CH₃.

In some embodiments of formula (VII), R¹⁰ is NR^{5A}R^{6A}, 0-C₁₋₆ alkylene-OC(═O)C₁₋₆ alkyl, —O-C₁₋₅ alkylene-Si(C₁₋₆ alkyl)₂, 3-8 membered heterocycle or aryl. In some embodiments, R¹⁰ is NR^{5A}R^{6A}. In some embodiments, R^{5A} and R^{6A} are independently C₁₋₆ alkyl or C₁₋₆ alkylene-OC(═O) C₁₋₆ alkyl. In some embodiments, R¹⁰ is O-C₁-6 alkylene-OC(═O)C₁₋₆ alkyl. In some embodiments, R¹⁰ is —O-C₁₋₆ alkylene-Si(C₁₋₆ alkyl)₂. In some embodiments, R¹⁰ is 3-8 membered heterocycle. In some embodiments, R¹⁰ is aryl.

In some embodiments of formula (VII), R¹⁰ is

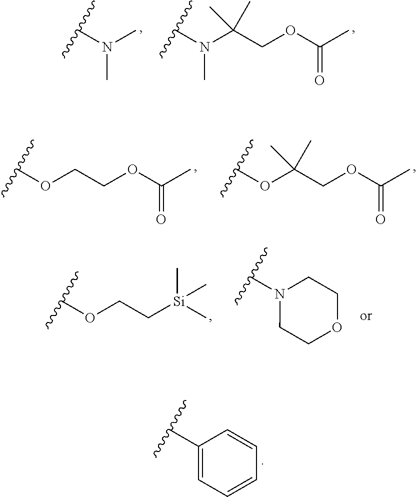

In some embodiments, of formula (VII), R^{c} is OH or OC(═O)CH₃, and

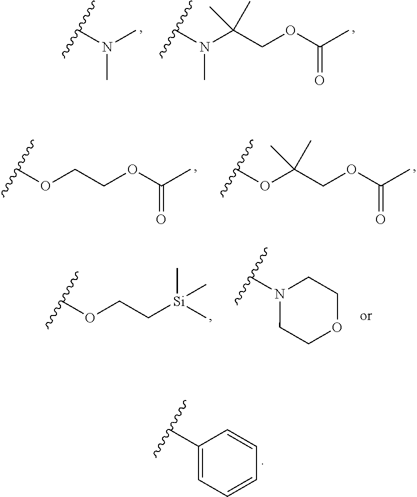

In some embodiments of formula (I), formula (II), formula (III), formula (IV), formula (V), formula (VI) or formula (VII), R² and R³ are independently C₁₋₆ alkyl.

In some embodiments of formula (I), formula (II), formula (III), formula (IV), formula (V), formula (VI) or formula (VII), R² and R³ are —CH₃.

In some embodiments of formula (I), formula (II), formula (III), formula (IV), formula (V), formula (VI) or formula (VII), R⁴ is H or C(═O)OC₁₋₆ alkyl.

In some embodiments of formula (I), formula (II), formula (III), formula (IV), formula (V), formula (VI) or formula (VII), R⁴ is H or C(═O)OCH₃.

In some embodiments of formula (I), formula (II), formula (III), formula (IV), formula (V), formula (VI) or formula (VII), R⁴ is H.

In some embodiments of formula (I), formula (II), formula (III), formula (IV), formula (V), formula (VI) or formula (VII), R² and R³ are independently CH₃, and R⁴ is H.

In some embodiments, this disclosure provides a compound of formula (I),

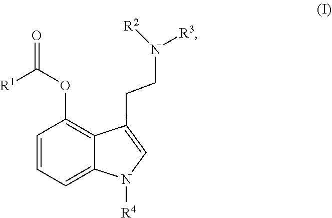

or pharmaceutically acceptable salt or deuterated form thereof,
   wherein:
R² and R³ are independently alkyl;
R⁴ is H or C(═O)Oalkyl; and
R¹ is defined according to i, ii or iii:
i. R¹ is —OR⁸, wherein R⁸ is —(CH₂CH₂O)ₘ-alkyl, m is 2, 3, 5 or 6;
ii. R¹ is —R^{9A}, wherein R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, arylene-NR⁵R⁶, or 5-membered heteroaryl substituted with 1-4 groups selected from alkyl, OH, halogen, haloalkyl, Oalkyl or O-haloalkyl;
   R⁵ and R⁶ are independently H, alkyl,

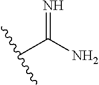 [SMILES: *C(=N)N]

or C(═O)alkyl wherein the alkyl is optionally substituted with NH₂, NH(alkyl) or N(alkyl)₂,
each R^{A} is independently H, halo, unsubstituted alky, or alkylene-OH,
or two R^{A} together with the carbon they are connected to form a cycloalkylene or heterocyclylene,
provided that at least one R^{A} is not H;
iii. R¹ is —(CH₂)ₙ—R^{9B}, wherein
   n is 1, 2, 3, 4, 5, or 6;
   R^{9B} is − branched alkylene-aryl wherein the aryl is substituted with 1-4 R^{7B},
   Si(alkyl)₃ or —(OCH₂CH₂)ₘ—Oalkyl;
   R^{7B} is —OP(═O)OH₂, C₁₋₆ alkyl,
   provided that when R^{9B} is Si(alkyl)₃ or

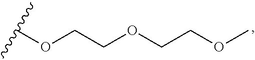 [SMILES: *OCCOCCOC]

then n is not 1.

In some embodiments of formula (I), when R^{9B} is Si(alkyl)₃ or

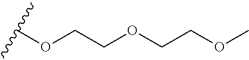 [SMILES: *OCCOCCOC]

then n is not 1.

In some embodiments, R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, or arylene-NR⁵R⁶, 5-membered heteroaryl comprising two N atoms wherein the heteroaryl is substituted with alkyl, OH, halogen, haloC₁₋₃alkyl, OC₁₋₃alkyl or O-haloC₁₋₃alkyl;
each R^{A} is independently H, halo, unsubstituted alky, or alkylene-OH, or
two R^{A} together with the carbon they are connected to form a C₃₋₈ cycloalkylene or 3-10 membered heterocyclylene,
provided that at least one R^{A} is not H.

In some embodiments, R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, 5-membered heteroaryl comprising two N atoms wherein the heteroaryl is substituted with C₁₋₃ alkyl, or arylene-NR⁵R⁶.

In embodiments, each R^{A} is independently H, halo, unsubstituted alky, or alkylene-OH, provided that at least one R^{A} is not H.

In some embodiments, the compound is a compound of Formula (II)

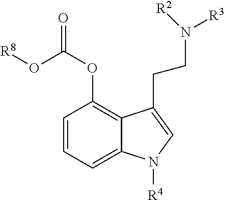

(II), or a pharmaceutically acceptable salt or deuterated form thereof,
R⁸ is —(CH₂CH₂O)ₘ-alkyl;
m is 2, 3, 5 or 6;
R² and R³ are independently alkyl;
R⁴ is H or C(═O)Oalkyl; and

In embodiments of R⁸, m is 2 or 3.

In embodiments, R⁸ is —(CH₂CH₂O)₂—CH₃ or —(CH₂CH₂O)₃—CH₃.

In embodiments, the compound is a compound of Formula (III)

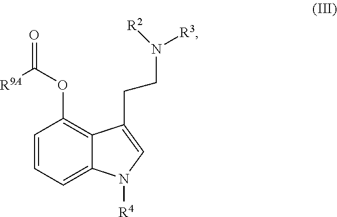

or a pharmaceutically acceptable salt or deuterated form thereof,
wherein:
R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, arylene-NR⁵R⁶, or 5-membered heteroaryl substituted with 1-4 groups selected from C₁₋₃ alkyl, OH or halogen;
   each R^{A} is independently H, halo, unsubstituted alky, or alkylene-OH, or
   two R^{A} together with the carbon they are connected to form a C₃₋₈ cycloalkylene or 3-10 membered heterocyclylene,
   provided that at least one R^{A} is not H;
   R² and R³ are independently alkyl;
   R⁴ is H or C(═O)Oalkyl,
      R⁵ and R⁶ are independently H, alkyl,

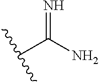 [SMILES: *C(=N)N]

or C(═O)alkyl wherein the alkyl is optionally substituted with NH₂, NH(alkyl) or N(alkyl)₂,

In some embodiments, wherein R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, 5-membered heteroaryl comprising two N atoms wherein the heteroaryl is substituted with C₁₋₃ alkyl, or arylene-NR⁵R⁶;
each R^{A} is independently H, halo, unsubstituted alky, or alkylene-OH, provided that at least one R^{A} is not H.

In some embodiments, R^{9A} is —C(R^{A})₂—O-C₁₋₆ alkyl, —C(R^{A})₂—O-C₃₋₆ cycloalkyl, cyclopropyl substituted with OC₁₋₆ alkyl, heterocyclyl substituted with C(═O)OC₁₋₆alkyl, 5-membered heteroaryl comprising two N atoms wherein the heteroaryl is substituted with C₁₋₃ alkyl, or arylene-NR⁵R⁶; each R^{A} is independently H or unsubstituted alkyl, provided that at least one R^{A} is not H,
R⁵ and R⁶ are independently H, alkyl, or C(═O)alkyl;
R^{7A} is OH, halo, alkyl, or haloalkyl.

In some embodiments, R^{9A} is 5-membered heteroaryl comprising two N atoms wherein the heteroaryl is substituted with C₁₋₃ alkyl, wherein the 5-membered heteroaryl is imidazole or pyrazole. In some embodiments, R^{9A} is pyrazole substituted with C₁₋₃ alkyl.

In some embodiments, R^{9A} is

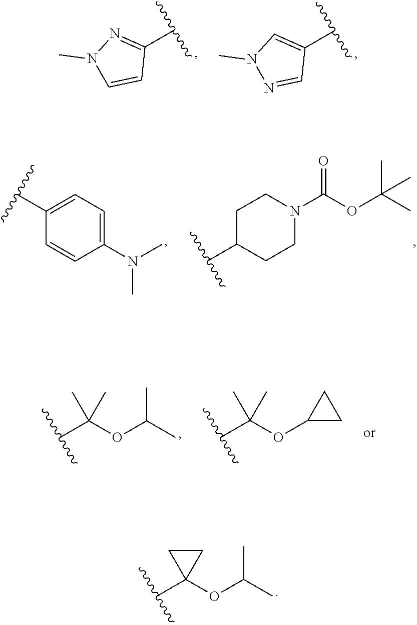

In some embodiments, R^{9A} is

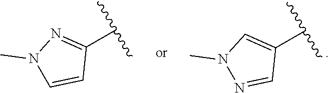

In some embodiments, the compound is a compound of Formula (IV)

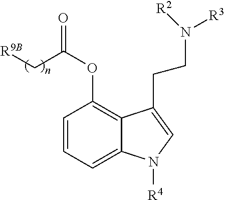

(IV), or a pharmaceutically acceptable salt or deuterated form thereof,
wherein:
n is 1, 2, 3, 4, 5, or 6;
R^{9B} is branched alkylene-aryl wherein the aryl is substituted with 1-4 R^{7B}, Si(alkyl)₃ or —(OCH₂CH₂)ₘ—Oalkyl;
m is 2 or 3;
R² and R³ are independently alkyl;
R⁴ is H or C(═O)Oalkyl;
R^{7B} is —OP(═O)OH₂ or C₁₋₆ alkyl;
provided that when R^{9B} is Si(alkyl)₃ or

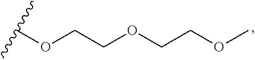 [SMILES: *OCCOCCOC]

then n is not 1.

In embodiments of formula (IV), when R^{9B} is Si(alkyl)₃ or

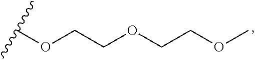 [SMILES: *OCCOCCOC]

then n is not 1.

In embodiments of formula (I) or (IV), n is 1, 2, or 3.

In embodiments, R^{9B} is

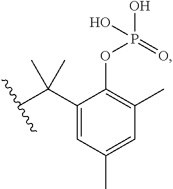 [SMILES: *C(C)(C)c1cc(C)cc(C)c1OP(=O)(O)O]

Si(CH₃)₃,

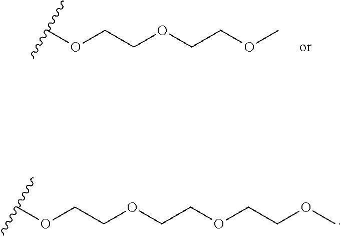

In embodiments of formula (I) or (IV), n is 1, 2, or 3.

In embodiments of formula (I) or (IV), n is 1, 2 or 3 and R^{9B}

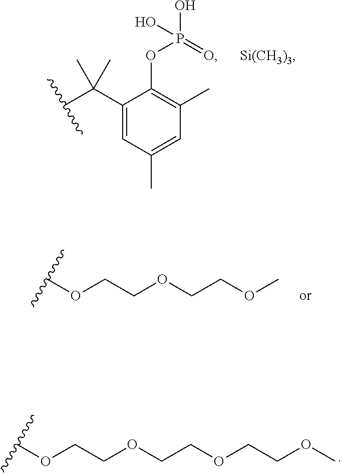

In some embodiments, the disclosure provides a compound of Formula (VII)

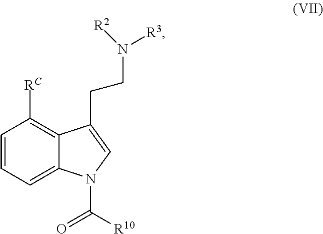

or a pharmaceutically acceptable salt or deuterated form thereof,
wherein:
R² and R³ are independently alkyl;
R^{c} is OH or OC(═O)alkyl;
R¹⁰ is aryl;
R^{5A} and R^{6A} are independently alkyl or alkylene-OC(═O)alkyl.

In some embodiments of formula (VII), R^{c} is OH, and R¹⁰ is phenyl.

In some embodiments, R² and R³ are independently C₁₋₆ alkyl.

In some embodiments, R² and R³ are —CH₃.

In some embodiments, R⁴ is H or C(═O)OC₁₋₆ alkyl.

In some embodiments, R⁴ is H or C(═O)OCH₃.

In some embodiments, R⁴ is H.

In some embodiments, R² and R³ are independently CH₃, and R⁴ is H.

In embodiments, the disclosure provides compounds of Table 1.

In embodiments, the disclosure provides compounds of Table 1 and Table 2.

TABLE 1

| Cmpd | | Formula |
|---|---|---|
| No. | Structure | No. |
| 101 | 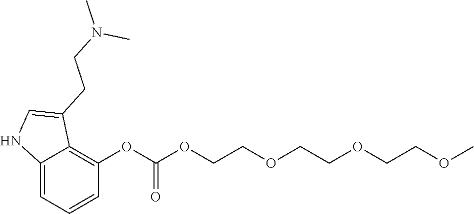 [SMILES: COCCOCCOCCOC(=O)Oc1cccc2[nH]cc(CCN(C)C)c12] | II |
| 102 | 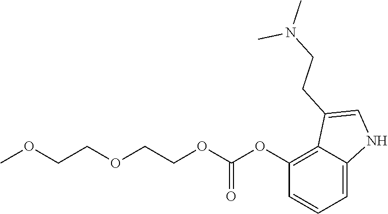 [SMILES: COCCOCCOC(=O)Oc1cccc2[nH]cc(CCN(C)C)c12] | II |
| 103 | 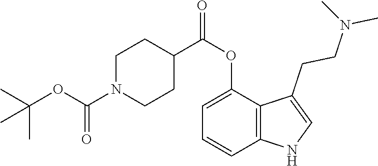 [SMILES: CN(C)CCc1c[nH]c2cccc(OC(=O)C3CCN(C(=O)OC(C)(C)C)CC3)c12] | III |
| 104 | 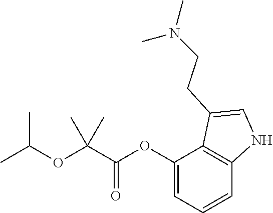 [SMILES: CC(C)OC(C)(C)C(=O)Oc1cccc2[nH]cc(CCN(C)C)c12] | III |
| 105 | 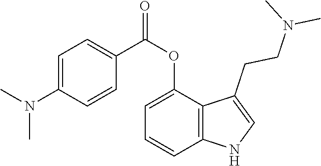 | III |
| 106 | 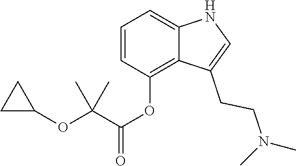 [SMILES: CN(C)CCc1c[nH]c2cccc(OC(=O)C(C)(C)OC3CC3)c12] | III |
| 107 | 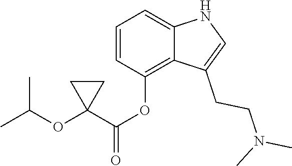 [SMILES: CC(C)OC1(C(=O)Oc2cccc3[nH]cc(CCN(C)C)c23)CC1] | III |
| 108 | 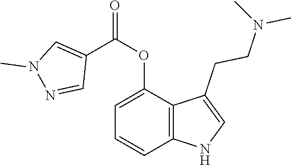 | III |
| 109 | 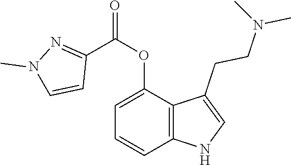 | III |
| 110 | 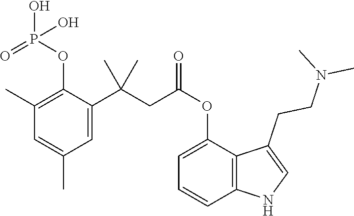 [SMILES: Cc1cc(C)c(OP(=O)(O)O)c(C(C)(C)CC(=O)Oc2cccc3[nH]cc(CCN(C)C)c23)c1] | IV |
| 111 | 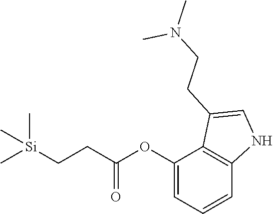 [SMILES: CN(C)CCc1c[nH]c2cccc(OC(=O)CC[Si](C)(C)C)c12] | IV |
| 112 | 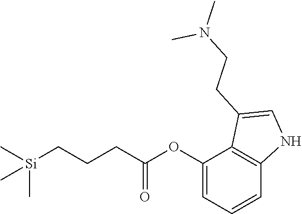 [SMILES: CN(C)CCc1c[nH]c2cccc(OC(=O)CCC[Si](C)(C)C)c12] | IV |
| 113 | 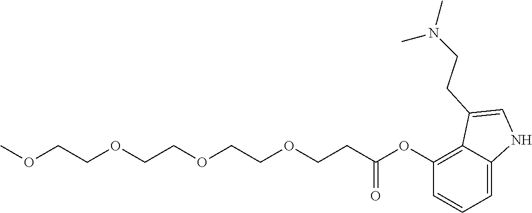 [SMILES: COCCOCCOCCOCCC(=O)Oc1cccc2[nH]cc(CCN(C)C)c12] | IV |
| 114 | 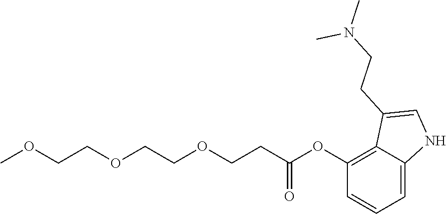 [SMILES: COCCOCCOCCC(=O)Oc1cccc2[nH]cc(CCN(C)C)c12] | IV |
| 115 | 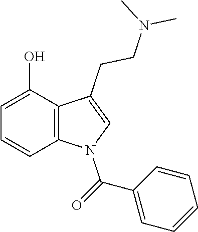 [SMILES: CN(C)CCc1cn(C(=O)c2ccccc2)c2cccc(O)c12] | VII |
| 184 | 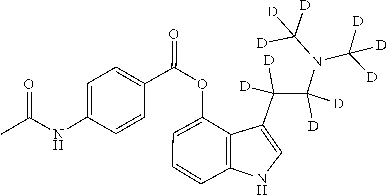 [SMILES: [2H]C([2H])([2H])N(C([2H])([2H])[2H])C([2H])([2H])C([2H])([2H])c1c[nH]c2cccc(OC(=O)c3ccc(NC(C)=O)cc3)c12] | III |
| 185 | 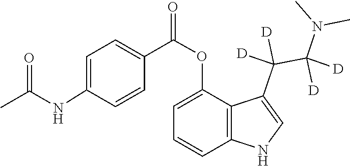 [SMILES: [2H]C([2H])(c1c[nH]c2cccc(OC(=O)c3ccc(NC(C)=O)cc3)c12)C([2H])([2H])N(C)C] | III |
| 186 | 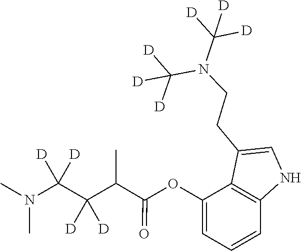 [SMILES: [2H]C([2H])([2H])N(CCc1c[nH]c2cccc(OC(=O)C(C)C([2H])([2H])C([2H])([2H])N(C)C)c12)C([2H])([2H])[2H]] | III |
| 187 | 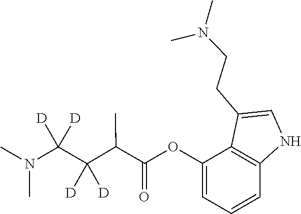 [SMILES: [2H]C([2H])(C(C)C(=O)Oc1cccc2[nH]cc(CCN(C)C)c12)C([2H])([2H])N(C)C] | III |
| 188 | 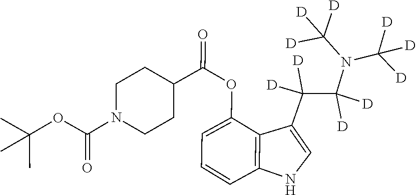 [SMILES: [2H]C([2H])([2H])N(C([2H])([2H])[2H])C([2H])([2H])C([2H])([2H])c1c[nH]c2cccc(OC(=O)C3CCN(C(=O)OC(C)(C)C)CC3)c12] | III |
| 189 | 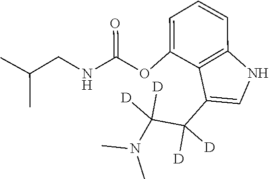 [SMILES: [2H]C([2H])(c1c[nH]c2cccc(OC(=O)NCC(C)C)c12)C([2H])([2H])N(C)C] | V |
| 190 | 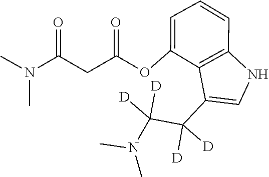 [SMILES: [2H]C([2H])(c1c[nH]c2cccc(OC(=O)CC(=O)N(C)C)c12)C([2H])([2H])N(C)C] | IV |
| 191 | 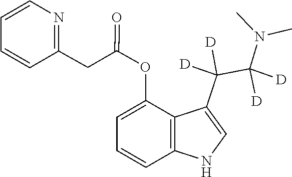 [SMILES: [2H]C([2H])(c1c[nH]c2cccc(OC(=O)Cc3ccccn3)c12)C([2H])([2H])N(C)C] | IV |
| 192 | 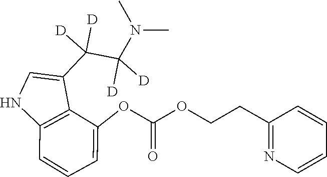 [SMILES: [2H]C([2H])(c1c[nH]c2cccc(OC(=O)OCCc3ccccn3)c12)C([2H])([2H])N(C)C] | II |
| 193 | 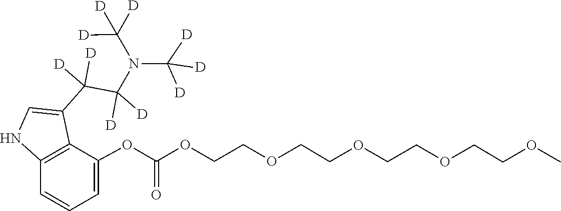 [SMILES: [2H]C([2H])([2H])N(C([2H])([2H])[2H])C([2H])([2H])C([2H])([2H])c1c[nH]c2cccc(OC(=O)OCCOCCOCCOCCOC)c12] | II |
| 194 | 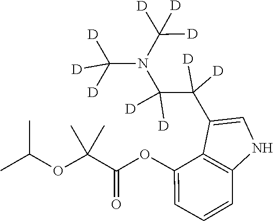 [SMILES: [2H]C([2H])([2H])N(C([2H])([2H])[2H])C([2H])([2H])C([2H])([2H])c1c[nH]c2cccc(OC(=O)C(C)(C)OC(C)C)c12] | III |

TABLE 2

| Compound | | Formula |
|---|---|---|
| No. | Structure | No |
| 116 | 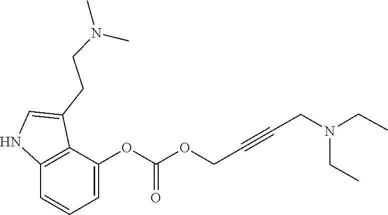 [SMILES: CCN(CC)CC#CCOC(=O)Oc1cccc2[nH]cc(CCN(C)C)c12] | II |
| 117 | 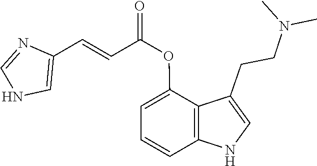 [SMILES: CN(C)CCc1c[nH]c2cccc(OC(=O)/C=C/c3c[nH]cn3)c12] | III |
| 118 | 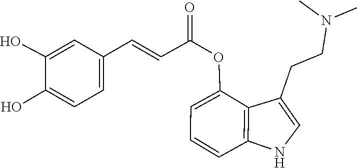 [SMILES: CN(C)CCc1c[nH]c2cccc(OC(=O)/C=C/c3ccc(O)c(O)c3)c12] | III |
| 119 | 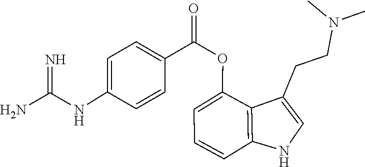 [SMILES: CN(C)CCc1c[nH]c2cccc(OC(=O)c3ccc(NC(=N)N)cc3)c12] | III |
| 120 | 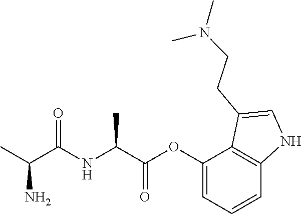 [SMILES: C[C@H](N)C(=O)N[C@@H](C)C(=O)Oc1cccc2[nH]cc(CCN(C)C)c12] | III |
| 121 | 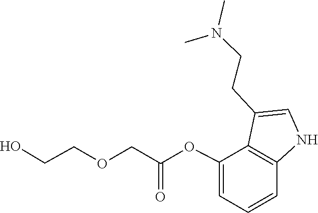 [SMILES: CN(C)CCc1c[nH]c2cccc(OC(=O)COCCO)c12] | IV |
| 122 | 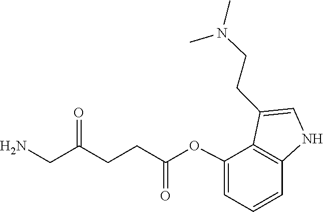 [SMILES: CN(C)CCc1c[nH]c2cccc(OC(=O)CCC(=O)CN)c12] | IV |
| 123 | 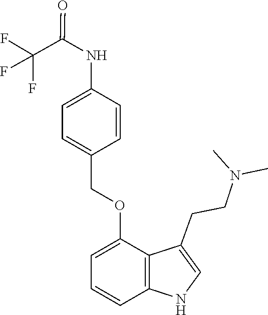 [SMILES: CN(C)CCc1c[nH]c2cccc(OCc3ccc(NC(=O)C(F)(F)F)cc3)c12] | VI |
| 124 | 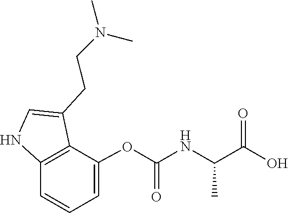 [SMILES: C[C@H](NC(=O)Oc1cccc2[nH]cc(CCN(C)C)c12)C(=O)O] | V |
| 125 | 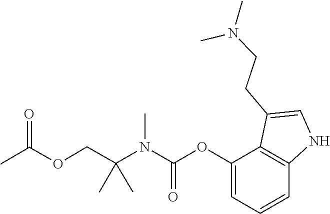 [SMILES: CC(=O)OCC(C)(C)N(C)C(=O)Oc1cccc2[nH]cc(CCN(C)C)c12] | V |
| 126 | 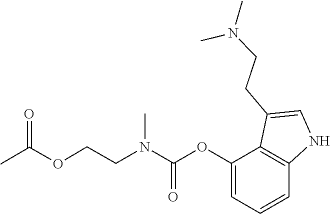 [SMILES: CC(=O)OCCN(C)C(=O)Oc1cccc2[nH]cc(CCN(C)C)c12] | V |
| 127 | 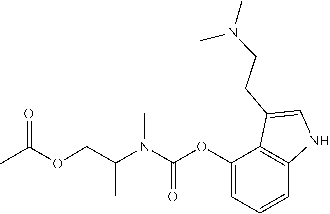 [SMILES: CC(=O)OCC(C)N(C)C(=O)Oc1cccc2[nH]cc(CCN(C)C)c12] | V |
| 128 | 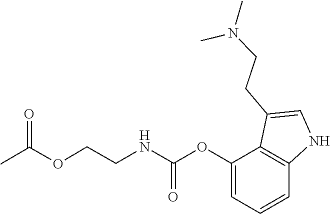 [SMILES: CC(=O)OCCNC(=O)Oc1cccc2[nH]cc(CCN(C)C)c12] | V |
| 129 | 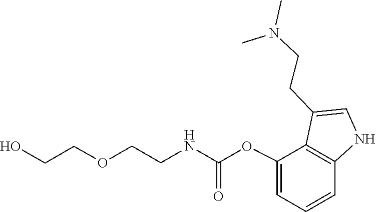 [SMILES: CN(C)CCc1c[nH]c2cccc(OC(=O)NCCOCCO)c12] | V |
| 130 | 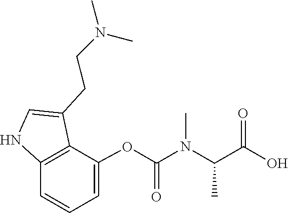 [SMILES: C[C@@H](C(=O)O)N(C)C(=O)Oc1cccc2[nH]cc(CCN(C)C)c12] | V |
| 131 | 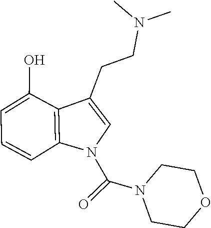 [SMILES: CN(C)CCc1cn(C(=O)N2CCOCC2)c2cccc(O)c12] | VII |
| 132 | 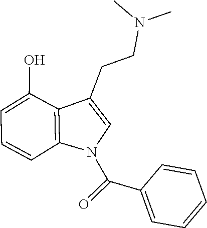 [SMILES: CN(C)CCc1cn(C(=O)c2ccccc2)c2cccc(O)c12] | VII |
| 133 | 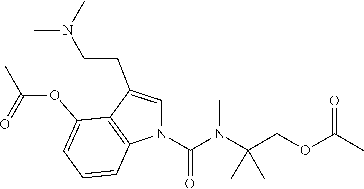 [SMILES: CC(=O)OCC(C)(C)N(C)C(=O)n1cc(CCN(C)C)c2c(OC(C)=O)cccc21] | VII |
| 134 | 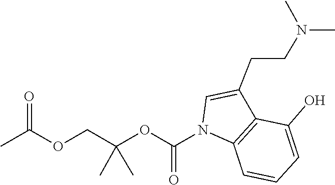 [SMILES: CC(=O)OCC(C)(C)OC(=O)n1cc(CCN(C)C)c2c(O)cccc21] | VII |
| 135 | 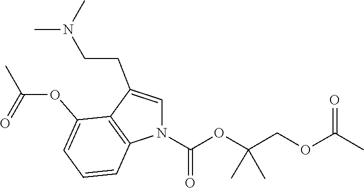 [SMILES: CC(=O)OCC(C)(C)OC(=O)n1cc(CCN(C)C)c2c(OC(C)=O)cccc21] | VII |
| 136 | 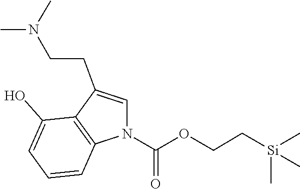 | VII |
| 137 | 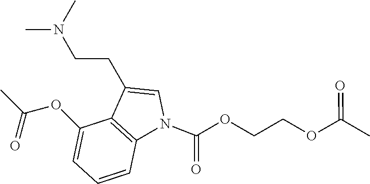 [SMILES: CC(=O)OCCOC(=O)n1cc(CCN(C)C)c2c(OC(C)=O)cccc21] | VII |
| 138 | 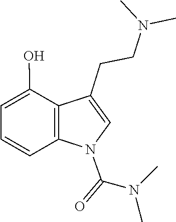 [SMILES: CN(C)CCc1cn(C(=O)N(C)C)c2cccc(O)c12] | VII |

TABLE 3

| cmpd | | Formula |
|---|---|---|
| No. | Structure | No. |
| 141 | 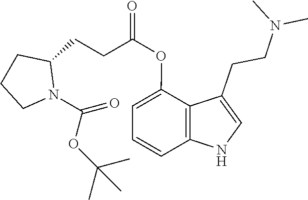 [SMILES: CN(C)CCc1c[nH]c2cccc(OC(=O)CC[C@H]3CCCN3C(=O)OC(C)(C)C)c12] | IV |
| 142 | 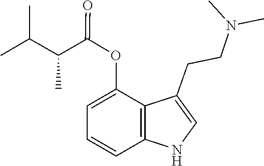 [SMILES: CC(C)[C@@H](C)C(=O)Oc1cccc2[nH]cc(CCN(C)C)c12] | III |
| 143 | 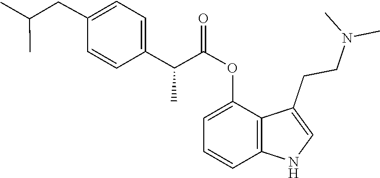 [SMILES: CC(C)Cc1ccc([C@@H](C)C(=O)Oc2cccc3[nH]cc(CCN(C)C)c23)cc1] | III |
| 144 | 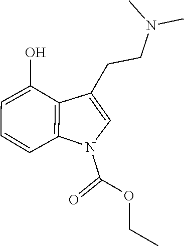 [SMILES: CCOC(=O)n1cc(CCN(C)C)c2c(O)cccc21] | VII |
| 145 | 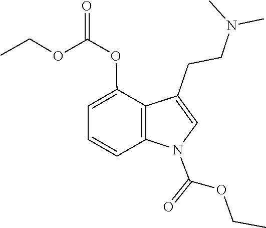 [SMILES: CCOC(=O)Oc1cccc2c1c(CCN(C)C)cn2C(=O)OCC] | II |
| 146 | 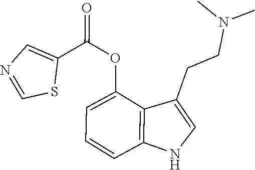 [SMILES: CN(C)CCc1c[nH]c2cccc(OC(=O)c3cncs3)c12] | III |
| 147 | 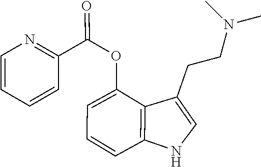 [SMILES: CN(C)CCc1c[nH]c2cccc(OC(=O)c3ccccn3)c12] | III |
| 148 | 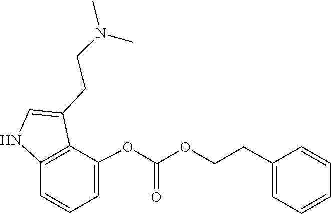 [SMILES: CN(C)CCc1c[nH]c2cccc(OC(=O)OCCc3ccccc3)c12] | II |
| 149 | 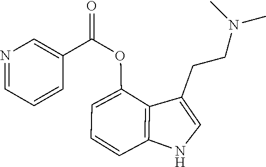 [SMILES: CN(C)CCc1c[nH]c2cccc(OC(=O)c3cccnc3)c12] | III |
| 150 | 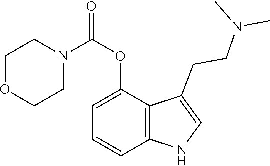 [SMILES: CN(C)CCc1c[nH]c2cccc(OC(=O)N3CCOCC3)c12] | VI |
| 151 | 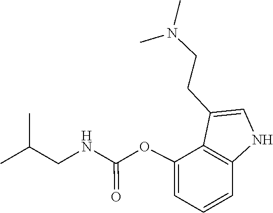 [SMILES: CC(C)CNC(=O)Oc1cccc2[nH]cc(CCN(C)C)c12] | VI |
| 152 | 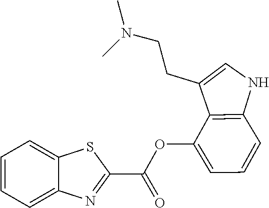 [SMILES: CN(C)CCc1c[nH]c2cccc(OC(=O)c3nc4ccccc4s3)c12] | III |
| 153 | 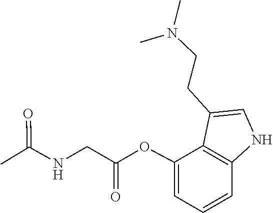 [SMILES: CC(=O)NCC(=O)Oc1cccc2[nH]cc(CCN(C)C)c12] | IV |
| 154 | 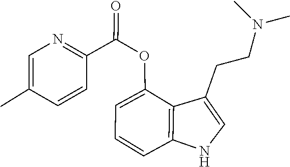 | III |
| 155 | 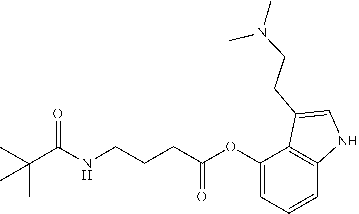 [SMILES: CN(C)CCc1c[nH]c2cccc(OC(=O)CCCNC(=O)C(C)(C)C)c12] | IV |
| 156 | 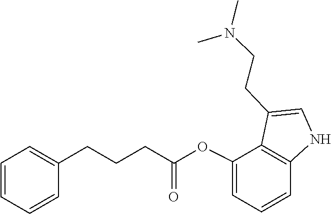 [SMILES: CN(C)CCc1c[nH]c2cccc(OC(=O)CCCc3ccccc3)c12] | IV |
| 157 | 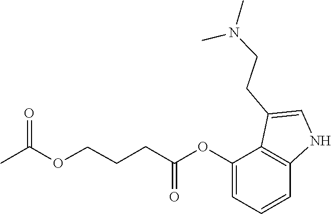 [SMILES: CC(=O)OCCCC(=O)Oc1cccc2[nH]cc(CCN(C)C)c12] | IV |
| 158 | 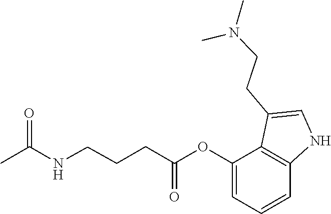 [SMILES: CC(=O)NCCCC(=O)Oc1cccc2[nH]cc(CCN(C)C)c12] | IV |
| 159 | 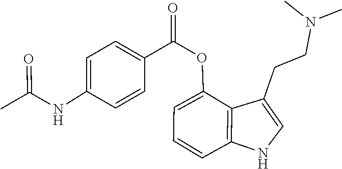 [SMILES: CC(=O)Nc1ccc(C(=O)Oc2cccc3[nH]cc(CCN(C)C)c23)cc1] | III |
| 160 | 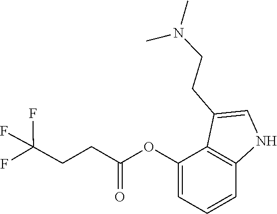 [SMILES: CN(C)CCc1c[nH]c2cccc(OC(=O)CCC(F)(F)F)c12] | IV |
| 161 | 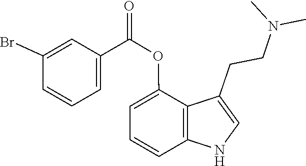 [SMILES: CN(C)CCc1c[nH]c2cccc(OC(=O)c3cccc(Br)c3)c12] | III |
| 162 | 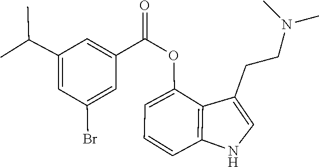 [SMILES: CC(C)c1cc(Br)cc(C(=O)Oc2cccc3[nH]cc(CCN(C)C)c23)c1] | III |
| 163 | 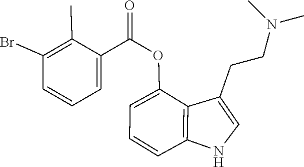 [SMILES: Cc1c(Br)cccc1C(=O)Oc1cccc2[nH]cc(CCN(C)C)c12] | III |
| 164 | 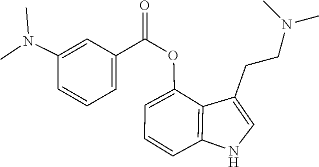 | III |
| 165 | 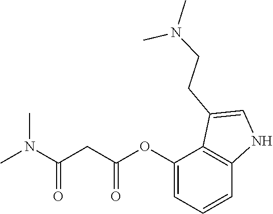 [SMILES: CN(C)CCc1c[nH]c2cccc(OC(=O)CC(=O)N(C)C)c12] | IV |
| 166 | 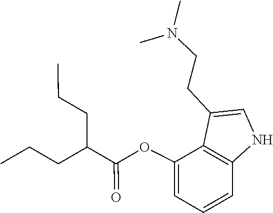 [SMILES: CCCC(CCC)C(=O)Oc1cccc2[nH]cc(CCN(C)C)c12] | III |
| 167 | 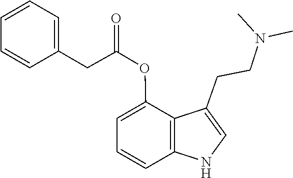 [SMILES: CN(C)CCc1c[nH]c2cccc(OC(=O)Cc3ccccc3)c12] | IV |
| 168 | 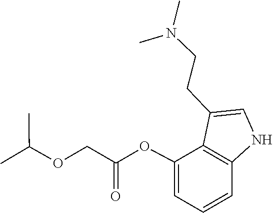 [SMILES: CC(C)OCC(=O)Oc1cccc2[nH]cc(CCN(C)C)c12] | IV |
| 169 | 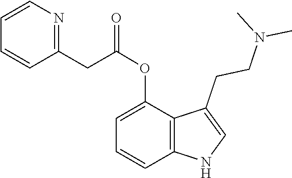 [SMILES: CN(C)CCc1c[nH]c2cccc(OC(=O)Cc3ccccn3)c12] | IV |
| 170 | 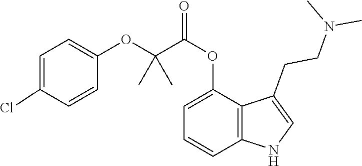 [SMILES: CN(C)CCc1c[nH]c2cccc(OC(=O)C(C)(C)Oc3ccc(Cl)cc3)c12] | III |
| 171 | 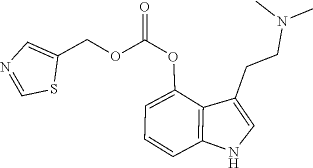 [SMILES: CN(C)CCc1c[nH]c2cccc(OC(=O)OCc3cncs3)c12] | II |
| 172 | 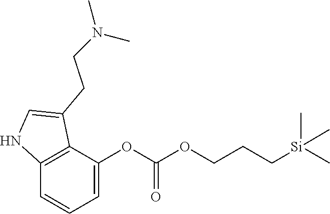 [SMILES: CN(C)CCc1c[nH]c2cccc(OC(=O)OCCC[Si](C)(C)C)c12] | II |
| 173 | 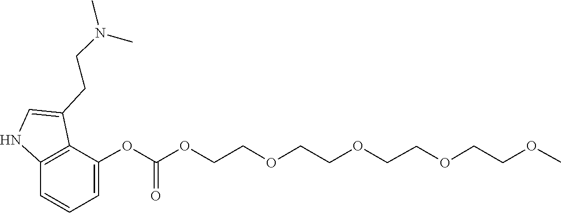 [SMILES: COCCOCCOCCOCCOC(=O)Oc1cccc2[nH]cc(CCN(C)C)c12] | II |
| 174 | 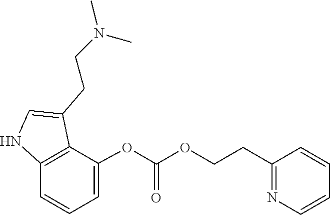 [SMILES: CN(C)CCc1c[nH]c2cccc(OC(=O)OCCc3ccccn3)c12] | II |
| 175 | 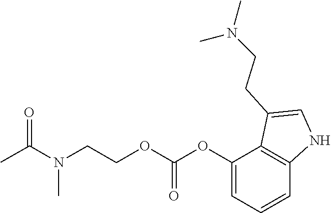 [SMILES: CC(=O)N(C)CCOC(=O)Oc1cccc2[nH]cc(CCN(C)C)c12] | II |
| 176 | 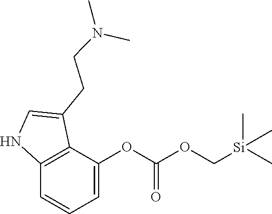 [SMILES: CN(C)CCc1c[nH]c2cccc(OC(=O)OC[Si](C)(C)C)c12] | II |
| 177 | 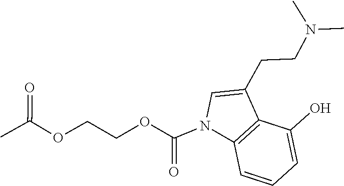 [SMILES: CC(=O)OCCOC(=O)n1cc(CCN(C)C)c2c(O)cccc21] | VII |
| 178 | 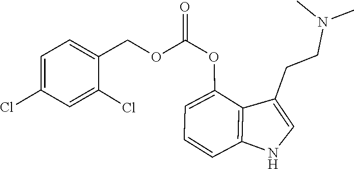 [SMILES: CN(C)CCc1c[nH]c2cccc(OC(=O)OCc3ccc(Cl)cc3Cl)c12] | II |
| 179 | 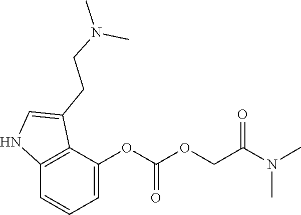 [SMILES: CN(C)CCc1c[nH]c2cccc(OC(=O)OCC(=O)N(C)C)c12] | II |
| 180 | 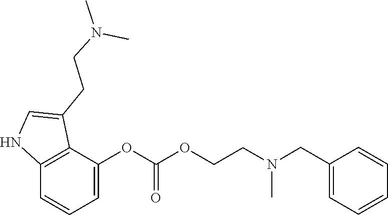 [SMILES: CN(C)CCc1c[nH]c2cccc(OC(=O)OCCN(C)Cc3ccccc3)c12] | II |
| 182 | 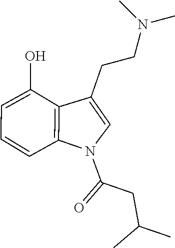 [SMILES: CC(C)CC(=O)n1cc(CCN(C)C)c2c(O)cccc21] | VII |

### Pharmaceutical Compositions

The present disclosure provides pharmaceutical compositions comprising at least one compound disclosed herein and one or more pharmaceutically acceptable excipients.

The compounds provided herein may be administered as compounds per se or may be formulated as pharmaceutical compositions. The pharmaceutical compositions may comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

### Methods of Treatment

The disclosure further relates to compounds disclosed herein, or a pharmaceutical composition comprising at least one compound disclosed here, for use in the treatment of a serotonin 5-HT2A receptor associated disease/disorder. In embodiments, the compounds may be used in the treatment of an anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), and premenstrual syndrome (PMS).

### Numbered Embodiments

1. A compound of Formula (I)

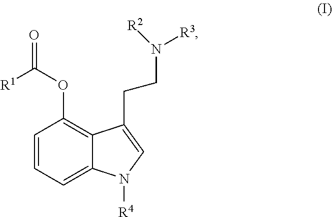

or pharmaceutically acceptable salt or deuterated form thereof,
   wherein:
R² and R³ are independently alkyl;
R⁴ is H or C(═O)Oalkyl; and
R¹ is defined according to i, ii, iii, or iv:
   i. R¹ is —OR⁸, wherein R⁸ is alkynyl substituted with NR⁵R⁶, or —(CH₂CH₂O)ₘ alkyl, m is 1, 2, 3, 5 or 6;
      R⁵ and R⁶ are independently H, alkyl, C(═O)alkyl, alkylene-aryl, or S(O)₂alkyl;
   ii. R¹ is —R^{9A}, wherein R^{9A} is —C(R^{A})₂NR⁵R⁶, —C(R^{A})₂—O-alkyl, —C(R^{A})₂—O-cycloalkyl, cycloalkyl substituted with Oalkyl, heterocyclyl substituted with C(═O)Oalkyl, alkenylene-aryl wherein the aryl is substituted with 1-4 R⁷, alkenylene-heteroaryl wherein the heteroaryl is substituted with 0-4 R⁷, aryl substituted with NR⁵R⁶, or 5-membered heteroaryl substituted with 1-4 groups selected from alkyl, OH, halogen, haloalkyl, Oalkyl or O-haloalkyl;
      each R^{A} is independently H, halo, unsubstituted alkyl, or alkylene-OH, or
      two R^{A} together with the carbon they are connected to form a cycloalkylene or heterocyclylene,
      provided that at least one R^{A} is not H;
      R⁵ and R⁶ are independently H, alkyl, or

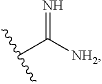 [SMILES: *C(=N)N]

or C(═O)alkyl wherein the alkyl is optionally substituted with NH₂, NH(alkyl), N(alkyl)₂, C(═O)aryl, C(═O)Oalkyl, S(O)₂alkyl, or alkylene-heterocyclyl;
R⁷ is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl.
iii. R¹ is —(CH₂)ₙ—R^{9B}, wherein
   n is 1, 2, 3, 4, 5, or 6;
   R^{9B} is —O-alkylene-OH, —C(═O)-alkylene-NR⁵R⁶, alkylene-aryl wherein the aryl is substituted with 1-4 R⁷, Si(alkyl)₃ or —(OCH₂CH₂)ₘ—Oalkyl, m is 2 or 3, provided that when R^{9B} is Si(alkyl)₃, then n is not 1;
   R⁵ and R⁶ are independently H, alkyl, C(═O)alkyl, C(═O)aryl, C(═O)Oalkyl, S(O)₂alkyl, alkylene-heterocyclyl;
   R⁷ is —OP(═O)OH₂, C₁₋₆ alkyl;
iv. R¹ is —N(R^{5A})(R^{6A}), wherein
   R^{5A} and R^{6A} are independently H, C₁₋₆ alkyl optionally substituted with —COOH, —OC(═O)alkyl or —O-alkylene-OH,
   provided that when one of R^{5A} and R^{6A} is H, the other cannot be H, or CH₂CH(CH₃)₂, and
   further provided that when one of R^{5A} and R^{6A} is CH₃, the other cannot be H, CH₃ or CH₂CH₃ or CH₂CH(CH₃)₂.

2. The compound of embodiment 1, wherein the compound is a compound of Formula (II)

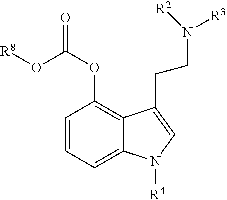

(II), or a pharmaceutically acceptable salt or deuterated form thereof,
   wherein:
R⁸ is alkynylene-NR⁵R⁶ or —(CH₂CH₂O)ₘ-alkyl;
m is 1, 2, 3, 5 or 6;
R² and R³ are independently alkyl;
R⁴ is H or C(═O)Oalkyl; and
R⁵ and R⁶ are independently H, alkyl, C(═O)H, C(═O)alkyl, alkylene-heteroaryl, alkylene-aryl, alkylene-carbocyclyl, alkylene-heterocyclyl, S(═O)alkyl, or S(═O)₂alkyl.

3. The compound of embodiment 1 or 2, wherein R⁸ is:
C₁₋₆ alkynyl substituted with N(C₁₋₆ alkyl)₂, or
—(CH₂CH₂O)ₘ—CH₃, wherein m is 1, 2 or 3.

4. The compound of any one of embodiment 1-3, wherein R⁸ is,

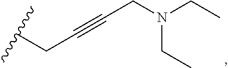 [SMILES: *CC#CCN(CC)CC]

—(CH₂CH₂O)—CH₃, —(CH₂CH₂O)₂—CH₃ or —(CH₂CH₂O)₃—CH₃.

5. The compound of embodiment 1, wherein the compound is a compound of Formula (III)

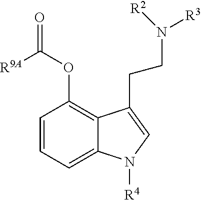

(III), or a pharmaceutically acceptable salt or deuterated form thereof,
   wherein:
R^{9A} is —C(R^{A})₂NR⁵R⁶, —C(R^{A})₂—O-alkyl, —C(R^{A})₂—O-cycloalkyl, cycloalkyl substituted with Oalkyl, heterocyclyl substituted with C(═O)Oalkyl, alkenylene-aryl wherein the aryl is substituted with 1-4 R⁷, alkenylene-heteroaryl wherein the heteroaryl is substituted with 0-4 R⁷, aryl substituted with NR⁵R⁶ or 5-membered heteroaryl substituted with 1-4 groups selected from alkyl, OH, halogen, haloalkyl, Oalkyl or O-haloalkyl;
each R^{A} is independently H, halo, unsubstituted alkyl, or alkylene-OH, or
two R^{A} together with the carbon they are connected to form a cycloalkylene or heterocyclylene,
provided that at least one R^{A} is not H;
R² and R³ are independently alkyl;
R⁴ is H or C(═O)Oalkyl;
R⁵ and R⁶ are independently H, alkyl,

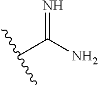 [SMILES: *C(=N)N]

or C(═O)alkyl wherein the alkyl is optionally substituted with NH₂, NH(alkyl) or N(alkyl)₂;
R⁷ is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl.

6. The compound of embodiment 5, wherein
R^{9A} is selected from —C(R^{A})₂NR⁵R⁶, —C(R^{A})₂—O-C₁₋₆ alkyl, —C(R^{A})₂—O-C₃₋₆cycloalkyl, cyclopropyl substituted with OC₁₋₆ alkyl, heterocyclyl substituted with C(═O)OC₁₋₆alkyl, C₁₋₆ alkenylene-aryl wherein the aryl is substituted with 1-4 R⁷, C₁₋₆ alkenylene-heteroaryl,arylene-NR⁵R⁶, or 5-membered heteroaryl comprising two N atoms wherein the heteroaryl is substituted with C₁₋₃ alkyl;
each R^{A} is independently H or unsubstituted alkyl, provided that at least one R^{A} is not H,
R² and R³ are independently alkyl;
R⁴ is H or C(═O)Oalkyl;
R⁵ and R⁶ are independently H, alkyl,

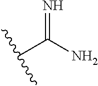 [SMILES: *C(=N)N]

or C(═O)alkyl wherein the alkyl is optionally substituted with NH₂, NH(alkyl) or N(alkyl)₂;
R⁷ is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl.

7. The compound of any one of embodiments 1 or 5-6, wherein R^{9A} is

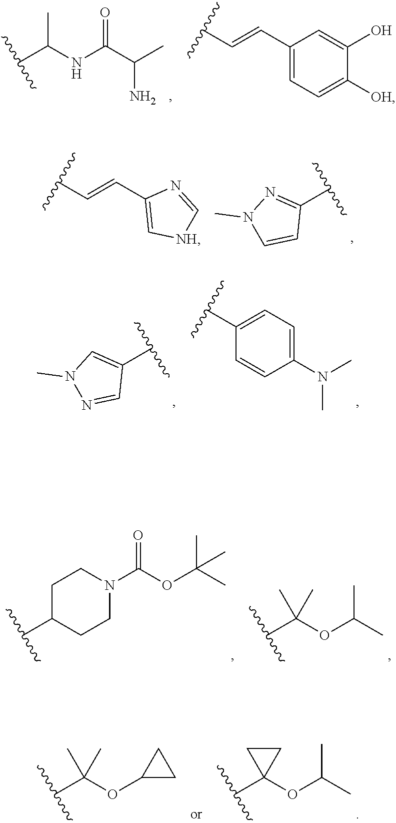

8. The compound of embodiment 1, wherein the compound is a compound of Formula (IV)

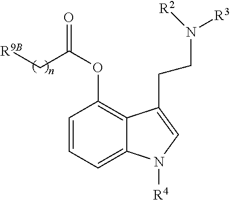

(IV), or a pharmaceutically acceptable salt or deuterated form thereof,
   wherein:
n is 1, 2, 3, 4, 5, or 6;
R^{9B} is —O-alkylene-OH, —C(═O)-alkylene-NR⁵R⁶, alkylene-aryl wherein the aryl is substituted with 1-4 R⁷, Si(alkyl)₃ or —(OCH₂CH₂)ₘ—Oalkyl;
m is 2 or 3;
R² and R³ are independently alkyl;
R⁴ is H or C(═O)Oalkyl;
R⁵ and R⁶ are independently H, alkyl, C(═O)alkyl, C(═O)aryl, C(═O)Oalkyl, S(O)₂alkyl or alkylene-heterocyclyl;
R⁷ is —OP(═O)OH₂, C₁₋₆ alkyl,
provided that when R^{9B} is Si(alkyl)₃, then n is not 1.

9. The compound of any one of embodiments 1 or 8, wherein
n is 1, 2 or 3;
R^{9B} is —O-C₁₋₆ alkylene-OH or —C(═O)—C₁₋₆ alkylene-NR⁵R⁶, C₁₋₆ alkylene-aryl wherein the aryl is substituted with 1-4 R⁷, Si(C₁₋₆ alkyl)₃, —(OCH₂CH₂)ₘ—Oalkyl;
m is 2 or 3;
R² and R³ are independently alkyl;
R⁴ is H or C(═O)OC₁₋₆ alkyl;
R⁵ and R⁶ are independently H or alkyl.

10. The compound of any one of embodiments 1 or 8-9, wherein R^{9B} is

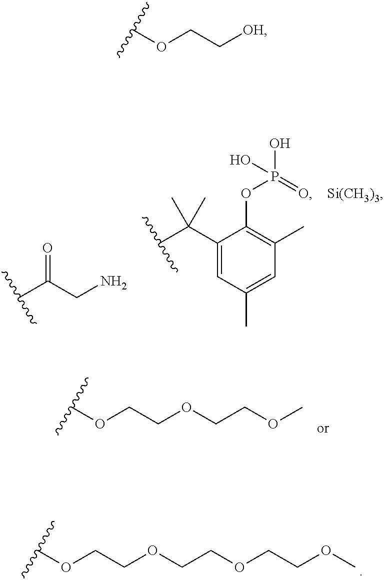

11. The compound of any one of embodiments 1 or 8-10, wherein n is 1, 2 or 3, and R^{9B} is

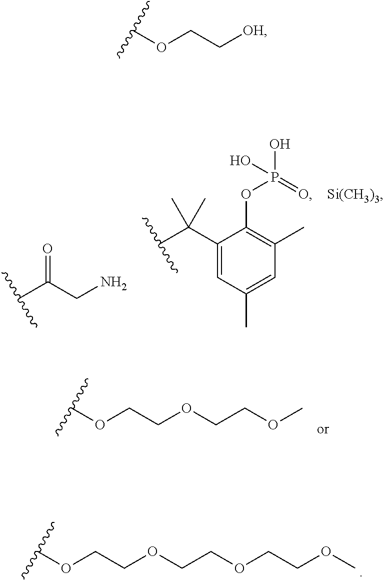

12. The compound of embodiment 1, wherein the compound is a compound of Formula (V)

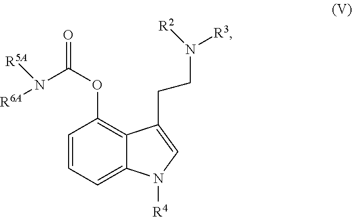

or a pharmaceutically acceptable salt or deuterated form thereof,
   wherein:
R² and R³ are independently alkyl;
R⁴ is H or C(═O)Oalkyl;
R^{5A} and R^{6A} are independently H, C₁₋₆ alkyl optionally substituted with —COOH, —OC(═O)alkyl or —O-alkylene-OH,
provided that both R^{5A} and R^{6A} are not H, and when one of R^{5A} and R^{6A} is H, the other cannot be H, or CH₂CH(CH₃)₂, and
further provided that when one of R^{5A} and R^{6A} is CH₃, the other cannot be H, CH₃ or CH₂CH₃ or CH₂CH(CH₃)₂.

13. The compound of claim 1 or 12, wherein R^{5A} and R^{6A} are independently H, CH₃,

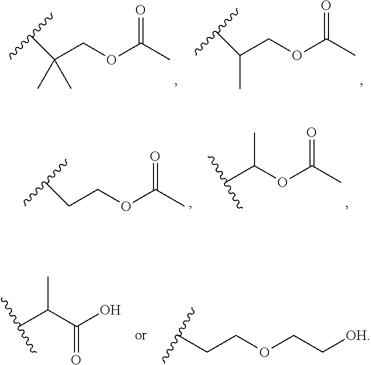

14. A compound of Formula (VI)

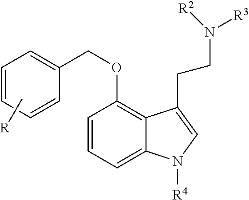

(V), or a pharmaceutically acceptable salt or deuterated thereof,
   wherein:
R is NR⁵R⁶;
R² and R³ are independently alkyl;
R⁴ is H or C(═O)Oalkyl;
R⁵ and R⁶ are independently H, alkyl, haloalkyl, or C(═O)haloalkyl.

15. The compound of embodiment 14, wherein R⁵ is H and R⁶ is C(═O)CF₃.

16. A compound of Formula (VII)

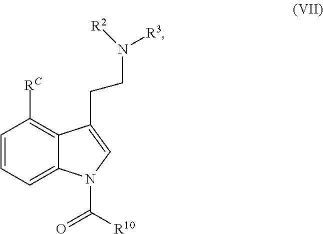

or a pharmaceutically acceptable salt or deuterated form thereof,
   wherein:
R² and R³ are independently alkyl;
R^{c} is OH or OC(═O)alkyl;
R¹⁰ is NR^{9A}R^{6A}, O-alkylene-OC(═O)alkyl, O-alkylene-Si(alkyl)₂, heterocycle, or aryl;
R^{5A} and R^{6A} are independently alkyl or alkylene-OC(═O)alkyl.

17. The compound of embodiment 16, wherein
R^{c} is OH or OC(═O)C₁₋₆ alkyl;
R¹⁰ is NR^{5A}R^{6A}, 0-C₁₋₆ alkylene-OC(═O)C₁₋₆ alkyl, —O-C₁₋₆ alkylene-Si(C₁₋₆ alkyl)₂, 3-8 membered heterocycle or aryl;
R^{5A} and R^{6A} are independently C₁₋₆ alkyl or C₁₋₆ alkylene-OC(═O) C₁₋₆ alkyl.

18. The compound of embodiment 16 or 17, wherein
R^{c} is OH or OC(═O)CH₃, and

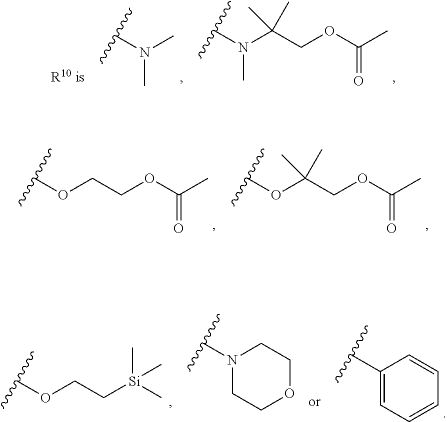

19. The compound of any one of embodiments 1-18, wherein R² and R³ are independently C₁₋₆ alkyl.

20. The compound of any one of embodiments 1-19, wherein R² and R³ are —CH₃.

21. The compound of any one of embodiments 1-20, wherein R⁴ is H or C(═O)OC₁₋₆ alkyl.

22. The compound of any one of embodiments 1-21, wherein R⁴ is H or C(═O)OCH₃.

23. The compound of any one of embodiments 1-22, wherein R⁴ is H.

24. The compound of any one of embodiments 1-23, wherein R² and R³ are independently CH₃, and R⁴ is H.

25. A compound selected from Table 1, Table 2, a pharmaceutically acceptable salt thereof, or a deuterated form thereof.

26. A pharmaceutical composition comprising a compound of embodiment 1-25, or a pharmaceutically acceptable salt thereof.

27. A method of treating a disease comprising administrating a pharmaceutical composition of embodiment 26.

28. A method of treating a disease comprising subcutaneously administrating a pharmaceutical composition of embodiment 26.

29. A method of treating a disease comprising subcutaneously administrating a pharmaceutical composition comprising a compound selected from Table 1, Table 2, Table 3, a pharmaceutically acceptable salt thereof, or a deuterated form thereof.

30. The method of any one of embodiments 27-29, wherein the disease is a 5-HT2A receptor associated disease or disorder.

31. The method of embodiment 30, wherein the neuropsychiatric disease is selected from anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, anxiety disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), and premenstrual syndrome (PMS).

32. A compound of Formula (I)

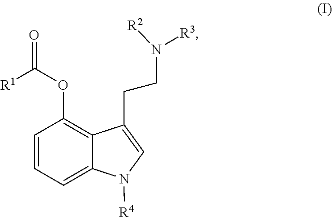

or pharmaceutically acceptable salt or deuterated form thereof,
   wherein:
R² and R³ are independently alkyl;
R⁴ is H or C(═O)Oalkyl; and
R¹ is defined according to i, ii or iii:
i. R¹ is —OR⁸, wherein R⁸ is —(CH₂CH₂O)ₘ-alkyl, m is 2, 3, 5 or 6;
ii. R¹ is —R^{9A}, wherein R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, arylene-NR⁵R⁶, or 5-membered heteroaryl substituted with 1-4 groups selected from alkyl, OH, halogen, haloalkyl, Oalkyl or O-haloalkyl;
each R^{A} is independently H, halo, unsubstituted alkyl, or alkylene-OH, or
two R^{A} together with the carbon they are connected to form a cycloalkylene or heterocyclylene,
provided that at least one R^{A} is not H;
iii. R¹ is —(CH₂)ₙ—R^{9B}, wherein
n is 1, 2, 3, 4, 5, or 6;
R^{9B} is − branched alkylene-aryl wherein the aryl is substituted with 1-4 R⁷, Si(alkyl)₃ or —(OCH₂CH₂)ₘ—Oalkyl;
R⁷ is —OP(═O)OH₂, C₁₋₆ alkyl.

33. The compound of embodiment 32, wherein R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, or arylene-NR⁵R⁶, 5-membered heteroaryl comprising two N atoms wherein the heteroaryl is substituted with alkyl, OH, halogen, haloC₁₋₃alkyl, OC₁₋₃alkyl or O-haloC₁₋₃alkyl;
each R^{A} is independently H, halo, unsubstituted alky, or alkylene-OH, or
two R^{A} together with the carbon they are connected to form a C₃₋₈ cycloalkylene or 3-10 membered heterocyclylene,
provided that at least one R^{A} is not H.

34. The compound of embodiment 32, wherein R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, or arylene-NR⁵R⁶, 5-membered heteroaryl comprising two N atoms wherein the heteroaryl is substituted with C₁₋₃alkyl;
each R^{A} is independently H, halo, unsubstituted alky, or alkylene-OH, provided that at least one R^{A} is not H.

35. The compound of any one of embodiments 32-34, wherein the compound is a compound of Formula (II)

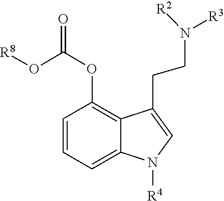

(II), or a pharmaceutically acceptable salt or deuterated form thereof,
   wherein:
R⁸ is —(CH₂CH₂O)ₘ-alkyl;
m is 2, 3 5 or 6;
R² and R³ are independently alkyl;
R⁴ is H or C(═O)Oalkyl; and

36. The compound of embodiment 35, wherein R⁸ is —(CH₂CH₂O)₂—CH₃ or —(CH₂CH₂O)₃—CH₃.

37. The compound of any one of embodiments 32-34, wherein the compound is a compound of Formula (III)

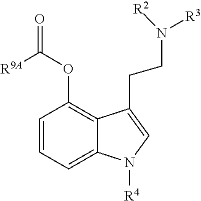

(III), or a pharmaceutically acceptable salt or deuterated form thereof,
   wherein:
R^{9A} is —C(R^{A})₂—O-alkyl, —C(R^{A})₂—O-cycloalkyl, cycloalkyl substituted with OC₁₋₆ alkyl, heterocyclyl substituted with C(═O)Oalkyl, 5-membered heteroaryl comprising two N atoms wherein the heteroaryl is substituted with C₁₋₃ alkyl, or arylene-NR⁵R⁶;
each R^{A} is independently H, halo, unsubstituted alky, or alkylene-OH, or
two R^{A} together with the carbon they are connected to form a C₃₋₈ cycloalkylene or 3-10 membered heterocyclylene,
provided that at least one R^{A} is not H;
R² and R³ are independently alkyl;
R⁴ is H or C(═O)Oalkyl;

38. The compound of embodiment 37, wherein
R^{9A} is —C(R^{A})₂—O-C₁₋₆ alkyl, —C(R^{A})₂—O-C₃₋₆ cycloalkyl, cyclopropyl substituted with OC₁₋₆ alkyl, heterocyclyl substituted with C(═O)OC₁₋₆alkyl, 5-membered heteroaryl comprising two N atoms wherein the heteroaryl is substituted with C₁₋₃ alkyl, or arylene-NR⁵R⁶; each R^{A} is independently H or unsubstituted alkyl, provided that at least one R^{A} is not H,
R⁵ and R⁶ are independently H, alkyl, or C(═O)alkyl;
R⁷ is OH, halo, alkyl, or haloalkyl.

39. The compound of any one of embodiments 32-34 or 37-38, wherein R^{9A} is

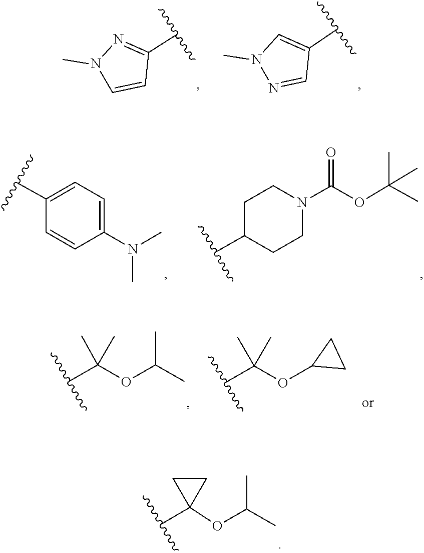

40. The compound of any one of embodiments 32-34, wherein the compound is a compound of Formula (IV)

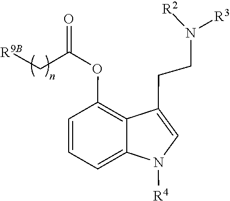

(IV), or a pharmaceutically acceptable salt or deuterated form thereof,
   wherein:
n is 1, 2, 3, 4, 5, or 6;
R^{9B} is branched alkylene-aryl wherein the aryl is substituted with 1-4 R⁷, Si(alkyl)₃ or —(OCH₂CH₂)ₘ—Oalkyl;
m is 2 or 3;
R² and R³ are independently alkyl;
R⁴ is H or C(═O)Oalkyl;
R⁷ is —OP(═O)OH₂ or C₁₋₆ alkyl;
provided that when R^{9B} is Si(alkyl)₃, then n is not 1.

41. The compound of embodiment 40, wherein n is 1, 2, or 3.

42. The compound of any one of embodiments 40-41, wherein R^{9B} is

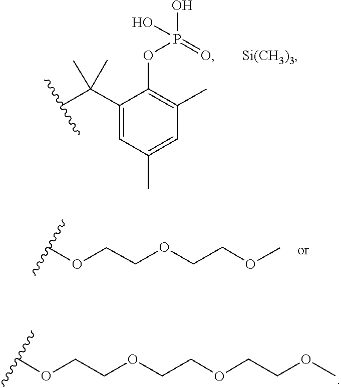

43. The compound of embodiment 40-42, wherein n is 1, 2 or 3 and R^{9B}

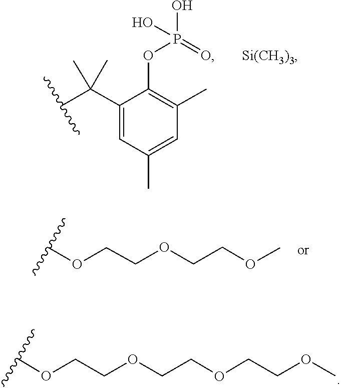

44. A compound of Formula (VII)

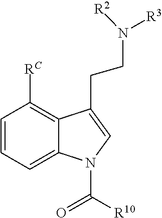

(VII), or a pharmaceutically acceptable salt or deuterated form thereof,
   wherein:
R² and R³ are independently alkyl;
R^{c} is OH or OC(═O)alkyl;
R¹⁰ is aryl;
R^{5A} and R^{6A} are independently alkyl or alkylene-OC(═O)alkyl.

45. The compound of embodiment 44, wherein R^{c} is OH, and R¹⁰ is phenyl.

46. The compound of any one of embodiments 34-45, wherein R² and R³ are independently C₁₋₆ alkyl.

47. The compound of any one of embodiments 34-46, wherein R² and R³ are —CH₃.

48. The compound of any one of embodiments 34-47, wherein R⁴ is H or C(═O)OC₁₋₆ alkyl.

49. The compound of any one of embodiments 34-48, wherein R⁴ is H or C(═O)OCH₃.

50. The compound of any one of embodiments 34-49, wherein R⁴ is H.

51. The compound of any one of embodiments 34-50, wherein R² and R³ are independently CH₃, and R⁴ is H.

52. A compound selected from Table 1, a pharmaceutically acceptable salt thereof, or a deuterated form thereof.

53. A pharmaceutical composition comprising a compound of embodiments 32-51, a pharmaceutically acceptable salt thereof, or a deuterated form thereof.

54. A method of treating a disease comprising administrating a pharmaceutical composition of embodiment 53.

55. A method of treating a disease comprising subcutaneously administrating a pharmaceutical composition of embodiment 53.

56. A method of treating a disease comprising administrating a pharmaceutical composition comprising a compound of Table 1, a pharmaceutically acceptable salt thereof, or a deuterated form thereof.

57. A method of treating a disease comprising subcutaneously administrating a pharmaceutical composition comprising a compound of Table 1, a pharmaceutically acceptable salt thereof, or a deuterated form thereof.

58. The method of any one of embodiments 55-57, wherein the disease is a 5-HT2A receptor associated disease or disorder.

59. The method of embodiment 58, wherein the neuropsychiatric disease is selected from anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, anxiety disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), and premenstrual syndrome (PMS).

### EXAMPLES

### Example 1. Synthesis of Compound 103: 1-(tert-butyl) 4-(3-(2-(dimethylamino)ethyl)-1H-indol-4-yl) piperidine-1,4-dicarboxylate, Formic Acid

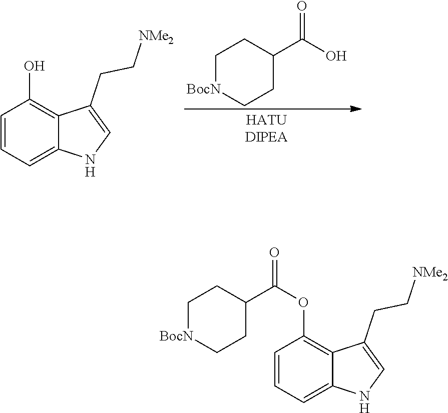 [SMILES: CN(C)CCc1c[nH]c2cccc(O)c12 ; CC(C)(C)OC(=O)N1CCC(C(=O)O)CC1 ; CN(C)CCc1c[nH]c2cccc(OC(=O)C3CCN(C(=O)OC(C)(C)C)CC3)c12]

A mixture of psilocin (201.0 mg, 1 Eq, 925 μmol), 1-(tert-butoxycarbonyl)piperidine-4-carboxylic acid (252.5 mg, 1 Eq, 925 μmol), HATU (507 mg, 1.4 Eq, 1.33 mmol) and DIPEA (282 mg, 377 μL, 2.4 Eq, 2.18 mmol) in dry DMF (2.5 mL) was stirred at r.t. for 16 h. The reaction mixture was diluted with sat. aq. NaHCO₃ (25 mL) and extracted with EtOAc (3×25 mL). The combined organic layers were washed sequentially with water (3×20 mL), brine (25 mL), dried (Na₂SO₄), filtered, and concentrated in vacuo. The crude product was loaded onto celite and purified by chromatography on RP Flash C18 (40 g cartridge, 5-50% (0.1% formic acid in MeCN)/(0.1% formic acid in water)) to afford the title compound (165 mg, 0.34 mmol, 37%) as a brown oil.

m/z 416.3 (M+H)⁺ (ES+)

¹H NMR (500 MHz, DMSO-d₆) δ 11.20-10.95 (m, 1H), 8.21 (s, 1H), 7.24 (d, J=8.1 Hz, 1H), 7.16 (d, J=2.3 Hz, 1H), 7.08-7.00 (m, 1H), 6.65 (d, J=7.6 Hz, 1H), 3.96 (d, J=13.2 Hz, 2H), 2.96-2.78 (m, 5H), 2.67 (dd, J=9.2, 6.5 Hz, 2H), 2.33 (s, 6H), 2.05 (dd, J=13.6, 3.7 Hz, 2H), 1.61 (qd, J=11.8, 4.2 Hz, 2H), 1.41 (s, 9H). 1× exchangeable H not observed.

### Example 2. Synthesis of Compound 104: 3-(2-(dimethylamino)ethyl)-1H-indol-4-yl 2-isopropoxy-2-methylpropanoate, Fumaric acid

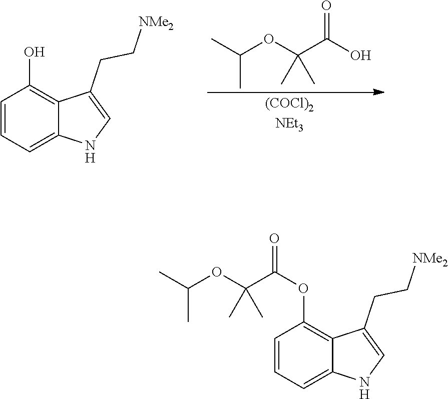

To a stirred solution of 2-isopropoxy-2-methylpropanoic acid (141 mg, 1 Eq, 963 μmol) in dry DCM (4 mL) under an atmosphere of nitrogen at r.t. was added oxalyl chloride (257 mg, 177 μL, 2.1 Eq, 2.02 mmol) and a drop of DMF. The reaction mixture was stirred at room temp for 2 h. The volatiles were removed in vacuo. The residue was dissolved in DCM (2 mL) and added to a solution of psilocin (200.7 mg, 1 Eq, 963 μmol) and triethylamine (351 mg, 483 μL, 3.6 Eq, 3.47 mmol) in DCM (2 mL) at 0° C. The reaction mixture was stirred at r.t. for 1 h. The reaction mixture was diluted with water (5 mL) and transferred into a separating funnel. The aqueous layer was extracted with DCM (3×5 mL). The combined organic layers were collected, dried (Na₂SO₄), filtered and concentrated in vacuo. The crude product was purified by chromatography on RP Flash C18 (24 g cartridge, 5-40% (0.1% formic acid in MeCN)/(0.1% formic acid in water)) to afford 3-(2-(dimethylamino)ethyl)-1H-indol-4-yl 2-isopropoxy-2-methylpropanoate, formic acid (139.2 mg, 367 μmol, 38%) as a brown oil. The material was dissolved in acetone (5 mL) and a solution of fumaric acid (43 mg, 0.38 Eq, 368 μmol) in acetone (7 mL) was added. The resulting solid was filtered, washed with acetone and dried in a vacuum desiccator for 24 h to afford the title compound (80.9 mg, 0.17 mmol, 18%) as a beige solid.

m/z 333.2 (M+H)⁺ (ES+)

¹H NMR (500 MHz, DMSO-d₆) δ 11.12 (s, 1H), 7.25 (dd, J=8.1, 0.8 Hz, 1H), 7.18 (d, J=2.3 Hz, 1H), 7.05 (m, 1H), 6.64 (d, J=7.6 Hz, 1H), 6.56 (s, 2H), 3.92 (hept, J=6.2 Hz, 1H), 2.90-2.83 (m, 2H), 2.77-2.70 (m, 2H), 2.34 (s, 6H), 1.57 (s, 6H), 1.16 (d, J=6.0 Hz, 6H). 2× exchangeable H not observed.

### Example 3. Synthesis of Compound 105: 3-(2-(dimethylamino)ethyl)-1H-indol-4-yl 4-(dimethylamino)benzoate, Formic Acid

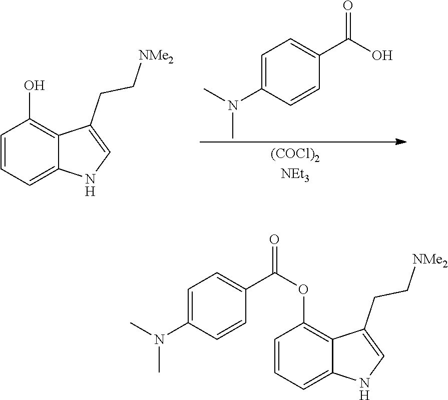 [SMILES: CN(C)CCc1c[nH]c2cccc(O)c12 ; CN(C)c1ccc(C(=O)O)cc1 ; CN(C)CCc1c[nH]c2cccc(OC(=O)c3ccc(N(C)C)cc3)c12]

To a stirred solution of 4-(Dimethylamino)benzoic acid (231 mg, 1.4 Eq, 1.40 mmol) in dry DCM (7 mL) under an atmosphere of nitrogen at r.t. was added oxalyl chloride (260 mg, 179 μL, 2.1 Eq, 2.05 mmol) and a drop of DMF. The reaction mixture was stirred at r.t. for 2 h. The volatiles were removed in vacuo. The residue was dissolved in DCM (2 mL) and added to a solution of psilocin (199.3 mg, 1 Eq, 976 μmol) and triethylamine (355 mg, 490 μL, 3.6 Eq, 3.51 mmol) in DCM (2 mL) at 0° C. The reaction mixture was stirred at r.t. for 1 h. The reaction mixture was diluted with water (5 mL) and transferred into a separating funnel. The aqueous layer was extracted with DCM (3×5 mL). The combined organic layers were collected, dried (Na₂SO₄), filtered and concentrated in vacuo. The crude product was purified by chromatography on RP Flash C18 (24 g cartridge, 5-50% (0.1% formic acid in MeCN)/(0.1% formic acid in water)) to afford 3-(2-(dimethylamino)ethyl)-1H-indol-4-yl 4-(dimethylamino)benzoate, Formic Acid (121.7 mg, 0.29 mmol, 30%) as a light brown solid. The material was dissolved in acetone (5 mL) and a solution of fumaric acid (36 mg, 0.3 Eq, 306 μmol) in acetone (5 mL) was added. The resulting solid was filtered, washed with acetone and dried in a vacuum desiccator overnight to afford the title compound (78.6 mg, 0.19 mmol, 20%) as an beige solid.

m/z 352.2 (M+H)⁺ (ES+)

¹H NMR (500 MHz, DMSO-d₆) δ 11.04 (s, 1H), 8.20 (s, 1H), 8.02-7.95 (m, 2H), 7.25 (dd, J=8.2, 0.8 Hz, 1H), 7.14 (d, J=2.3 Hz, 1H), 7.09-7.03 (m, 1H), 6.84-6.77 (m, 2H), 6.71 (dd, J=7.6, 0.8 Hz, 1H), 3.05 (s, 6H), 2.73 (dd, J=9.1, 6.6 Hz, 2H), 2.53-2.50 (m, 2H), 2.04 (s, 6H). 1× exchangeable H not observed.

### Example 4. Synthesis of Compound 108: 3-(2-(Dimethylamino)ethyl)-1H-indol-4-yl 1-methyl-1H-pyrazole-4-carboxylate, Formic Acid

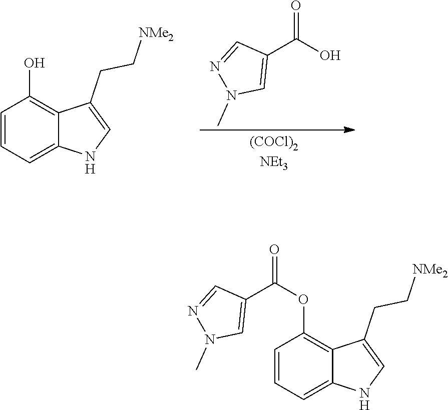

To a stirred solution of 1-methyl-1H-pyrazole-4-carboxylic acid (128 mg, 1.1 Eq, 1.01 mmol) in dry DCM (4.0 mL) under a N₂ atmosphere at rt was added oxalyl chloride (140 mg, 97.0 μL, 1.2 Eq, 1.10 mmol) and a drop of DMF. The reaction mixture was stirred at rt for 3 h. The volatiles were removed in vacuo. The residue was redissolved in DCM (2.0 mL) and added to a solution of psilocin (200 mg, 94% Wt, 1 Eq, 920 μmol) and Et₃N (466 mg, 641 μL, 5 Eq, 4.60 mmol) in DCM (2.0 mL) at 25° C. The resulting mixture was stirred at rt for 16 h. The reaction mixture was diluted with distilled water (25 mL) and extracted with DCM (3×25 mL). The combined organic layers were collected, dried (Na₂SO₄), filtered and concentrated in vacuo. The crude product was purified by chromatography on RP Flash C18 (24 g cartridge, 5-20% (0.1% formic acid in MeCN)/(0.1% formic acid in water)) to afford the title compound (149.9 mg, 0.38 mmol, 42%, 92% Purity) as a sticky orange glass/oil. In attempting to form the fumaric salt of the desired compound, the material was initially dissolved in acetone from which a turgid precipitate formed. After sonication of the suspension, the solid was recovered by filtration and dried in a desiccator at 50° C. for 16 h to afford the title compound (11.7 mg, 32.6 μmol, 3.6%, 100% Purity) as a white solid.

m/z 313.2 (M+H)⁺ (ES+)

¹H NMR (500 MHz, DMSO) δ 11.08 (s, 1H), 8.60 (s, 1H), 8.21 (s, 1H), 8.12-8.05 (m, 1H), 7.26 (dd, J=8.1, 0.8 Hz, 1H), 7.17 (d, J=2.3 Hz, 1H), 7.12-7.03 (m, 1H), 6.74 (dd, J=7.6, 0.8 Hz, 1H), 3.94 (s, 3H), 2.82-2.72 (m, 2H), 2.61-2.53 (m, 2H), 2.14 (s, 6H). 1H not observed (exchangeable proton of formic acid).

### Example 5. Synthesis of Compound 109: 3-(2-(Dimethylamino)ethyl)-1H-indol-4-yl 1-methyl-1H-pyrazole-3-carboxylate, Formic Acid

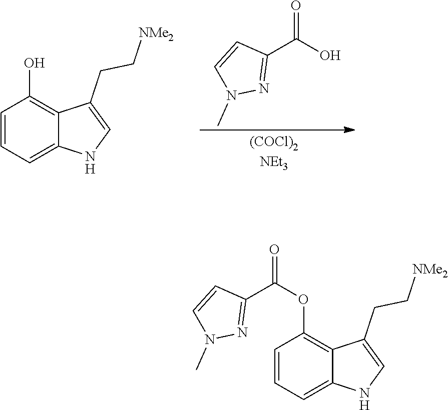

To a stirred solution of 1-methyl-1H-pyrazole-3-carboxylic acid (138.3 mg, 1.190 Eq, 1.097 mmol) in dry DCM (4.0 mL) under a N₂ atmosphere at rt was added oxalyl chloride (173 mg, 120 μL, 1.48 Eq, 1.37 mmol) and a drop of DMF. The reaction mixture was stirred at rt for 2 h. The volatiles were removed in vacuo. The residue was redissolved in DCM (2.0 mL) and added to a solution of psilocin (200.3 mg, 94% Wt, 1 Eq, 921.7 μmol) and Et₃N (466.3 mg, 642 μL, 5 Eq, 4.609 mmol) in DCM (2.0 mL) at 0° C. The resulting mixture was stirred at rt for 2 h. The reaction mixture was diluted with distilled water (10 mL), extracted with DCM (3×10 mL). The combined organic layers were collected, dried (Na₂SO₄), filtered and concentrated in vacuo. The crude product was purified by chromatography on RP Flash C18 (12 g cartridge, 5-40% (0.1% formic acid in MeCN)/(0.1% formic acid in water)) to afford impure product. The crude material was dissolved in DMSO (3 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130A, 5 μm, 30 mm×100 mm, flow rate 40 mL min-1 eluting with a 0.1% formic acid in water-MeCN gradient over 8.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 7.5% MeCN; 0.5-5.5 min, ramped from 7.5% MeCN to 35% MeCN; 5.5-5.6 min, ramped from 35% MeCN to 100% MeCN; 5.6-8.5 min, held at 100% MeCN. The clean fractions were evaporated in a Genevac, affording the title compound (181.3 mg, 0.49 mmol, 53%, 97% Purity) as a light brown oil.

m/z 313.2 (M+H)⁺ (ES+)

¹H NMR (500 MHz, DMSO) δ 11.08 (s, 1H), 8.17 (s, 1H), 7.94 (d, J=2.3 Hz, 1H), 7.27 (d, J=8.3 Hz, 1H), 7.16 (d, J=2.3 Hz, 1H), 7.07 (t, J=7.9 Hz, 1H), 6.98 (d, J=2.3 Hz, 1H), 6.75 (d, J=7.7 Hz, 1H), 3.99 (s, 3H), 2.77-2.71 (m, 2H), 2.07 (s, 6H). Two protons from CH₂ not observed. Signal overlaps with DMSO solvent peak. 1× exchangeable proton not observed.

### Example 6. Synthesis of Compound 110: 3-(2-(Dimethylamino)ethyl)-1H-indol-4-yl 3-(3,5-dimethyl-2-(phosphonooxy)phenyl)-3-methylbutanoate

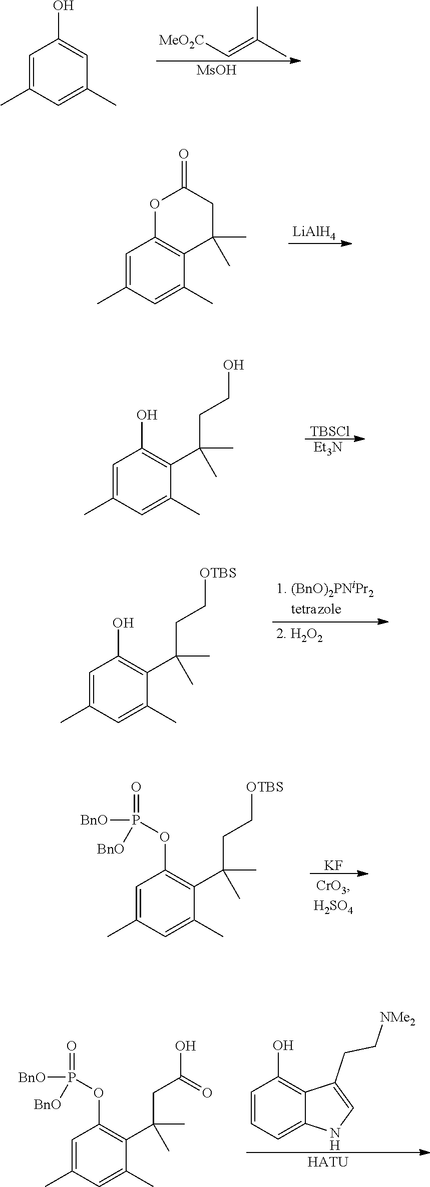

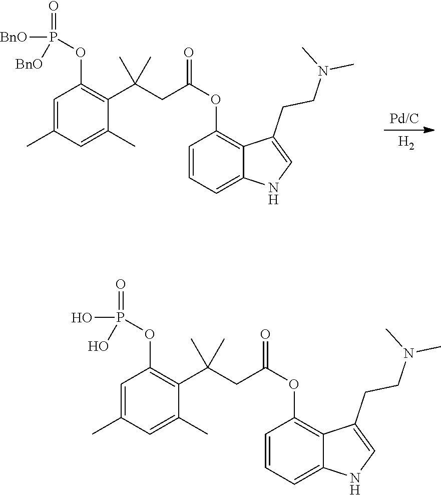 [SMILES: Cc1cc(C)c(C(C)(C)CC(=O)Oc2cccc3[nH]cc(CCN(C)C)c23)c(OP(=O)(OCc2ccccc2)OCc2ccccc2)c1 ; "H2" ; Cc1cc(C)c(C(C)(C)CC(=O)Oc2cccc3[nH]cc(CCN(C)C)c23)c(OP(=O)(O)O)c1]

### Step 1: 4,4,5,7-Tetramethylchroman-2-one

3,5-Dimethylphenol (4.449 g, 1 Eq, 36.42 mmol), methyl 3-methylbut-2-enoate (4.828 g, 1.162 Eq, 42.30 mmol) and methanesulfonic acid (6.66 g, 4.50 mL, 1.90 Eq, 69.3 mmol) were combined at rt then heated at 70° C. for 17 h. Upon cooling, the mixture was poured into water (700 mL) and extracted with EtOAc (2×200 mL). The combined organics were washed sequentially with sat. aq. NaHCO₃ (2×200 mL) and brine (100 mL), dried (Na₂SO₄) and concentrated in vacuo. The crude product was purified by chromatography on silica gel (120 g cartridge, 0-10% MeOH/DCM) (eluting 0%) to afford a mixture. The crude product was purified by chromatography on silica gel (80 g cartridge, 0-100% DCM/isohexanes) to afford the sub-title compound (6.402 g, 30 mmol, 82%, 95% Purity) as a yellow solid.

m/z 205.2 (M+H)⁺ (ES+)

¹H NMR (400 MHz, CDCl3) δ 6.66 (s, 2H), 2.51 (s, 2H), 2.38 (s, 3H), 2.19 (s, 3H), 1.36 (s, 6H).

Step 2: 2-(4-Hydroxy-2-methylbutan-2-yl)-3,5-dimethylphenol

A solution of the product from step 1 above (6.40 g, 95% Wt, 1 Eq, 29.8 mmol) in dry THF (50.0 mL) was added dropwise over 30 min to a −41° C. solution of LiAlH₄ (2.26 g, 24.8 mL, 2.40 M in THF, 2 Eq, 59.5 mmol). The reaction mixture was allowed to gradually warm to rt and left to stir for 16 h. The reaction mixture was cooled to 0° C. and quenched by adding sodium sulphate decahydrate (3 g) whilst maintaining the temperature. Water (5 mL) and 2M NaOH solution (10 mL) was then added to the reaction mixture. The salts were filtered off on a pad of celite and washed further with EtOAc (25 mL). The filtrate was concentrated in vacuo to afford a colourless oil. The oil was re-diluted with EtOAc (25 mL) and brine (25 mL). The organic layer was collected, and the aq. layer extracted with EtOAc (2×20 mL). The combined organic layers were dried (MgSO₄), filtered and concentrated in vacuo to afford the sub-title compound (4.87 g, 21 mmol, 71%, 90% Purity) as a white solid.

¹H NMR (500 MHz, DMSO) δ 8.94 (s, 1H), 6.43 (d, J=2.0 Hz, 1H), 6.30 (d, J=2.0 Hz, 1H), 4.09 (t, J=4.9 Hz, 1H), 3.24-3.17 (m, 2H), 2.36 (s, 3H), 2.08 (s, 3H), 2.06-2.01 (m, 2H), 1.44 (s, 6H).

Step 3: 2-(4-((tert-Butyldimethylsilyl)oxy)-2-methylbutan-2-yl)-3,5-dimethylphenol

To a solution of the product from step 2 above (4.87 g, 90% Wt, 1 Eq, 21.0 mmol) in dry DCM (60 mL) was added TBSCl (3.49 g, 1.1 Eq, 23.1 mmol). The resulting suspension was cooled to 0° C. before adding Et₃N (8.52 g, 11.7 mL, 4 Eq, 84.2 mmol) dropwise over 30 min. The reaction mixture was allowed to gradually warm to rt and left to stir for 16 h. The reaction was quenched with water (50 mL). The organic layer was collected and the aq. layer was washed with DCM (2×50 mL). The combined organic layers were washed with 10% citric acid solution (50 mL) and brine (50 mL), then dried (MgSO₄), filtered and concentrated in vacuo to afford an off-white crystalline solid. Iso-hexane (100 mL) was added and the resulting suspension was cooled to −20° C. for 1 h. The suspension was then filtered, washed with ice-cold iso-hexane, and dried in vacuo to afford the sub-title compound (4.88 g, 14 mmol, 68%, 94% Purity) as a white crystalline solid.

¹H NMR (500 MHz, CDCl3) δ 6.49 (d, J=2.0 Hz, 1H), 6.40 (d, J=2.0 Hz, 1H), 5.56 (s, 1H), 3.59 (t, J=6.9 Hz, 2H), 2.45 (s, 3H), 2.18 (s, 3H), 2.11 (t, J=6.9 Hz, 2H), 1.55 (s, 6H), 0.87 (s, 9H), 0.01 (s, 6H).

Step 4: Dibenzyl (2-(4-((tert-butyldimethylsilyl)oxy)-2-methylbutan-2-yl)-3,5-dimethylphenyl) phosphate

To a stirred solution of the product from step 3 above (499.3 mg, 94% Wt, 1 Eq, 1.455 mmol) in dry DCM (15.0 mL) under a nitrogen atmosphere at rt was added [bis(benzyloxy)phosphanyl]bis(propan-2-yl)amine (756 mg, 735 μL, 1.50 Eq, 2.19 mmol) and 2H-tetrazole in MeCN (0.16 g, 5.0 mL, 0.45 molar, 1.5 Eq, 2.3 mmol). The reaction mixture was stirred at rt overnight. The reaction mixture was cooled to 0° C. and H₂O₂ (833 mg, 760 μL, 30% Wt, 5.05 Eq, 7.35 mmol) was added. The resulting mixture was stirred at rt for 2 h.

The reaction mixture was diluted with distilled water (25 mL), extracted with DCM (3×25 mL). The combined organic layers were collected, washed with brine (50 mL), dried (Na₂SO₄), filtered and concentrated in vacuo. The crude product was purified by chromatography on silica gel (12 g cartridge, 0-50% EtOAc/isohexane) to afford the sub-title compound (1.0365 g, 1.4 mmol, 98%, 80% Purity) as a colourless oil.

¹H NMR (400 MHz, CDCl3) δ 7.34-7.28 (m, 10H), 7.08 (s, 1H), 6.70 (s, 1H), 5.10 (d, J=8.2 Hz, 4H), 3.46 (dd, J=8.0, 6.9 Hz, 2H), 2.48 (d, J=2.2 Hz, 3H), 2.16 (d, J=3.9 Hz, 3H), 2.08 (t, J=7.5 Hz, 2H), 1.51 (s, 6H), 0.82 (d, J=2.9 Hz, 9H), −0.07 (s, 6H).

Step 5: 3-(2-((Bis(benzyloxy)phosphoryl)oxy)-4,6-dimethylphenyl)-3-methylbutanoic acid

To a stirred solution of the product from step 4 above (1.0365 g, 80% Wt, 1 Eq, 1.4228 mmol) in acetone (10.0 mL) at 0° C. was added KF (137.1 mg, 55.26 μL, 1.658 Eq, 2.360 mmol) and Jones' reagent (2.60 g, 2.00 mL, 2.41 molar, 3.39 Eq, 4.82 mmol) (prepared by slow addition of H₂SO₄ (0.92 g, 0.50 mL, 6.6 Eq, 9.4 mmol) at 0° C. to a solution of chromium(VI) oxide (481.8 mg, 0.18 mL, 3.387 Eq, 4.818 mmol) in water (1.50 mL)). The reaction mixture was stirred at 0° C. for 2 h. The reaction mixture was quenched with the addition of IPA (0.5 mL) and stirred at rt for 20 min. The volatiles were removed in vacuo. The residue was redissolved in water (20 mL), extracted with EtOAc (3×25 mL). The combined organic layers was collected, dried (Na₂SO₄), filtered and concentrated in vacuo to afford the sub-title compound (833.1 mg, 1.2 mmol, 85%, 70% Purity) as a colourless oil.

m/z 483.8 (M+H)⁺ (ES+)

¹H NMR (400 MHz, CDCl3) δ 7.36-7.31 (m, 10H), 6.98 (s, 1H), 6.73 (s, 1H), 5.11 (dd, J=8.5, 3.4 Hz, 4H), 2.85 (s, 2H), 2.51 (s, 3H), 2.13 (s, 3H), 1.61 (s, 6H). 1× exchangeable OH proton not observed.

Step 6: 3-(2-(Dimethylamino)ethyl)-1H-indol-4-yl 3-(2-((bis(benzyloxy)phosphoryl)oxy)-3,5-dimethylphenyl)-3-methylbutanoate

To a stirred solution of the product from step 5 above (833.1 mg, 70% Wt, 1.027 Eq, 1.209 mmol) in dry DMF (6.0 mL) at rt was added HATU (552.4 mg, 1.235 Eq, 1.453 mmol) and DIPEA (299 mg, 400 μL, 1.97 Eq, 2.31 mmol). The mixture was stirred at rt for 20 min. Psilocin (240.3 mg, 1 Eq, 1.176 mmol) was added and resulting mixture was stirred at rt overnight. The reaction mixture was diluted with water (10 mL), extracted with EtOAc (3×25 mL). The combined organic layers were collected, dried (Na₂SO₄), filtered and concentrated in vacuo. The crude product was purified by chromatography on silica gel (12 g cartridge, 0-10% (0.7 M ammonia/MeOH)/DCM) to afford impure product. The impure product was further purified by chromatography on RP Flash C18 (12 g cartridge, 15-75% (0.1% formic acid in MeCN)/(0.1% formic acid in water)) to afford the sub-title compound (31.6 mg, 46 μmol, 3.9%, 97% Purity) as a beige solid.

m/z 669.3 (M+H)⁺ (ES+)

¹H NMR (400 MHz, MeOD) δ 7.33-7.22 (m, 10H), 7.21 (d, J=8.1 Hz, 1H), 7.15 (s, 1H), 7.00-6.92 (m, 2H), 6.81 (d, J=1.9 Hz, 1H), 6.25 (d, J=7.6 Hz, 1H), 5.12 (d, J=8.8 Hz, 4H), 3.27 (s, 2H), 3.22 (t, J=7.2 Hz, 2H), 2.97 (t, J=7.1 Hz, 2H), 2.71 (s, 6H), 2.56 (s, 3H), 2.14 (s, 3H), 1.71 (s, 6H). 1× exchangeable NH proton not observed.

Step 7: 3-(2-(Dimethylamino)ethyl)-1H-indol-4-yl 3-(3,5-dimethyl-2-(phosphonooxy)phenyl)-3-methylbutanoate

To a solution of the product from step 6 above (31.6 mg, 97% Wt, 1 Eq, 45.8 μmol) in MeOH was added Pd/C 87 L (10.3 mg, 5% Wt, 0.106 Eq, 4.84 μmol). The reaction mixture was stirred at rt with 2 bar of H₂ for 1 h. The mixture was filtered and the volatiles were removed in vacuo to afford the title compound (23.81 mg, 48 μmol, 100%, 98% Purity) as a beige solid.

m/z 489.2 (M+H)⁺ (ES+)

¹H NMR (500 MHz, MeOD) δ 7.24-7.17 (m, 2H), 7.11 (s, 1H), 7.04 (t, J=7.9 Hz, 1H), 6.65 (s, 1H), 6.57 (d, J=7.7 Hz, 1H), 3.50 (s, 2H), 3.30-3.28 (m, 2H), 3.07 (t, J=7.9 Hz, 2H), 2.83 (s, 6H), 2.55 (s, 3H), 2.19 (s, 3H), 1.74 (s, 6H). 3× exchangeable protons not observed.

### Example 7. Synthesis of Compound 111: 3-(2-(dimethylamino)ethyl)-1H-indol-4-yl 3-(trimethylsilyl)propanoate, 0.5Fumaric acid and 3-(2-(dimethylamino)ethyl)-1H-indol-4-yl 3-(trimethylsilyl)propanoate, Fumaric acid

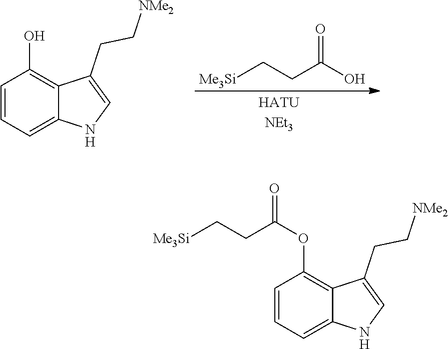 [SMILES: CN(C)CCc1c[nH]c2cccc(O)c12 ; C[Si](C)(C)CCC(=O)O ; CN(C)CCc1c[nH]c2cccc(OC(=O)CC[Si](C)(C)C)c12]

A mixture of psilocin (209.4 mg, 1.0 Eq, 1.03 mmol), 3-(trimethylsilyl)propanoic acid (180 mg, 1.2 Eq, 1.23 mmol), HATU (561 mg, 1.4 Eq, 1.48 mmol) and diisopropylethylamine (1.35 g, 1.8 mL, 10 Eq, 10.4 mmol) in dry DMF (2.5 mL) was stirred at r.t. for 18 h. The mixture was diluted with EtOAc (5 mL) and poured into water (5 mL). The phases were separated and the organic phase was washed sequentially with water:brine (1:1, 10 mL) and brine (10 mL). The organic phase was dried (Na₂SO₄), filtered, and concentrated in vacuo. The crude product was purified by chromatography on RP Flash C18 (12 g cartridge, 5-50% (0.1% formic acid in MeCN)/(0.1% formic acid in water)) (eluting ˜20%) to afford 3-(2-(dimethylamino)ethyl)-1H-indol-4-yl 3-(trimethylsilyl)propanoate, Formic Acid (188 mg, 497 μmol, 48%) as a thick brown oil. The material was dissolved in acetone (3 mL) and a solution of fumaric acid (60 mg, 1 Eq, 517 μmol) in acetone (5 mL) was added. The mixture was left at −20° C. for 18 h. The volume was reduced to −2 mL and the mixture was left at −20° C. for 64 h. The resulting solid was isolated by filtration and washed with MeCN (3×3 mL) to afford afford 3-(2-(dimethylamino)ethyl)-1H-indol-4-yl 3-(trimethylsilyl)propanoate, 0.5Fumaric acid (38.2 mg, 97.8 μmol, 10%) as a grey solid. The filtrate was left at −20° C. for 72 h. The filtrate was left at −20° C. for 72 h and a further solid was isolated by filtration and dried in vacuo to afford 3-(2-(dimethylamino)ethyl)-1H-indol-4-yl 3-(trimethylsilyl)propanoate, Fumaric acid (54.0 mg, 0.12 mmol, 24%, 99% Purity) as a grey solid.

### 3-(2-(dimethylamino)ethyl)-1H-indol-4-yl 3-(trimethylsilyl)propanoate, 0.5Fumaric Acid

m/z 333.2 (M+H)⁺ (ES+)

¹H NMR (500 MHz, DMSO-d₆) δ 12.73 (br, 1H), 11.03 (s, 1H), 7.22 (d, J=8.1 Hz, 1H), 7.14 (d, J=2.3 Hz, 1H), 7.03 (dd, J=7.9, 7.9 Hz, 1H), 6.65 (d, J=7.6 Hz, 1H), 6.54 (s, 1H), 2.82-2.74 (m, 2H), 2.70-2.61 (m, 2H), 2.56-2.51 (m, 2H), 2.27 (s, 6H), 0.97-0.85 (m, 2H), 0.05 (s, 9H).

### 3-(2-(dimethylamino)ethyl)-1H-indol-4-yl 3-(trimethylsilyl)propanoate, Fumaric Acid

m/z 333.2 (M+H)⁺ (ES+)

¹H NMR (500 MHz, DMSO-d₆) δ 12.96 (br, 2H), 11.06 (s, 1H), 7.23 (d, J=8.1 Hz, 1H), 7.16 (d, J=2.3 Hz, 1H), 7.04 (dd, J=7.9, 7.9 Hz, 1H), 6.66 (d, J=7.6 Hz, 1H), 6.56 (s, 2H), 2.88-2.76 (m, 2H), 2.71-2.60 (m, 4H), 2.35 (s, 6H), 0.96-0.85 (m, 2H), 0.05 (s, 9H).

### Example 8. Synthesis of Compound 112: 3-(2-(dimethylamino)ethyl)-1H-indol-4-yl 4-(trimethylsilyl)butanoate, Fumaric Acid

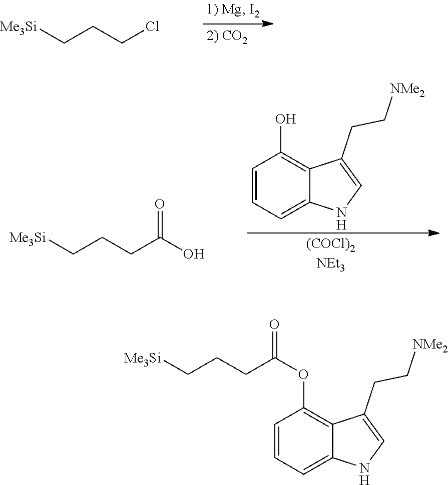 [SMILES: C[Si](C)(C)CCCCl ; C[Si](C)(C)CCCC(=O)O ; CN(C)CCc1c[nH]c2cccc(O)c12 ; CN(C)CCc1c[nH]c2cccc(OC(=O)CCC[Si](C)(C)C)c12]

Step 1: 4-(trimethylsilyl)butanoic Acid

In a 2-neck flask, charged with magnesium (242 mg, 1.5 Eq, 9.95 mmol), heated under vacuum (50° C., ˜15 min) and flushed with N₂, was added iodine (17 mg, 0.01 Eq, 66.3 μmol) and THF (1 mL). The mixture was heated at 70° C. and a solution of (3-chloropropyl)trimethylsilane (1.00 g, 1 Eq, 6.63 mmol) in THF (6 mL) was added dropwise. The reaction was left stirring at 70° C. for 2 h. After completion, the reaction was cooled to r.t. In a separate 2-neck flask with a bubbler were placed solid CO₂ pellets (˜3 g). The reaction mixture was then slowly added to the CO₂ pellets at −78° C. The mixture was gradually warmed to r.t. and was stirred for 24 h. The reaction mixture was quenched with aq. HCl 1M (10 mL) and transferred into a separating funnel. The aqueous layer was extracted with EtOAc (3×10 mL). The combined organic layers were collected, washed with brine (20 mL), dried (Na₂SO₄), filtered and concentrated in vacuo to afford the sub-title compound (808 mg, 4.8 mmol, 72%) as a light yellow oil.

¹H NMR (500 MHz, DMSO-d₆) δ 11.94 (s, 1H), 2.20 (t, J=7.3 Hz, 2H), 1.55-1.45 (m, 2H), 0.52-0.43 (m, 2H), −0.03 (s, 9H).

Step 2: 3-(2-(dimethylamino)ethyl)-1H-indol-4-yl 4-(trimethylsilyl)butanoate, Fumaric Acid

To a stirred solution of the product from step 1 above (155 mg, 1 Eq, 969 μmol) in dry DCM (4 mL) under an atmosphere of nitrogen at r.t. was added oxalyl chloride (258 mg, 178 μL, 2.1 Eq, 2.04 mmol) and a drop of DMF. The reaction mixture was stirred at r.t. for 2 h. Volatiles were removed in vacuo. The residue was dissolved in DCM (2 mL) and added to a solution of psilocin (202.0 mg, 1 Eq, 969 μmol) and triethylamine (353 mg, 486 μL, 3.6 Eq, 3.49 mmol) in DCM (2 mL) at 0° C. The reaction mixture was stirred at r.t. for 1 h. The reaction mixture was diluted with water (5 mL) and transferred into a separating funnel. The aqueous layer was extracted with DCM (3×5 mL). The combined organic layers were collected, dried (Na₂SO₄), filtered and concentrated in vacuo. The crude product was purified by chromatography on RP Flash C18 (24 g cartridge, 5-40% (0.1% formic acid in MeCN)/(0.1% formic acid in water)) to afford 3-(2-(dimethylamino)ethyl)-1H-indol-4-yl 4-(trimethylsilyl)butanoate, Formic Acid (187 mg, 437 μmol, 45%) as a yellow oil. The material was dissolved in acetone (5 mL) and a solution of fumaric acid (51 mg, 0.45 Eq, 437 μmol) in acetone (6 mL) was added. The resulting solid was filtered, washed with acetone (2×3 mL) and dried in a vacuum desiccator for 16 h to afford the title compound (114.8 mg, 0.24 mmol, 24%) as a light brown solid.

m/z 347.3 (M+H)⁺ (ES+)

¹H NMR (500 MHz, DMSO-d₆) δ 11.11 (s, 1H), 7.24 (dd, J=8.1, 0.9 Hz, 1H), 7.18 (d, J=2.4 Hz, 1H), 7.08-7.00 (m, 1H), 6.66 (dd, J=7.6, 0.9 Hz, 1H), 6.55 (s, 2H), 2.91-2.85 (m, 2H), 2.84-2.77 (m, 2H), 2.71 (t, J=7.3 Hz, 2H), 2.45 (s, 6H), 1.69 (dtd, J=9.1, 7.4, 4.7 Hz, 2H), 0.64-0.57 (m, 2H), 0.01 (s, 9H). 2× exchangeable H not observed.

### Example 9. Synthesis of compound 115: (3-(2-(dimethylamino)ethyl)-4-hydroxy-1H-indol-1-yl)(phenyl)methanone, Formic Acid

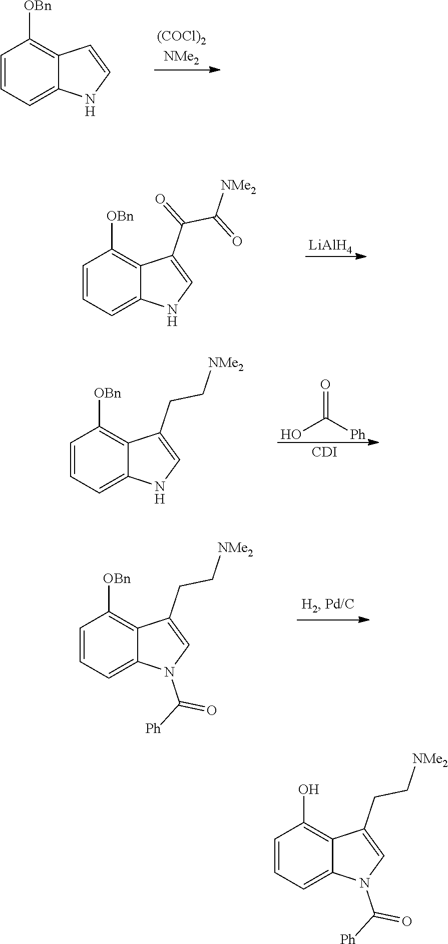

Step 1: 2-(4-(benzyloxy)-1H-indol-3-yl)-N,N-dimethyl-2-oxoacetamide (3573-105)

Oxalyl chloride (3.13 g, 2.2 mL, 1.1 Eq, 24.6 mmol) was added portion-wise to a stirred solution of 4-(benzyloxy)-1H-indole (5.00 g, 1 Eq, 22.4 mmol) in MTBE (100 mL) and stirred at r.t. for 90 min. The reaction mixture was then cooled to 0° C. and dimethylamine in THF (5.55 g, 61.6 mL, 2.0 M, 5.5 Eq, 123 mmol) added portion-wise over 5 min. The reaction mixture was left in the ice-bath which was allowed to warm slowly to r.t. over the course of another 90 min. The resultant suspension was filtered and the filtered solid partitioned between EtOAc (300 ml) and water (200 mL). The organic layer was separated, washed with sat. aq. NaHCO₃ (200 ml) and concentrated in vacuo to afford 2-(4-(benzyloxy)-1H-indol-3-yl)-N,N-dimethyl-2-oxoacetamide (4.76 g, 14.8 mmol, 66%) as a beige foam.

m/z 323.5 (M+H)⁺ (ES+)

¹H NMR (500 MHz, DMSO-d₆) δ 12.26 (s, 1H), 8.06 (s, 1H), 7.64-7.58 (m, 2H), 7.37 (dd, J=8.3, 6.9 Hz, 2H), 7.28 (t, J=7.3 Hz, 1H), 7.14-7.06 (m, 2H), 6.69 (dd, J=7.2, 1.5 Hz, 1H), 5.26 (s, 2H), 2.90 (d, J=16.8 Hz, 6H).

Step 2: 2-(4-(benzyloxy)-1H-indol-3-yl)-N,N-dimethylethan-1-amine

Lithium Aluminium hydride in THF (1.96 g, 21.5 mL, 2.4 molar, 3.5 Eq, 51.7 mmol) was added portion-wise to stirred suspension of the product from step 1 above (4.76 g, 1 Eq, 14.8 mmol) in 2-MeTHF (100 mL) at 0° C. and under nitrogen. The mixture was left to stir as the ice-bath warmed to r.t. for 30 min before being stirred at 80° C. for 4 h. The reaction mixture was allowed to cool to r.t. before sodium sulfate decahydrate was added portionwise until the resultant effervescence subsided. The reaction mixture was filtered, the solid plug washed with MeOH (30 mL) and the clear filtrate collected allowed to stand at r.t. overnight. The resulting dark green solution was concentrated under reduced pressure to afford a dark green oil which solidified on standing. The solid was partitioned between EtOAc (200 mL) and water (100 mL).

The organic layer was separated and concentrated under vacuum to afford the sub-title compound (4.16 g, 13 mmol, 90%) as a brown solid which solidified on standing.

m/z 295.2 (M+H)⁺ (ES+)

¹H NMR (500 MHz, DMSO-d₆) δ 10.75 (s, 1H), 7.56-7.50 (m, 2H), 7.39 (dd, J=8.3, 6.8 Hz, 2H), 7.36-7.29 (m, 1H), 6.97 (d, J=2.3 Hz, 1H), 6.96-6.89 (m, 2H), 6.53 (dd, J=6.0, 2.5 Hz, 1H), 5.16 (s, 2H), 2.94-2.87 (m, 2H), 2.50-2.42 (m, 2H), 2.05 (s, 6H).

Step 3: (4-(benzyloxy)-3-(2-(dimethylamino)ethyl)-1H-indol-1-yl)(phenyl)methanone

CDI (84 mg, 1.1 Eq, 520 μmol) was added to solution of benzoic acid (58 mg, 45 μL, 1 Eq, 473 μmol) in DCM (5 mL) and stirred at r.t. overnight. Separately, potassium tert-butoxide (58 mg, 1.1 Eq, 520 μmol) was added to a solution of the product of step 2 above (148 mg, 1 Eq, 473 μmol) in THF (5 mL) and stirred at r.t. overnight. The DCM solution was added to 4 mL to the THF solution and left to stir at r.t. for 21 h. The bulk solvent was removed from the reaction mixture and the residue partitioned between EtOAc (10 ml) and sat. aq. NaHCO₃ (10 mL). The organic layer was separated and adsorbed on to silica-gel. The crude product was purified by chromatography on silica gel (12 g cartridge, 0-10% (0.7 M ammonia/MeOH)/DCM) to afford the sub-title compound (63 mg, 158 μmol, 39%) as a pale yellow gum.

m/z 399.2 (M+H)⁺ (ES+)

¹H NMR (500 MHz, DMSO-d₆) δ 7.87 (d, J=8.3 Hz, 1H), 7.75-7.71 (m, 2H), 7.70-7.65 (m, 1H), 7.63-7.58 (m, 2H), 7.55-7.51 (m, 2H), 7.42-7.38 (m, 2H), 7.36-7.32 (m, 1H), 7.31-7.27 (m, 1H), 7.09 (s, 1H), 6.97 (d, J=8.1 Hz, 1H), 5.25 (s, 2H), 2.87 (t, J=7.8 Hz, 2H), 2.45 (t, J=7.7 Hz, 2H), 2.02 (s, 6H).

Step 4: (3-(2-(dimethylamino)ethyl)-4-hydroxy-1H-indol-1-yl)(phenyl)methanone, Formic Acid

The product from step 3 above (63 mg, 1 Eq, 150 μmol) in methanol (4.81 mg, 1 Eq, 150 μmol) was passed through a 10% Pd/C CatCart (H-cube, recirculation) at 1 mL/min, 60° C. and at 1 bar or 90 min. The reaction mixture was then left to stand at r.t. for 2 days. The bulk solvent was removed under vacuum to afford the crude product as brown gum. The crude material was dissolved in DMSO (1.52 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 A, 5 μm, 30 mm×100 mm, flow rate 40 mLmin⁻¹ eluting with a 0.1% formic acid in water-MeCN gradient over 12.5 min using UV across all wavelengths with PDA as well as a QDA and ELS detector. At column dilution pump gives 2 mLmin⁻¹ MeCN over the entire method, which is included in the following MeCN percentages. Gradient information: 0.0-0.5 min, 5% MeCN; 0.5-10.5 min, ramped from 5% MeCN to 35% MeCN; 10.5-10.6 min, ramped from 35% MeCN to 100% MeCN; 10.6-12.5 min, held at 100% MeCN. The clean fractions were evaporated in a Genevac to afford the title compound (10.0 mg, 28.2 μmol, 19%) as a colourless foam.

m/z 309.2 (M+H)⁺ (ES+)

¹H NMR (500 MHz, DMSO-d₆) δ 7.76-7.70 (m, 3H), 7.68-7.64 (m, 1H), 7.63-7.56 (m, 2H), 7.14-7.11 (m, 1H), 7.03 (s, 1H), 6.67 (dd, J=7.9, 0.9 Hz, 1H), 2.88 (t, J=6.9 Hz, 2H), 2.60 (t, J=6.9 Hz, 2H), 2.23 (s, 6H). (2× exchangeable H not observed).

### Example 10. Psilocin Release of Compounds

### Human Hepatocyte Clearance

The metabolic stability of compounds was assessed by monitoring the disappearance of parent compounds for up to 1 hour at 37° C. in cryopreserved hepatocytes, using established controls (Diltazem and Naloxone), to confirm suitability of the assay. Psilocin was also run as an additional control to monitor psilocin formation in a semi-quantitative manner.

Samples were analysed by UHPLC-MS/MS—Waters™ Acquity UPLC system, Waters™ Xevo TQ-XS on a Waters™ Acquity UPLC® HSS T3 column (1.8 μm, 2.1 mm×30 mm), mobile phases were water+0.1% formic acid and methanol+0.1% formic acid.

The elimination rate constant, half-life (t1/2) and intrinsic clearance (CLᵢₙₜ, μL/min/10⁶ cells) were determined using Ln(MS response) vs time plots. In addition, the appearance of psilocin from test compounds was monitored and assessed (as a percentage) against control psilocin peaks (at Time 0) to provide a semi-quantitative measure of psilocin release. The data are included in Table 4.

### Human Plasma Stability

The plasma stability of compounds was assessed by monitoring the disappearance of parent compounds for up to 2 hours at 37° C. in plasma in duplicate, using positive (Propantheline), negative (Pepstatin) and solvent controls (DMSO) to confirm suitability of the assay. Psilocin was also run as a control to monitor psilocin formation in a semi-quantitative manner.

Samples were analysed by UHPLC-MS/MS—Sciex™ MS500 Triple Quad QTRAP UHPLC system with a HESI-JI electrospray source on a Waters™ Acquity UPLC® HSS T3 column (1.8 pim, 2.1 mm×50 mm), mobile were phases water+0.1% formic acid and methanol+0.1% formic acid.

The elimination rate constant and half-life (t1/2) were determined using Ln(MS response) vs time plots. In addition, the appearance of psilocin from test compounds was monitored and assessed (as a percentage) against control psilocin peaks (at Time 0) to provide a semi-quantitative measure of psilocin release. The psilocin release data are included in table 4.

TABLE 4
psilocin release of the compounds

| | Human Plasma | Human | |
|---|---|---|---|
| Compound | Maximum % | Hepatocytes % | Psilocin Release |
| No. | Psilocin | Psilocin at 5 mins^{[1]} | Efficiency (%) ^{[2]} |
| 103 | 67 | NA | 67 |
| 104 | 100 | NA | 100 |
| 105 | 100 | 108 | 100 |
| 108 | 100 | NA | 100 |
| 109 | 100 | NA | 100 |
| 110 | 12 | 0.93 | 12 |
| 111 | 93 | NA | 93 |
| 115 | 98 | 32 | 98 |
| 141 | 100 | NA | 100 |
| 142 | 26 | NA | 26 |
| 143 | 18 | 19.8 | 18 |
| 144 | 0 | 35 | 0 |
| 145 | 2 | 25.5 | 2 |
| 146 | 100 | 238 | 100 |
| 147 | 60 | 70.2 | 60 |
| 148 | 100 | NA | 100 |
| 149 | 100 | 169 | 100 |
| 150 | 48 | NA | 48 |
| 151 | 11 | NA | 11 |
| 152 | 100 | NA | 100 |
| 153 | 59 | 70.5 | 59 |
| 154 | 100 | 128.2 | 100 |
| 155 | 100 | NA | 100 |
| 156 | 75 | NA | 75 |
| 157 | 100 | NA | 100 |
| 158 | 100 | NA | 100 |
| 159 | 100 | NA | 100 |
| 160 | 73 | NA | 73 |
| 161 | 100 | 116.8 | 100 |
| 162 | 25.5 | 47.65 | 25.5 |
| 163 | 8 | 7.3 | 8 |
| 164 | 100 | 89 | 100 |
| 165 | 24 | 8.82 | 24 |
| 166 | 4 | 12 | 4 |
| 168 | 100 | NA | 100 |
| 169 | 82 | 60.7 | 82 |
| 170 | 100 | 109 | 100 |
| 171 | 81 | NA | 81 |
| 172 | 100 | 81.5 | 100 |
| 173 | 100 | NA | 100 |
| 174 | 100 | NA | 100 |
| 175 | 91 | 160 | 91 |
| 176 | 100 | 100.3 | 100 |
| 177 | 5 | NA | 5 |
| 178 | 100 | 158 | 100 |
| 180 | 85 | NA | 85 |
| 182 | 2 | 10.2 | 2 |

^{[1]}“NA” means the data for this compound is not tested or not available.

^{[2]}Psilocin Release Efficiency (%) is calculated by the following equation:

Psilocin release efficiency (%)=_{theoretical maximum human plasma psilocin release}/^{actual human plasma maximum psilocin release}×100%

1. A compound of Formula (I) 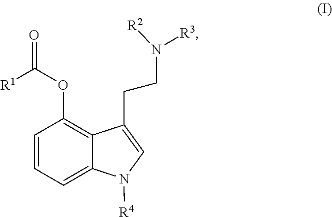
or pharmaceutically acceptable salt or deuterated form thereof,
wherein:
R² and R³ are independently alkyl;
R⁴ is H or C(═O)Oalkyl; and
R¹ is defined according to i, ii, iii, or iv:
i. R¹ is —OR⁸, wherein R⁸ is alkynyl substituted with NR⁵R⁶, or —(CH₂CH₂O)ₘ-alkyl, wherein m is 1, 2, 3, 5 or 6; and
R⁵ and R⁶ are independently H, alkyl, C(═O)alkyl, alkylene-aryl, or S(O)₂alkyl;
ii. R¹ is —R^{9A}, wherein R^{9A} is —C(R^{A})₂NR⁵R⁶, —C(R^{A})₂—O-alkyl, —C(R^{A})₂—O-cycloalkyl, cycloalkyl substituted with —Oalkyl, heterocyclyl substituted with —C(═O)Oalkyl, alkenylene-aryl wherein the aryl is substituted with 1-4 R^{7A}, alkenylene-heteroaryl wherein the heteroaryl is substituted with 0-4 R^{7A}, aryl substituted with NR⁵R⁶, or 5-membered heteroaryl substituted with 1-4 groups selected from alkyl, OH, halogen, haloalkyl, Oalkyl or O-haloalkyl;
each R^{A} is independently H, halo, unsubstituted alkyl, or alkylene-OH, or
two R^{A} together with the carbon they are connected to form a cycloalkylene or heterocyclylene,
provided that at least one R^{A} is not H;
R⁵ and R⁶ are independently H, alkyl, or 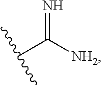 [SMILES: *C(=N)N]
or C(═O)alkyl wherein the alkyl is optionally substituted with NH₂, NH(alkyl), N(alkyl)₂, C(═O)aryl, C(═O)Oalkyl, S(O)₂alkyl, or alkylene-heterocyclyl; and
R^{7A} is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl;
provided that R^{9A} is not 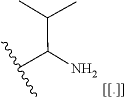 [SMILES: *C(N)C(C)C]
iii. R¹ is —(CH₂)ₙ—R^{9B}, wherein
n is 1, 2, 3, 4, 5, or 6;
R^{9B} is —O-alkylene-OH, —C(═O)-alkylene-NR⁵R⁶, alkylene-aryl wherein the aryl is substituted with 1-4 R^{7B}, Si(alkyl)₃ or —(OCH₂CH₂)ₘ—Oalkyl, m is 2 or 3, provided that when R^{9B} is Si(alkyl)₃ or 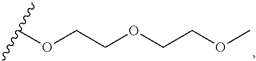 [SMILES: *OCCOCCOC]
then n is not 1;
R⁵ and R⁶ are independently H, alkyl, C(═O)alkyl, C(═O)aryl, C(═O)Oalkyl, S(O)₂alkyl, alkylene-heterocyclyl; and
R^{7B} is —OP(═O)OH₂, or C₁₋₆ alkyl;
iv. R¹ is —N(R^{5A})(R^{6A}), wherein
R^{5A} and R^{6A} are independently H, C₁₋₆ alkyl optionally substituted with —COOH, —OC(═O)alkyl or —O-alkylene-OH,
provided that when one of R^{5A} and R^{6A} is H, the other cannot be H, CH₂CH(CH₃)₂, or (CH₂)₅CH₃, and
when one of R^{5A} and R^{6A} is CH₃, the other cannot be H, CH₃, CH₂CH₃, CH₂CH(CH₃)₂,

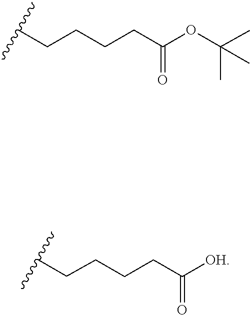 [SMILES: *CCCCC(=O)OC(C)(C)C ; *CCCCC(=O)O]

2. The compound of claim 1, wherein the compound is a compound of Formula (II) 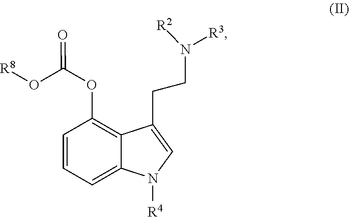
or a pharmaceutically acceptable salt or deuterated form thereof,
wherein:
R⁸ is alkynylene-NR⁵R⁶ or —(CH₂CH₂O)ₘ-alkyl;
m is 1, 2, 3, 5 or 6;
R² and R³ are independently alkyl;
R⁴ is H or C(═O)Oalkyl; and
R⁵ and R⁶ are independently H, alkyl, C(═O)H, C(═O)alkyl, alkylene-heteroaryl, alkylene-aryl, alkylene-carbocyclyl, alkylene-heterocyclyl, S(═O)alkyl, or S(═O)₂alkyl.

3. The compound of claim 2, wherein R⁸ is:
C₁₋₆ alkynyl substituted with N(C₁₋₆ alkyl)₂, or
—(CH₂CH₂O)ₘ—CH₃, wherein m is 1, 2 or 3.

4. The compound of claim 2, wherein R⁸ is 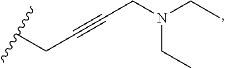 [SMILES: *CC#CCN(CC)CC]
—(CH₂CH₂O)—CH₃, —(CH₂CH₂O)₂—CH₃ or —(CH₂CH₂O)₃—CH₃.

5. The compound of claim 1, wherein the compound is a compound of Formula (III) 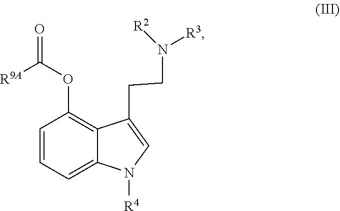
or a pharmaceutically acceptable salt or deuterated form thereof,
wherein:
R^{9A} is —C(R^{A})₂NR⁵R⁶, —C(R^{A})₂—O-alkyl, —C(R^{A})₂—O-cycloalkyl, cycloalkyl substituted with Oalkyl, heterocyclyl substituted with C(═O)Oalkyl, alkenylene-aryl wherein the aryl is substituted with 1-4 R^{7A}, alkenylene-heteroaryl wherein the heteroaryl is substituted with 0-4 R^{7A}, aryl substituted with NR⁵R⁶ or 5-membered heteroaryl substituted with 1-4 groups selected from alkyl, OH, halogen, haloalkyl, Oalkyl or O-haloalkyl;
each R^{A} is independently H, halo, unsubstituted alkyl, or alkylene-OH, or
two R^{A} together with the carbon they are connected to form a cycloalkylene or heterocyclylene,
provided that at least one R^{A} is not H;
R² and R³ are independently alkyl;
R⁴ is H or C(═O)Oalkyl;
R⁵ and R⁶ are independently H, alkyl, 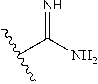 [SMILES: *C(=N)N]
or C(═O)alkyl wherein the alkyl is optionally substituted with NH₂, NH(alkyl) or N(alkyl)₂; and
R^{7A} is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl,
provided that R^{9A} is not

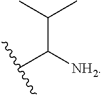 [SMILES: *C(N)C(C)C]

6. The compound of claim 5, wherein
R^{9A} is selected from —C(R^{A})₂NR⁵R⁶, —C(R^{A})₂—O-C₁₋₆ alkyl, —C(R^{A})₂—O-C₃₋₆cycloalkyl, cyclopropyl substituted with OC₁₋₆ alkyl, heterocyclyl substituted with C(═O)OC₁₋₆alkyl, C₁₋₆ alkenylene-aryl wherein the aryl is substituted with 1-4 R^{7A}, C₁-6 alkenylene-heteroaryl, arylene-NR⁵R⁶, or 5-membered heteroaryl comprising two N atoms wherein the heteroaryl is substituted with C₁₋₃ alkyl;
each R^{A} is independently H or unsubstituted alkyl, provided that at least one R^{A} is not H,
R² and R³ are independently alkyl;
R⁴ is H or C(═O)Oalkyl;
R⁵ and R⁶ are independently H, alkyl, 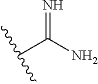 [SMILES: *C(=N)N]
or C(═O)alkyl wherein the alkyl is optionally substituted with NH₂, NH(alkyl) or N(alkyl)₂; and
R^{7A} is OH, halo, alkyl, haloalkyl, O-alkyl, OC(═O)alky, Si(alkyl)₃, aryl, NH(C═O)alkyl, NH₂, NH(alkyl), N(alkyl)₂, or heterocyclyl.

7. The compound of claim 5, wherein R^{9A} is

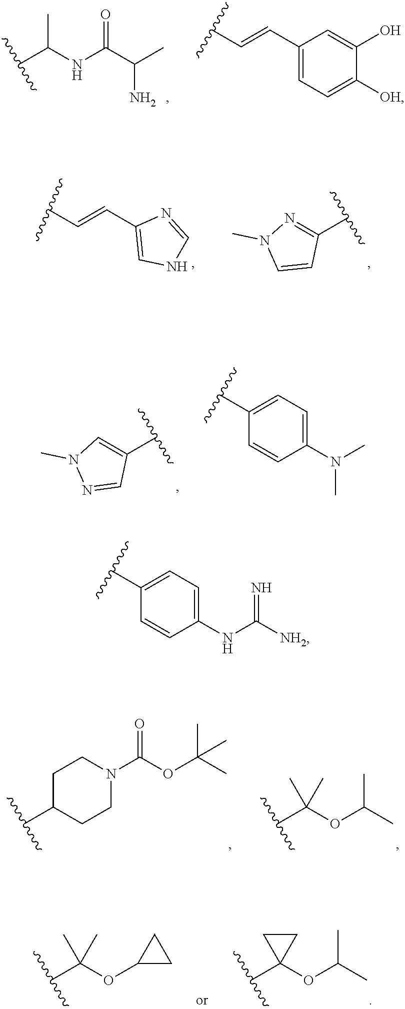

8. The compound of claim 1, wherein the compound is a compound of Formula (IV) 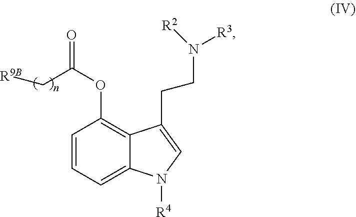
or a pharmaceutically acceptable salt or deuterated form thereof,
wherein:
n is 1, 2, 3, 4, 5, or 6;
R^{9B} is —O-alkylene-OH, —C(═O)-alkylene-NR⁵R⁶, alkylene-aryl wherein the aryl is substituted with 1-4 R^{7B}, Si(alkyl)₃ or —(OCH₂CH₂)ₘ—Oalkyl;
m is 2 or 3;
R² and R³ are independently alkyl;
R⁴ is H or C(═O)Oalkyl;
R⁵ and R⁶ are independently H, alkyl, C(═O)alkyl, C(═O)aryl, C(═O)Oalkyl, S(O)₂alkyl or alkylene-heterocyclyl; and
R^{7B} is —OP(═O)OH₂, or C₁₋₆ alkyl,
provided that when R^{9B} is Si(alkyl)₃ or 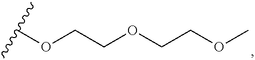 [SMILES: *OCCOCCOC]
then n is not 1.

9. The compound of claim 8, wherein
n is 1, 2 or 3;
R^{9B} is —O-C₁₋₆ alkylene-OH or —C(═O)-C₁₋₆ alkylene-NR⁵R⁶, C₁₋₆ alkylene-aryl wherein the aryl is substituted with 1-4 R^{7B}, Si(C₁-6 alkyl)₃, —(OCH₂CH₂)ₘ—Oalkyl;
m is 2 or 3;
R² and R³ are independently alkyl;
R⁴ is H or C(═O)OC₁₋₆ alkyl; and
R⁵ and R⁶ are independently H or alkyl.

10. The compound of claim 8, wherein R^{9B} is

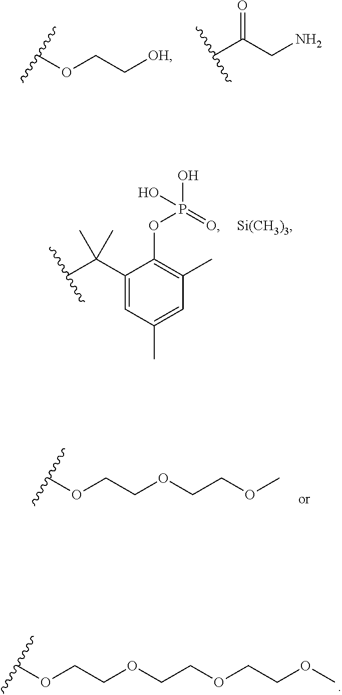

11. The compound of claim 8, wherein n is 1, 2 or 3, and R^{9B} is

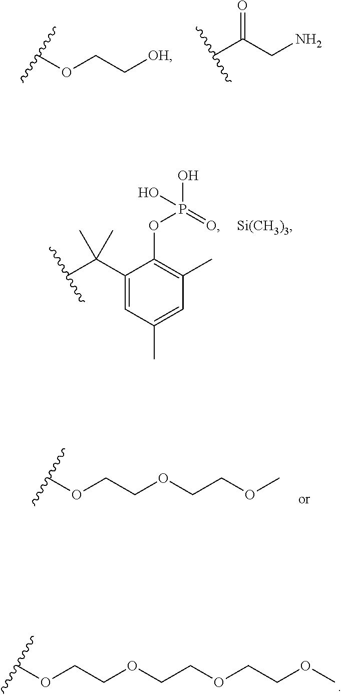

12. The compound of claim 1, wherein the compound is a compound of Formula (V) 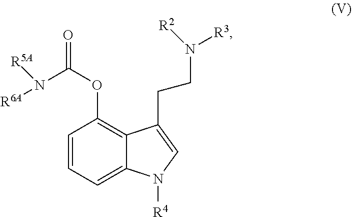
or a pharmaceutically acceptable salt or deuterated form thereof,
wherein:
R² and R³ are independently alkyl;
R⁴ is H or C(═O)Oalkyl;
R^{5A} and R^{6A} are independently H, C₁₋₆ alkyl optionally substituted with —COOH, —OC(═O)alkyl or —O-alkylene-OH,
provided that both R^{5A} and R^{6A} are not H, and when one of R^{5A} and R^{6A} is H, the other cannot be H, —(CH₂)₅CH₃ or CH₂CH(CH₃)₂, and
further provided that when one of R^{5A} and R^{6A} is CH₃, the other cannot be H, CH₃ CH₂CH₃, CH₂CH(CH₃)₂,

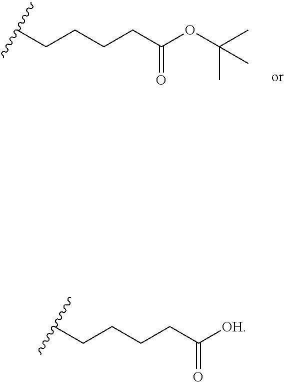 [SMILES: *CCCCC(=O)OC(C)(C)C ; *CCCCC(=O)O]

13. The compound of claim 12, wherein R^{5A} and R^{6A} are independently H, CH₃,

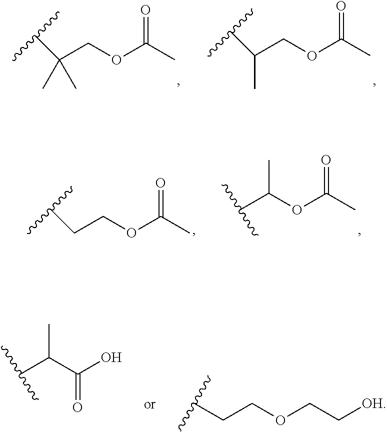

14. A compound of Formula (VI) 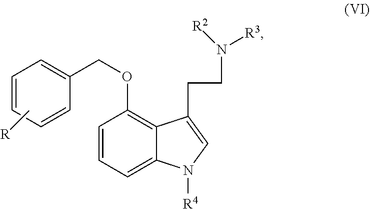
or a pharmaceutically acceptable salt or deuterated thereof,
wherein:
R is NR⁵R⁶;
R² and R³ are independently alkyl;
R⁴ is H or C(═O)Oalkyl; and
R⁵ and R⁶ are independently H, alkyl, haloalkyl, or C(═O)haloalkyl.

15. The compound of claim 14, wherein R⁵ is H and R⁶ is C(═O)CF₃.

16. A compound of Formula (VII) 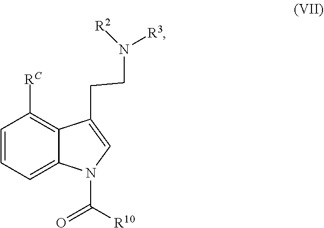
or a pharmaceutically acceptable salt or deuterated form thereof,
wherein:
R² and R³ are independently alkyl;
R^{c} is OH or OC(═O)alkyl;
R¹⁰ is NR^{5A}R^{6A} O-alkylene-OC(═O)alkyl, O-alkylene-Si(alkyl)₂, heterocycle, or aryl; and
R^{5A} and R^{6A} are independently alkyl or alkylene-OC(═O)alkyl.

17. The compound of claim 16, wherein
R^{c} is OH or OC(═O)C₁₋₆ alkyl;
R¹⁰ is NR^{5A}R^{6A} 0-C₁-6 alkylene-OC(═O)C₁₋₆ alkyl, —O-C₁₋₆ alkylene-Si(C₁₋₆ alkyl)₂, 3-8 membered heterocycle or aryl; and
R^{5A} and R^{6A} are independently C₁₋₆ alkyl or C₁₋₆ alkylene-OC(═O)C₁₋₆ alkyl.

18. The compound of claim 16, wherein
R^{c} is OH or OC(═O)CH₃, and
R¹⁰ is

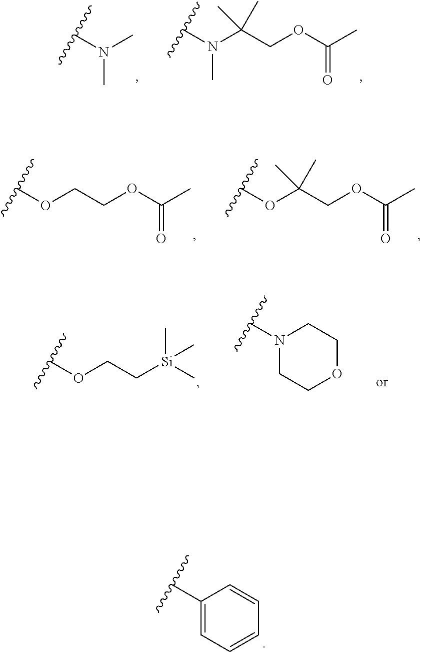

19. The compound of claim 1, wherein R² and R³ are independently C₁₋₆ alkyl.

20. The compound claim 1, wherein R² and R³ are —CH₃.

21. The compound claim 1, wherein R⁴ is H or C(═O)OC₁₋₆ alkyl.

22. The compound of claim 1, wherein R⁴ is H or C(═O)OCH₃.

23. The compound of claim 1, wherein R⁴ is H.

24. The compound of claim 1, wherein R² and R³ are CH₃, and R⁴ is H.

25-28. (canceled)

29. A method of treating a disease comprising subcutaneously administrating a pharmaceutical composition comprising a compound selected from: 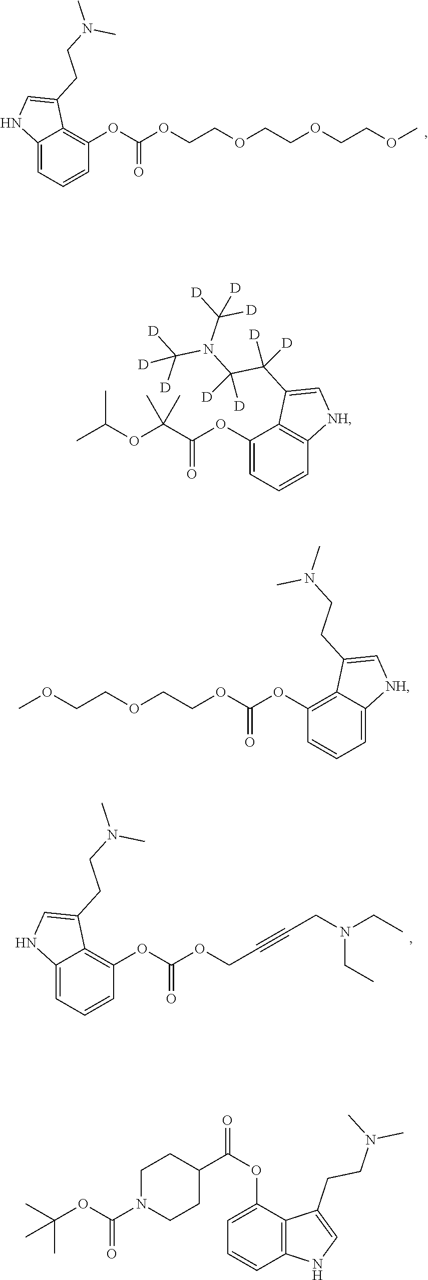 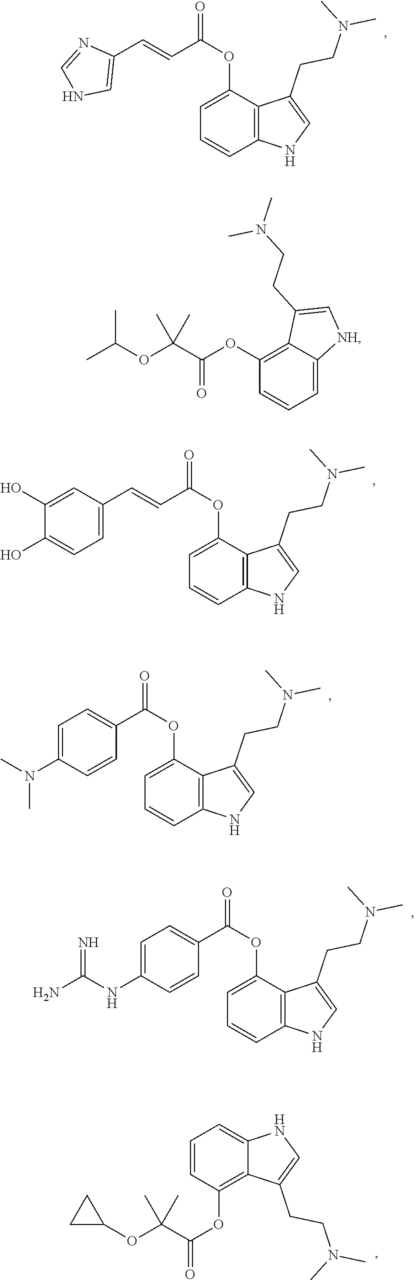 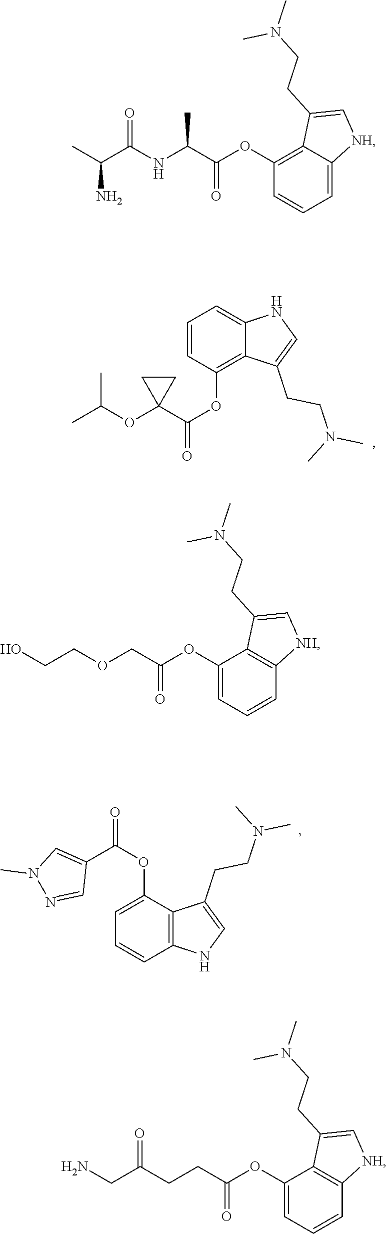 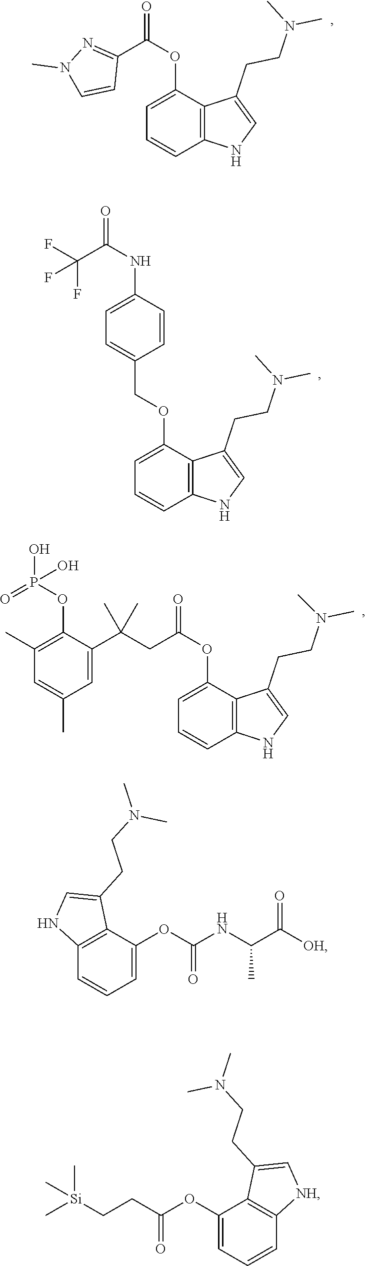 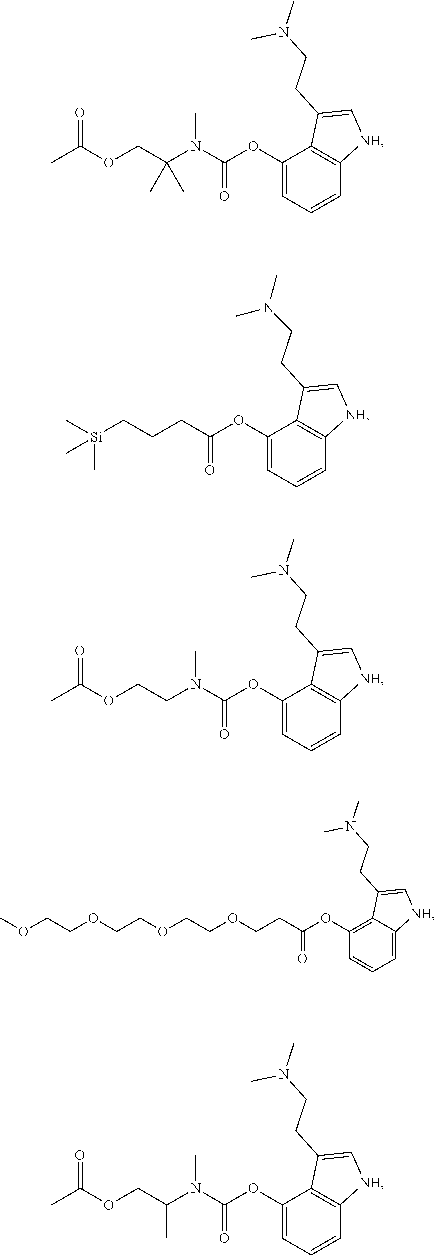 [SMILES: CC(=O)OCC(C)(C)N(C)C(=O)Oc1cccc2[nH]cc(CCN(C)C)c12 ; CN(C)CCc1c[nH]c2cccc(OC(=O)CCC[Si](C)(C)C)c12 ; CC(=O)OCCN(C)C(=O)Oc1cccc2[nH]cc(CCN(C)C)c12 ; COCCOCCOCCOCCC(=O)Oc1cccc2[nH]cc(CCN(C)C)c12 ; CC(=O)OCC(C)N(C)C(=O)Oc1cccc2[nH]cc(CCN(C)C)c12] 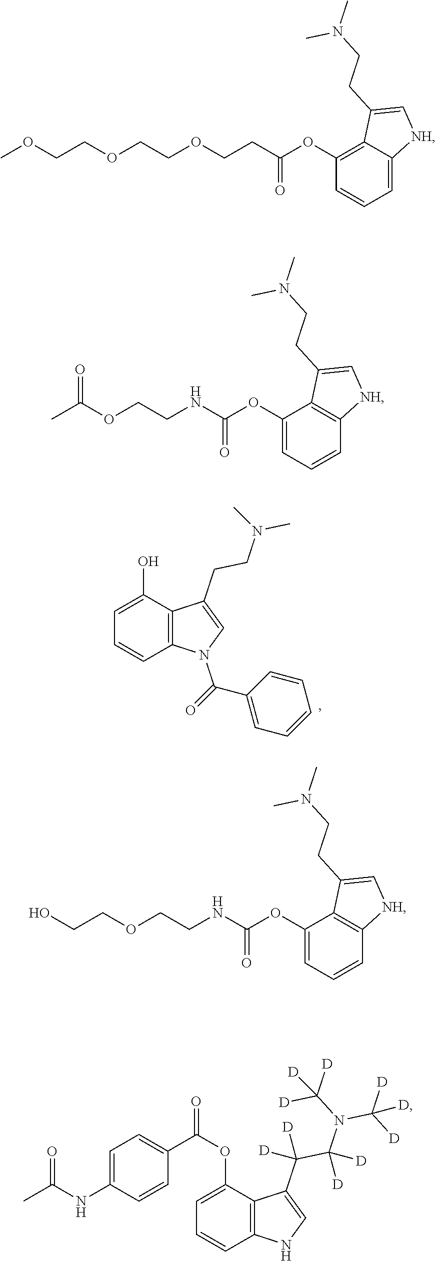 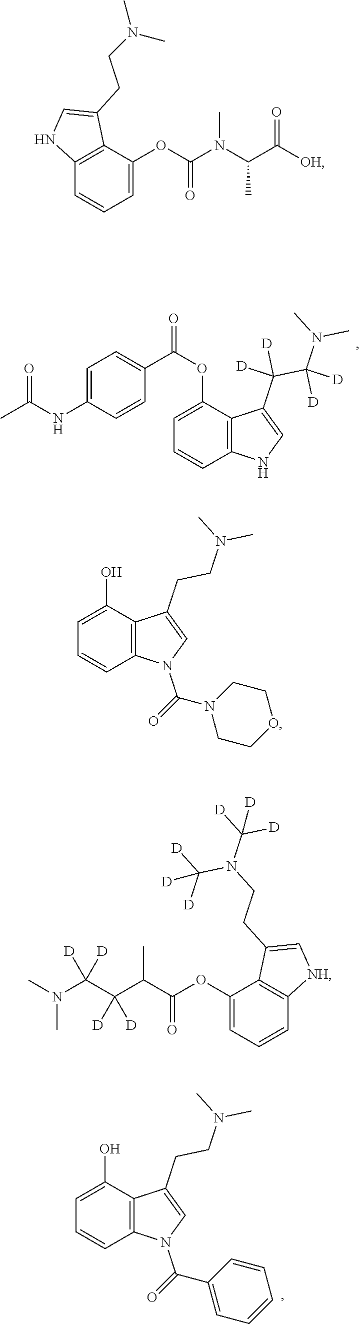 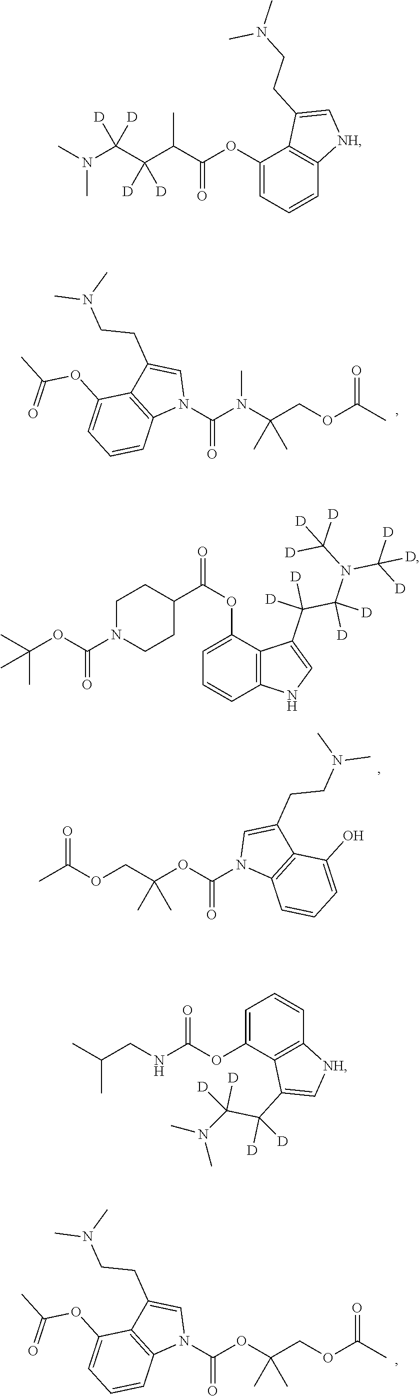 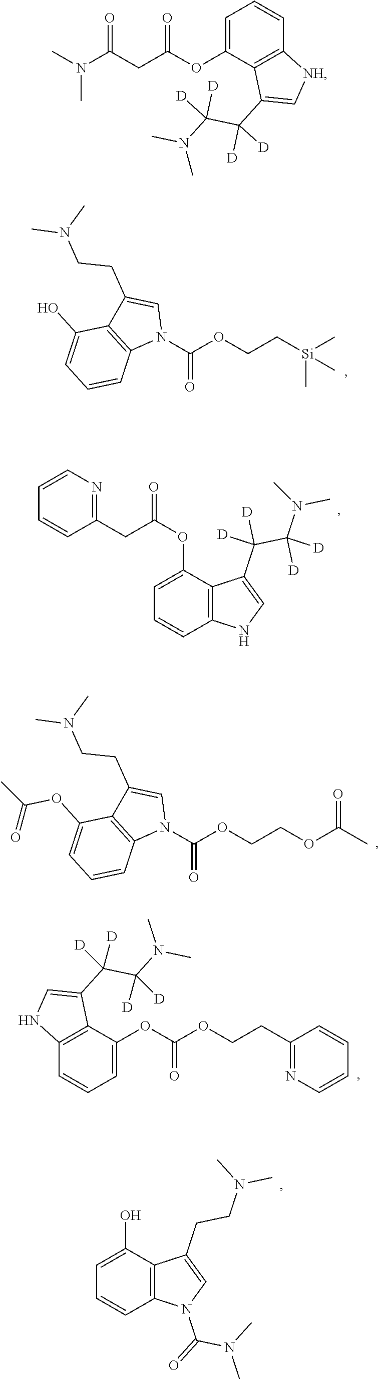 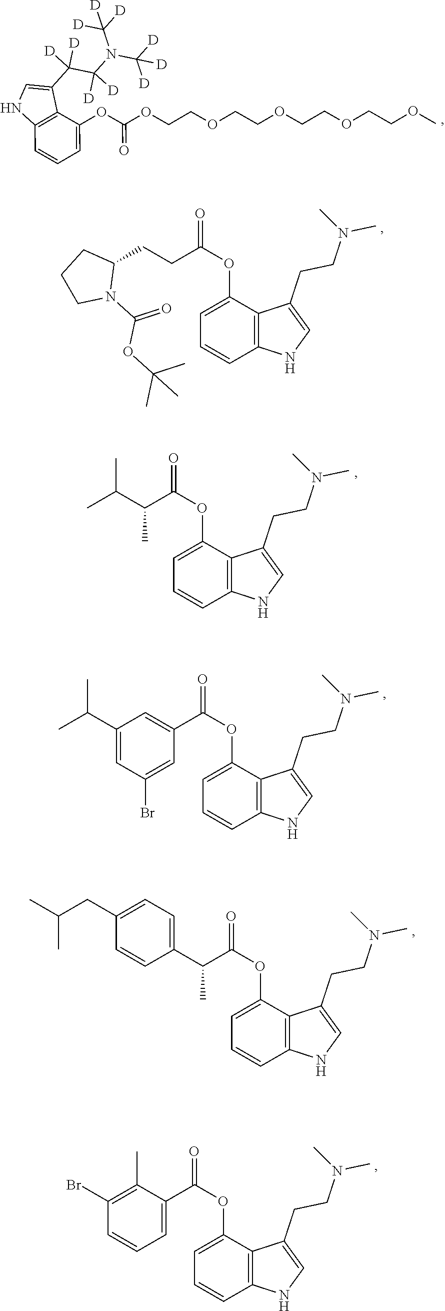 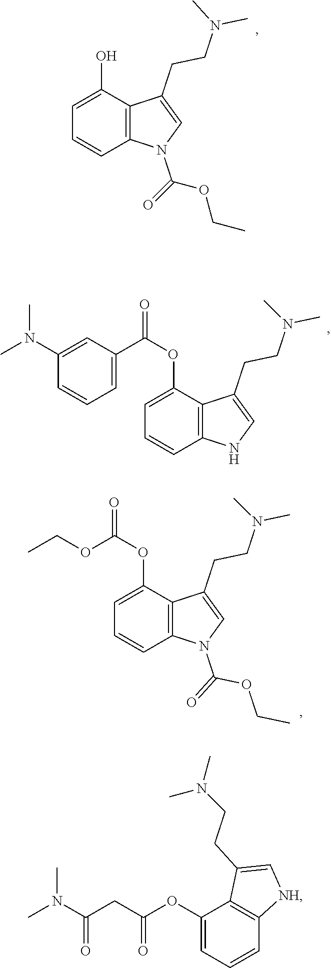 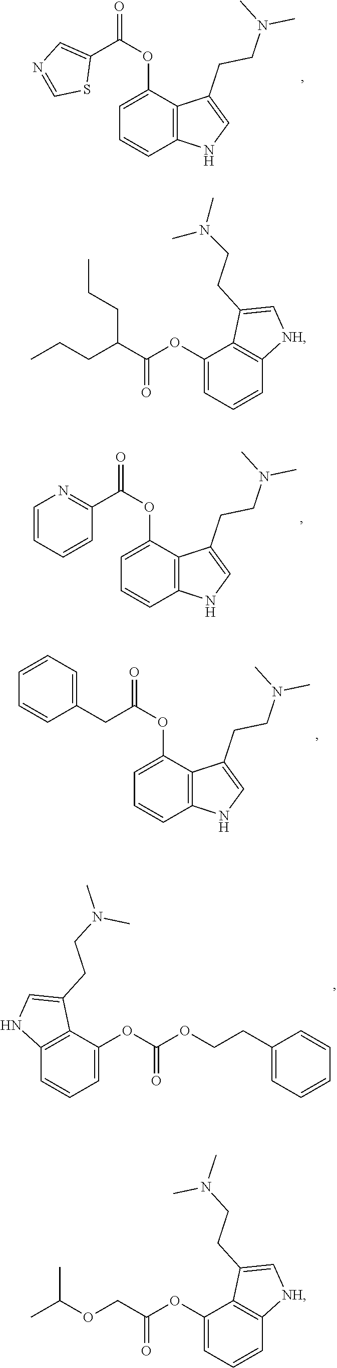 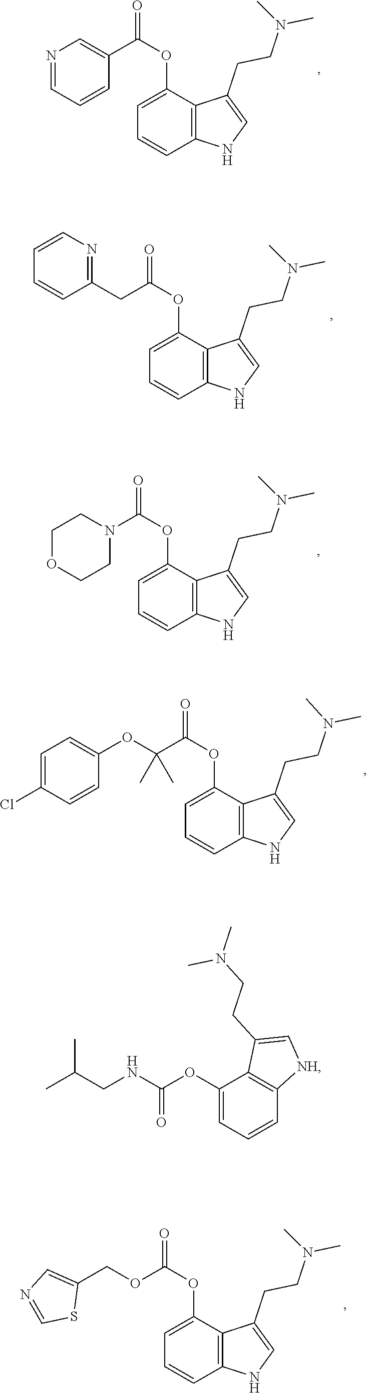 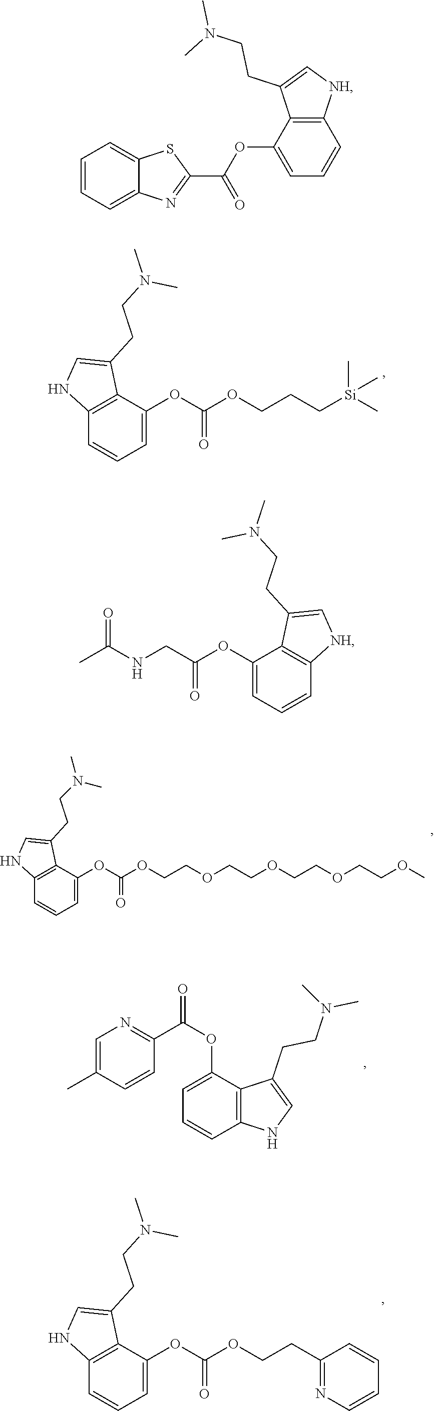 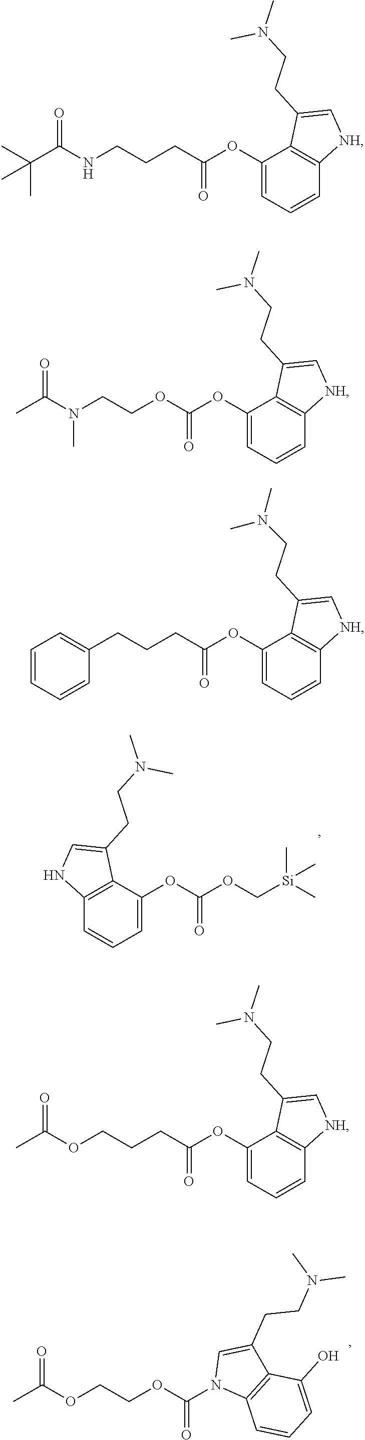 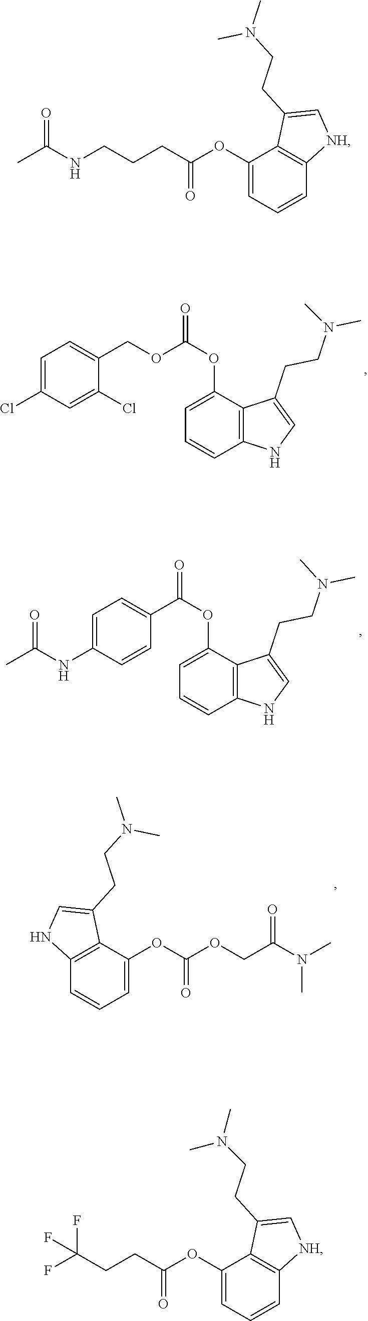 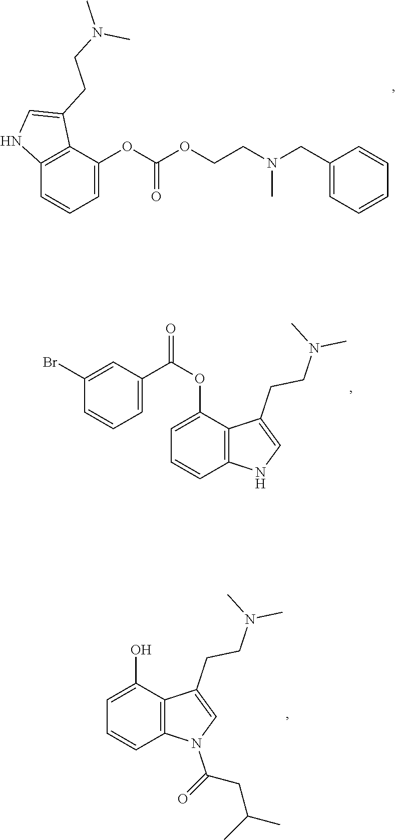 [SMILES: CN(C)CCc1c[nH]c2cccc(OC(=O)OCCN(C)Cc3ccccc3)c12 ; CN(C)CCc1c[nH]c2cccc(OC(=O)c3cccc(Br)c3)c12 ; CC(C)CC(=O)n1cc(CCN(C)C)c2c(O)cccc21]
a pharmaceutically acceptable salt thereof, or a deuterated form thereof, wherein the disease is selected from anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, anxiety disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), and premenstrual syndrome (PMS).

30-59. (canceled)
